(19) United States
(12) Patent Application Publication
(10) Pub. No.: US 2024/0077499 A1
(43) Pub. Date: Mar. 7, 2024

(54) METHODS FOR CARDIOVASCULAR DISEASE IN RHEUMATOID ARTHRITIS

(71) Applicants: Laboratory Corporation of America Holdings, Burlington, NC (US); UAB Research Foundation, Birmingham, AL (US)

(72) Inventors: JEFFREY R. CURTIS, Birmingham, AL (US); ALEXANDER GUTIN, Salt Lake City, UT (US); JERRY LANCHBURY, Salt Lake City, UT (US); ERIC SASSO, Salt Lake City, UT (US); DARL FLAKE, Salt Lake City, UT (US); ELENA HITRAYA, Salt Lake City, UT (US); CHERYL CHIN, South San Francisco, CA (US)

(21) Appl. No.: 17/471,513

(22) Filed: Sep. 10, 2021

Related U.S. Application Data
(63) Continuation of application No. PCT/US20/22294, filed on Mar. 12, 2020.
(60) Provisional application No. 62/818,017, filed on Mar. 13, 2019.
(60) Provisional application No. 62/929,459, filed on Nov. 1, 2019.

(51) Int. Cl. G01N 33/68; G16H 50/30

(52) U.S. Cl. CPC G01N 33/6893; G16H 50/30; G01N 2333/575; G01N 2333/70578; G01N 2333/96494; G01N 2800/32; G01N 2800/60

(57) ABSTRACT

This invention includes methods for assessing and treating risk of cardiovascular disease (CVD) in a subject with an inflammatory disease, for example rheumatoid arthritis (RA). Provided are methods for assessing risk, for recommending therapy, for prognosis and monitoring, and for treatment, which are advantageously accurate for CVD in RA. The methods include measuring quantitative data for biomarkers, calculating a CVD risk score for a subject using training data, and validating the CVD risk score with a set of validation clinical data.

### TECHNICAL FIELD

This invention relates to the fields of medicine and bioinformatics. More particularly, this invention relates to methods for assessing and treating inflammatory and autoimmune diseases, as well as associated cardiovascular disease. Methods for assessing risk of cardiovascular disease in patients with an inflammatory disease such as rheumatoid arthritis are disclosed.

### BACKGROUND

Rheumatoid arthritis (RA) is an inflammatory disease and a chronic, systemic autoimmune disorder affecting millions of people worldwide. In RA, the immune system targets the subject's joints as well as other organs including lung, blood vessels, and pericardium, leading to inflammation of the joints, which is arthritis, widespread endothelial inflammation, and destruction of joint tissue.

There is an association between RA and cardiovascular disease (CVD), including myocardial infarctions (MI), heart failure, and stroke. It is estimated that almost one half of RA related deaths are a result of CVD and its underlying atherosclerosis, which itself is an inflammatory disorder. The pathogenic features common to both RA and atherosclerosis include pro-inflammatory cytokines, elevated levels of acute phase reactants, neo-angiogenesis, T-cell activation, and leukocyte adhesion molecules, as well as endothelia cell injury.

Compared to the general population, patients with RA are at increased risk for CVD. A drawback of conventional methods for assessing CVD risk in RA is that those methods often explain only a fraction of the increased CVD risk in RA. Some conventional methods do not take into account the influence of RA and its features, including inflammation, and therefore underestimate CVD risk in RA patients.

Further, few conventional methods for CVD risk prediction include RA as an independent risk factor. Some conventional methods, such as the Renolyds Risk Score, quantify systemic inflammation and include C reactive protein (CRP) as a component, however, the generalizability, accuracy, and utility of this biomarker for CVD risk prediction in RA patients is unclear.

Another drawback of conventional methods, such as the ACC/AHA pooled cohort risk equation, for the general population without RA is that few are available to clinicians in real time at the point of care where they may be most useful. This method has been shown to have flawed accuracy in a number of population-based cohorts.

Thus, risk assessment and stratification using CVD risk prediction are useful to characterize patient and general population risks and the need for intervention and treatment.

What is needed is an efficient and accurate method for predicting CVD event risk in RA patients that reflects systemic inflammation and other factors. An advantageously accessible risk stratification method can facilitate care for RA patients.

There is an urgent need for methods for assessing systemic inflammation through use of biomarkers and other factors that reflect RA disease activity and complexity, including associated CVD risk. There is a need for methods that can be efficiently brought to the point of care for RA.

### BRIEF SUMMARY

This invention provides methods for assessing cardiovascular disease (CVD) risk. In some aspects, this invention utilizes biomarkers associated with inflammatory disease that can be used in methods for assessing and treating inflammatory and autoimmune diseases, as well as associated cardiovascular disease.

In some embodiments, this invention provides methods for assessing risk of cardiovascular disease in patients with an inflammatory disease. As inflammatory disease can have associated risk of a cardiovascular disease. Methods of this invention can be used for assessing and treating cardiovascular disease associated with rheumatoid arthritis.

Embodiments of this invention can provide efficient and accurate methods for predicting CVD event risk in RA patients. The methods of this disclosure can reflect the impact of systemic inflammation in RA, as well as and other patient related factors. This invention can provide surprisingly accurate methods for predicting CVD risk in an RA patient.

Further embodiments of this disclosure provide methods for assessing systemic inflammation through use of biomarkers and other factors that reflect RA disease activity and complexity, including associated CVD risk.

Methods disclosed herein can be efficiently brought to the point of care for RA, and can expand the population of patients encompassed for treatments. In some embodiments, this invention includes surprisingly improved accuracy for assessing CVD risk in RA, which can allow patients to be reclassified as having higher risk than expected. Such patients may receive treatment who would otherwise not have been treated.

Embodiments of this invention contemplate assessing risk of CVD in a subject with an inflammatory disease by determining a CVD 3-year risk score for the subject. The subject can be a patient 40 years of age or greater having no prior history of heart attack or stroke. A CVD 3-year risk score can be determined by obtaining a VECTRA-CVD score, where the VECTRA-CVD score can be related to disease activity of RA in a subject algorithmically combined with various clinical parameters of the subject. The disease activity of RA in a subject can be determined using an Adjusted VECTRA score which combines values obtained for various biomarkers algorithmically combined with various clinical parameters of the subject.

A CVD 3-year risk score can be used for assessing risk of CVD in a subject with an inflammatory disease such as RA.

A CVD 3-year risk score can be used to provide efficient and surprisingly accurate prediction and prognosis of CVD event risk in an RA patient. The CVD 3-year risk score of this disclosure can reflect the impact of systemic inflammation in RA.

A VECTRA-CVD score can be determined based on an Adjusted VECTRA score which can be algorithmically combined with clinical parameters of the subject, as well as additional adjustment terms for each of leptin, TNFRI, and MMP3 biomarkers. The pertinent clinical parameters of the subject can be age, smoking status, presence of diabetes, presence of hypertension, and history of CVD.

An Adjusted VECTRA score can be determined based on an MBDA score, which utilizes a number of VECTRA biomarkers, along with values based on clinical variables for age and sex of a subject, as well as another adjustment term for leptin biomarker.

In some aspects, VECTRA markers can be selected from chitinase 3-like 1 (cartilage glycoprotein-39) (CHI3L1); C-reactive protein, pentraxin-related (CRP); epidermal growth factor (beta-urogastrone) (EGF); interleukin 6 (interferon, beta 2) (IL6); leptin (LEP); matrix metallopeptidase 1 (interstitial collagenase) (MMP1); matrix metallopeptidase 3 (stromelysin 1, progelatinase) (MMP3); resistin (RETN); serum amyloid A1 (SAA1); tumor necrosis factor receptor superfamily, member 1A (TNFRSF1A); vascular cell adhesion molecule 1 (VCAM1); and, vascular endothelial growth factor A (VEGFA).

In further aspects, this disclosure describes methods for assessing risk of CVD in a subject having an inflammatory disease, which may be RA. The methods include assaying a sample obtained from the subject to detect the levels of at least two VECTRA markers, and calculating a CVD risk score for the subject from the biomarker levels and clinical values using an interpretation function. The CVD risk score can be CVD 3-year risk score.

In additional aspects, this disclosure describes methods for recommending therapy for a subject having an inflammatory disease, which may be RA, and may be at risk of cardiovascular disease (CVD). The methods include assaying a sample obtained from the subject to detect the levels of at least two VECTRA markers, and calculating a CVD risk score for the subject from the biomarker levels and clinical values using an interpretation function. The method can include recommending a therapy for CVD based on the CVD risk score exceeding a threshold level, or recommending no therapy for CVD based on the CVD risk score being below a threshold level. The CVD risk score can be CVD 3-year risk score.

In additional aspects, this disclosure describes methods for identifying a subject having an inflammatory disease and at risk of cardiovascular disease (CVD) who benefits from a treatment. The methods include assaying a sample obtained from the subject to detect the levels of at least two VECTRA markers, and calculating a CVD risk score for the subject from the biomarker levels and clinical values using an interpretation function. The method can include identifying the subject having an inflammatory disease and at risk of cardiovascular disease (CVD) who benefits from a treatment for CVD based on the CVD risk score exceeding a threshold level. The CVD risk score can be CVD 3-year risk score.

In further aspects, this disclosure describes methods for treating cardiovascular disease (CVD) in a subject having an inflammatory disease and in need thereof. The methods include assaying a sample obtained from the subject to detect the levels of at least two VECTRA markers, and calculating a CVD risk score for the subject from the biomarker levels and clinical values using an interpretation function. The method can include identifying the subject having an inflammatory disease and at risk of cardiovascular disease (CVD) based on the CVD risk score exceeding a threshold level and administering to the subject a treatment for CVD and/or RA. The method can include a step of monitoring the subject for the signs and symptoms of CVD for a period of time before administering to the subject a treatment for CVD and/or RA. The CVD risk score can be CVD 3-year risk score.

In additional aspects, this disclosure describes methods for monitoring a response of a subject having an inflammatory disease and at risk of having CVD to a treatment. The methods include assaying a sample obtained from the subject to detect the levels of at least two VECTRA markers, and calculating a CVD risk score for the subject from the biomarker levels and clinical values using an interpretation function. The method can include identifying the subject having an inflammatory disease and at risk of cardiovascular disease (CVD) based on the CVD risk score exceeding a threshold level. The CVD risk score can be CVD 3-year risk score.

In further aspects, this disclosure describes methods for prognosing a subject having an inflammatory disease and at risk of having CVD. The methods include assaying a sample obtained from the subject to detect the levels of at least two VECTRA markers, and calculating a CVD risk score for the subject from the biomarker levels and clinical values using an interpretation function. The method can include prognosing the subject as having a poor prognosis for CVD based on the CVD risk score exceeding a threshold level. The CVD risk score can be CVD 3-year risk score.

Embodiments of this invention include kits for assessing risk of cardiovascular disease (CVD) in a subject having an inflammatory disease, comprising reagents for measuring in a sample from the subject levels for two or more VECTRA biomarkers, and instructions for using the reagents for obtaining the biomarker levels.

This invention encompasses a system for assessing risk of cardiovascular disease (CVD) in a subject having an inflammatory disease comprising one or more processors for carrying out the steps of calculating a CVD risk score for the subject from the biomarker protein levels and one or more clinical terms using an interpretation function, and identifying the subject having an inflammatory disease and at risk of cardiovascular disease (CVD) based on the CVD risk score exceeding a threshold level, along with a display.

This invention further encompasses a non-transitory machine-readable storage medium having stored therein instructions for execution by a processor which cause the processor to perform the steps of a method for assessing risk of cardiovascular disease (CVD) in a subject having an inflammatory disease, the method comprising receiving measured protein levels from a sample from the subject for two or more VECTRA biomarkers, calculating a CVD risk score for the subject from the biomarker protein levels and one or more clinical terms using an interpretation function, identifying the subject having an inflammatory disease and at risk of cardiovascular disease (CVD) based on the CVD risk score exceeding a threshold level, and sending to a processor output for displaying and/or reporting the CVD risk score.

In some embodiments of each of the above aspects, the at least two markers may comprise two or more of CHI3L1; CRP; EGF; IL6; LEP; MMP1; MMP3; RETN; SAA1; TNFR1; VCAM1; and, VEGFA.

In some embodiments, the test score may be compared to a clinical assessment. In some embodiments, the clinical assessment can be selected from the group consisting of: a DAS, a DAS28, a DAS28-CRP, a DAS28-ESR, a Sharp score, a tender joint count (TJC), and a swollen joint count (SJC). In some embodiments, assaying may comprise performing a multiplex assay.

In some embodiments, assaying may comprise obtaining the sample, wherein the sample comprises the protein markers; contacting the sample with a plurality of distinct reagents; generating a plurality of distinct complexes between the reagents and markers; and detecting the complexes to detect the levels.

A sample in the methods of this invention can be a blood sample.

In additional embodiments, at least one clinical variable can be selected from age, gender, sex, smoking status, adiposity, body mass index (BMI), serum leptin, and race/ethnicity. A clinical variable may be one or more of age, sex, and race. A clinical variable can be one or more of age and sex.

In some embodiments, a first therapy regimen may be selected from a therapy regimen being administered to the subject at the time the sample was obtained; cessation of a therapy regimen being administered to the subject at the time the sample was obtained; tapering of a therapy regimen being administered to the subject at the time the sample was obtained; or no therapy regimen. In further embodiments, a second therapy regimen can be selected from statin drugs, modified diet, and EKG.

Embodiments of this invention include the following:

A method for assessing risk of cardiovascular disease (CVD) in a subject having an inflammatory disease, the method comprising: measuring in a sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); calculating a CVD risk score for the subject with an interpretation function using the protein levels, one or more clinical terms, and a set of training clinical data of a reference group, wherein the three or more biomarkers comprise LEP, TNFR1, and MMP3; and validating the CVD risk score with an interpretation function using the protein levels, one or more clinical terms, and a set of validation clinical data of the reference group.

The sample may be a blood sample. The subject can be more than 40 years of age. The subject may have no prior history of heart attack or stroke. The inflammatory disease can be rheumatoid arthritis (RA). The clinical terms may comprise age, sex, smoking, diabetes, hypertension, and history of cardiovascular disease, or age, sex, smoking, diabetes, hypertension, history of cardiovascular disease, gender, adiposity, body mass index, race, and ethnicity.

The reference group can be patients who have been tested for activity of rheumatoid arthritis (RA) and/or cardiovascular disease (CVD). The reference group may be Medicare patients.

The three or more biomarkers may comprise LEP, TNFR1, MMP3, CRP, IL6, and SAA1. The three or more biomarkers can comprise LEP, TNFR1, MMP3, CRP, IL6, SAA1, CHI3L1, EGF, VCAM1, MMP1, RETN, and VEGFA.

The CVD risk score can be validated with clinical data selected from a DAS score, a DAS28 score, a DAS28-CRP score, a DAS28-ESR score, a Sharp score, a tender joint count score (TJC), and a swollen joint count score (SJC).

The calculating of a CVD risk score may comprise calculating an Adjusted MBDA score and calculating the CVD risk score by combining the Adjusted MBDA score with the clinical terms using the interpretation function.

The interpretation function may comprise one or more of Survival Regression analysis, Cox Proportional Hazards, Box-Cox transformation, Clustering Machine Learning, Hierarchical Clustering Analysis, Centroid Clustering, Distribution Clustering, Density Clustering, Cluster Data Mining, analysis of variants (ANOVA), Ada-boosting, Classification and Regression Trees (CART), boosted CART, Random Forest (RF), Recursive Partitioning Trees (RPART), Curds and Whey (CW), Curds and Whey-Lasso, principal component analysis (PCA), factor rotation analysis, Linear Discriminant Analysis (LDA), Eigengene Linear Discriminant Analysis (ELDA), quadratic discriminant analysis, Discriminant Function Analysis (DFA), Hidden Markov Models, kernel density estimation, kernel partial least squares algorithm, kernel matching pursuit algorithm, kernel Fisher's discriminate analysis algorithm, kernel principal components analysis algorithm; linear regression, Stepwise Regression, Forward-Backward Variable Stepwise Regression, Lasso shrinkage and selection, Elastic Net regularization and selection, Lasso and Elastic Net-regularized generalized linear model, Logistic Regression (LogReg), Kth-nearest neighbor (KNN), non-linear regression, classification, neural networks, partial least square, rules based classification, shrunken centroids (SC), sliced inverse regression, Standard for the Exchange of Product model data, Application Interpreted Constructs (StepAIC), super principal component (SPC) regression, Support Vector Machines (SVM), and Recursive Support Vector Machines (RSVM), and combinations thereof. The interpretation function can provide an algorithm which includes a hyperbolic tangent or an exponential of a biomarker score.

The method may include recommending a therapy for CVD for the subject based on the CVD risk score exceeding a threshold level, or recommending no therapy for CVD based on the CVD risk score being below a threshold level. The therapy can be one of: a therapy being administered to the subject at the time the sample was obtained; a cessation of therapy being administered to the subject at the time the sample was obtained; a tapering of the therapy being administered to the subject at the time the sample was obtained. The therapy can be one or more of administering a medication, administering a surgery, administering a rehabilitation, administering a treatment for a different disease or condition of the subject, and ameliorating a symptom of the subject. The therapy can be administering one or more medications selected from a cholesterol-reducing medication, a blood flow-increasing medication, a heart rhythm-regulating medication, a heart rhythm-stabilizing medication, a blood blockage-reducing medication, a beta-blocker, an ACE inhibitor, an aldosterone inhibitor, an angiotensin II receptor blocker, a calcium channel blocker, a cholesterol lowering drug, a diuretic, an inotropic medication, an electrolyte supplement, a PCSK9 inhibitor, and a vasodilator. The therapy may be administering a DMARD selected from MTX, azathioprine (AZA), bucillamine (BUC), chloroquine (CQ), cyclosporine, doxycycline (DOXY), hydroxychloroquine (HCQ), intramuscular gold (IM gold), leflunomide (LEF), levofloxacin (LEV), sulfasalazine (SSZ), folinic acid, D-pencillamine, gold auranofin, gold aurothioglucose, gold thiomalate, cyclophosphamide, chlorambucil, infliximab, adalimumab, etanercept, golimumab, anakinra, abatacept, rituximab, and tocilizumab. The therapy may be administering one or more of percutaneous coronary intervention, coronary artery bypass surgery, heart valve repair or replacement surgery, bariatric surgery, cardiac rehabilitation, therapeutic physical programs, dietary modification or restriction, smoking cessation, diabetes treatment, hypertension treatment, symptom relief, reducing risk of recurrence, reducing severity of recurrence, reducing heart failure or heart attack, and reducing stroke.

The method can include identifying of a subject having an inflammatory disease and at risk of cardiovascular disease (CVD) who benefits from a treatment for CVD based on the CVD risk score exceeding a threshold level. The threshold level can be one of borderline risk threshold, intermediate risk threshold, and high risk threshold based on a CVD 3-year risk or a CVD 10-year risk.

Embodiments of this invention further contemplate methods for treating cardiovascular disease (CVD) in a subject having an inflammatory disease and in need thereof, the method comprising: measuring in a sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); and calculating a CVD risk score for the subject with an interpretation function using the protein levels, one or more clinical terms, and a set of training clinical data of a reference group, wherein the three or more biomarkers comprise LEP, TNFR1, and MMP3; validating the CVD risk score with an interpretation function using the protein levels, one or more clinical terms, and a set of validation clinical data of the reference group; identifying the subject having an inflammatory disease and at risk of cardiovascular disease (CVD) based on the CVD risk score exceeding a threshold level; and administering to the subject a treatment for CVD or RA.

The method can include identifying the subject as having an inflammatory disease and at risk of cardiovascular disease (CVD) who benefits from a treatment for CVD based on the CVD risk score exceeding a threshold level.

In further aspects, this invention includes methods for prognosing a subject having an inflammatory disease and at risk of having CVD, the method comprising: measuring in a blood sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); and calculating a CVD risk score for the subject from the biomarker protein levels and one or more clinical terms using an interpretation function, wherein the three or more biomarkers comprise LEP, TNFR1, and MMP3; and prognosing the subject as having a poor prognosis for CVD based on the CVD risk score exceeding a threshold level.

Additional aspects include kits for assessing risk of cardiovascular disease (CVD) in a subject having an inflammatory disease, the kit comprising: reagents for measuring in a blood sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); and instructions for using the reagents for obtaining the biomarker levels.

Further aspects include systems for assessing risk of cardiovascular disease (CVD) in a subject having an inflammatory disease, the system comprising: a processor for receiving the subject's protein levels measured in a blood sample for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); one or more processors for carrying out the steps: calculating a CVD risk score for the subject from the biomarker protein levels and one or more clinical terms using an interpretation function, wherein the three or more biomarkers comprise LEP, TNFR1, and MMP3; identifying the subject having an inflammatory disease and at risk of cardiovascular disease (CVD) based on the CVD risk score exceeding a threshold level; and a display for displaying and/or reporting the CVD risk score.

In further aspects, this invention includes a non-transitory machine-readable storage medium having stored therein instructions for execution by a processor which cause the processor to perform the steps of a method for assessing risk of cardiovascular disease (CVD) in a subject having an inflammatory disease, the method comprising: receiving measured protein levels from a blood sample from the subject for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); calculating a CVD risk score for the subject from the biomarker protein levels and one or more clinical terms using an interpretation function, wherein the three or more biomarkers comprise LEP, TNFR1, and MMP3; identifying the subject having an inflammatory disease and at risk of cardiovascular disease (CVD) based on the CVD risk score exceeding a threshold level; and sending to a processor output for displaying and/or reporting the CVD risk score.

### BRIEF DESCRIPTION OF THE DRAWINGS

FIG. 1 shows a relationship between predictive VECTRA CVD score (x axis) and CVD 3-year Risk % (y axis) in an RA patient.

FIG. 2 shows a distribution of patients for the relationship in FIG. 1. The distribution in FIG. 2 shows that for every level of the predictive VECTRA CVD score, patients with a wide range of CVD 3-year Risk % were found. This distribution and relationship shows that the scores are predictively valid and meaningful for a patients over a wide range of risk levels.

FIG. 3 shows a correlation between predictive CVD risk scoring (x axis) and CVD risk patient outcome (y axis). The goodness of fit of the data points to the dashed line indicates a very strong correlation exists between scoring and patient outcome.

FIG. 4 shows a distribution of patient CVD 3-year Risk % against scores.

FIG. 5 shows results of a method for clinically validating the predictive ability of CVD 3-year Risk % values of this invention. FIG. 5 shows CVD-event-free survival rates for RA subjects. The plotted lines show survival rates corresponding to low, intermediate, and high CVD risk group thresholds for the predictive CVD 3-year Risk % values of this invention (n=10,275).

FIG. 6 shows results of a method for clinically validating the predictive ability of CVD 3-year Risk % values of this invention. FIG. 6 shows CVD-event-free survival rates for RA subjects. The plotted lines show survival rates corresponding to low/borderline, intermediate, and high CVD risk group thresholds for the predictive CVD 3-year Risk % values of this invention (n=10,275).

FIG. 7 shows results of a method for clinically validating the predictive ability of CVD 3-year Risk % values of this invention. FIG. 7 shows CVD-event-free survival rates for RA subjects. The plotted lines show survival rates corresponding to low, borderline, intermediate, and high CVD risk group thresholds for the predictive CVD 3-year Risk % values of this invention (n=10,275).

FIG. 8 shows the superior accuracy of a method of this invention for assessing CVD 3-year Risk % values for RA subjects. The bar chart in FIG. 8 shows that the method of this invention based on VECTRA CVD Score (“MBDA-based”) was surprisingly more accurate than various methods that did not include VECTRA CVD Score. The bar on the left shows that a determination of CVD risk based only on the clinical parameters age and sex was far less accurate than using a VECTRA CVD Score. This may be seen by the incremental increases in the likelihood ratio test (LRT) (y axis, height of shaded portion) from left to right in FIG. 8. Likewise, the second, third and fourth bars from the left also show that determination of CVD risk based only on the parameters age+sex+CRP(biomarker), a set of clinical parameters without CRP, and a set of clinical parameters with CRP, respectively, were far less accurate than using a VECTRA CVD Score. The set of clinical parameters was age, sex, diabetes, hypertension, smoking, and history of CVD. In sum, FIG. 8 shows by bivariate analysis, that the use of the VECTRA CVD Score of this invention may be necessary to achieve very high levels of accuracy in determining CVD 3-year Risk % values for RA subjects.

FIG. 9 shows the superior accuracy of a method of this invention for assessing CVD 3-year Risk % values for RA subjects. The bar chart in FIG. 9 shows that the method of this invention based on VECTRA CVD Score (“MBDA-based”) was surprisingly more accurate than various methods that did not include VECTRA CVD Score. The bar on the left shows that a determination of CVD risk based only on the parameters age+sex+CRP(biomarker) was far less accurate than using a VECTRA CVD Score. This may be seen by the incremental increases in the likelihood ratio test (LRT) (y axis, height of shaded portion) from left to right in FIG. 9. Likewise, the second and third bars from the left also show that determination of CVD risk based only on a set of clinical parameters without CRP, and a set of clinical parameters with CRP, respectively, were far less accurate than using a VECTRA CVD Score. The set of clinical parameters was age, sex, diabetes, hypertension, smoking, and history of CVD. In sum, FIG. 9 shows by bivariate analysis, that the use of the VECTRA CVD Score of this invention may be necessary to achieve very high levels of accuracy in determining CVD 3-year Risk % values for RA subjects.

FIG. 10 shows a receiver-operator curve for the model described in Tables 28-30 at 183 days.

FIG. 11 shows a receiver-operator curve for the model described in Tables 28-30 at 365 days.

FIG. 12 shows a receiver-operator curve for the model described in Tables 28-30 at 548 days.

FIG. 13 shows a receiver-operator curve for the model described in Tables 28-30 at 730 days.

FIG. 14 shows a receiver-operator curve for the model described in Tables 28-30 at 1095 days.

FIG. 15 shows a receiver-operator curve for the model described in Tables 31-33 at 183 days.

FIG. 16 shows a receiver-operator curve for the model described in Tables 31-33 at 365 days.

FIG. 17 shows a receiver-operator curve for the model described in Tables 31-33 at 548 days.

FIG. 18 shows a receiver-operator curve for the model described in Tables 31-33 at 730 days.

FIG. 19 shows a receiver-operator curve for the model described in Tables 31-33 at 1095 days.

FIG. 20 shows biomarkers used to predict each DAS28-CRP component.

The skilled artisan will understand that the drawings described above are for purposes of illustration and are not intended to limit the scope of the disclosure.

### DETAILED DESCRIPTION OF THE DISCLOSURE

This invention includes methods for assessing and treating risk of cardiovascular disease (CVD) in a subject with an inflammatory disease, for example rheumatoid arthritis (RA). Disclosed are risk analysis methods for prognosis and treatment of CVD and/or RA, which are advantageously accurate for CVD in RA.

The methods of this invention include performing assays to generate risk analysis based on quantitative data for biomarkers of inflammatory disease activity and additional clinical variables of a subject to further assess and treat CVD risk and/or RA.

Embodiments of this invention further provide bioinformatic methods for determining risk of cardiovascular disease or myocardial infarction. The risk of cardiovascular disease can be determined in the general population, as well as for patients with an inflammatory disease such as rheumatoid arthritis.

The methods of this disclosure can provide a CVD risk score based on biomarkers of inflammatory disease activity, as well as various clinical variables pertinent to a subject. The methods can take into account the influence of RA and its features, including inflammation. In such embodiments, accounting for the effects of inflammation provides surprisingly accurate measures and prognosis of CVD risk in RA patients.

This disclosure provides various methods for assessing and treating risk of CVD in RA. The methods can generate and utilize an adjusted, multi-biomarker disease activity score, for example, an Adjusted MBDA score, in combination with certain clinical variables.

As used herein, the term “Adjusted MBDA score” can refer to an Adjusted VECTRA score.

In some aspects, this invention can utilize various biomarkers for determining risk of cardiovascular disease.

In further aspects, this invention can utilize various biomarkers that can be associated with subjects having an inflammatory and/or autoimmune disease, for example RA.

In certain aspects, this invention can utilize various biomarkers for determining or assessing cardiovascular disease (CVD) risk in a subject having an inflammatory and/or autoimmune disease, for example RA.

In additional aspects, this invention can utilize various biomarkers for determining CVD risk of RA patients upon their response to an inflammatory disease therapy and/or CVD therapy.

In some embodiments, this disclosure can provide an efficient and accurate algorithm for predicting CVD event risk in an RA patient. The algorithm for predicting CVD event risk can take into account effects of systemic inflammation.

In further embodiments, this invention includes an accessible risk stratification algorithm that can facilitate care for RA patients. This disclosure includes methods that can be efficiently brought to the point of care for CVD and/or RA.

In additional embodiments, this disclosure describes methods to assess systemic inflammation through the use of biomarkers that reflect RA disease activity.

In certain embodiments of this invention, an adjusted, multi-biomarker disease activity score, such as an adjusted MBDA score, can be used to predict likelihood or risk of CVD in RA.

In some aspects, this disclosure shows that in RA the risk for CVD events may be reduced in patients with lower RA disease activity. Embodiments of this invention therefore contemplate RA patient management by intervening directly upon inflammatory pathways to reduce CVD risk.

In further aspects, this invention can show benefits to RA patients in methods for reducing systemic inflammation via immunologic means. In some embodiments, patients having elevated C reactive protein (CRP) can be treated by directly lowering systemic inflammation to reduce CVD event risk.

In additional aspects, this invention can provide methods for CVD risk stratification in RA that can be used to identify high risk patients for medical treatment. CVD risk stratification can encompass threshold values determined by validation of CVD risk scores with clinical outcomes.

In certain aspects of this invention, methods for determining a CVD risk may be used by clinicians in real time and/or at the point of care.

In further aspects, a method of this invention for assessing CVD risk can be used to take into account the influence of RA and its features, including inflammation. Methods for CVD risk can accurately estimate a CVD risk in an RA patient by including RA as an independent risk factor.

In additional aspects, methods of this invention for assessing and utilizing CVD risk prediction may quantify systemic inflammation and include CRP as an analytic component. In some embodiments, methods for assessing and utilizing CVD risk prediction can provide surprisingly accurate CVD event risk values in RA patients by including effects of systemic inflammation. Such methods may assist in making risk stratification more accessible and facilitate care for RA patients.

In further embodiments, systemic inflammation may be included in methods for assessing CVD risk in RA by using one or more biomarkers that reflect RA disease activity. In certain embodiments, an adjusted, multi-biomarker disease activity score, such as an adjusted MBDA score, can be used to provide results that are highly correlated with RA disease activity, as measured by the Disease Activity Score in 28 joints (DAS28), among other scores.

Embodiments of this invention further contemplate methods and algorithms for CVD risk assessment that may be useful to clinicians. More particularly, this invention includes methods for measurement of disease markers and training and validation of algorithms using markers and clinical variables to assess RA disease activity.

Methods and algorithms of this disclosure for assessing CVD risk may include determining and validating a CVD risk score using various clinical data that would be accessible to clinicians or available in electronic systems. For example, clinical features and data may be contained in electronic health records or health plan claims data.

Methods and algorithms of this disclosure may provide a validated CVD risk score having prognostic and/or outcome predictive utility. Such methods and algorithms for CVD risk score may combine the use of clinical features or data with RA biomarkers. A CVD risk score may be used for assessing CVD risk in an RA patient, recommending a therapy, identifying a subject who benefits from a treatment, monitoring a response of a patient to a treatment, or prognosing a patient, as well as treating a patient for inflammatory disease and/or CVD.

The methods of this invention for determining a CVD risk in an RA patient may provide surprisingly accurate CVD risk assessment.

In certain embodiments, accurate CVD risk assessment may advantageously expand the population of patients that are treated for CVD and/or RA. By expanding the population of patients that are treated for CVD and/or RA, this invention can provide treatment for patients who may not have been treated by conventional methods or modalities for CVD risk. An expansion of the population of patients that are treated for CVD and/or RA can be shown by reclassification of patients according to a CVD risk score threshold of this invention.

By encompassing systemic inflammation, which is a significant component of RA that contributes to CVD risk, the methods of this invention can expand the population of patients that can be assessed or treated for CVD and/or RA. The methods of this invention can significantly improve the practice of medicine and therapeutic modalities for CVD risk in general, and specifically for CVD risk in RA patients.

As used herein, the term “disease” refers to any disorder, condition, sickness, ailment that manifests in, for example, a disordered or incorrectly functioning organ, part, structure, or system of the body, and results from, e.g., genetic or developmental errors, infection, poisons, nutritional deficiency or imbalance, toxicity, or unfavorable environmental factors.

As used herein, the term “autoimmune disease” encompasses any disease resulting from an immune response against substances and tissues normally present in the body. Examples of suspected or known autoimmune diseases include rheumatoid arthritis, early rheumatoid arthritis, axial spondyloarthritis, juvenile idiopathic arthritis, seronegative spondyloarthropathies, ankylosing spondylitis, psoriatic arthritis, antiphospholipid antibody syndrome, autoimmune hepatitis, Behget's disease, bullous pemphigoid, coeliac disease, Crohn's disease, dermatomyositis, Goodpasture's syndrome, Graves' disease, Hashimoto's disease, idiopathic thrombocytopenic purpura, IgA nephropathy, Kawasaki disease, systemic lupus erythematosus, mixed connective tissue disease, multiple sclerosis, myasthenia gravis, polymyositis, primary biliary cirrhosis, psoriasis, scleroderma, Sjögren's syndrome, ulcerative colitis, vasculitis, Wegener's granulomatosis, temporal arteritis, Takayasu's arteritis, Henoch-Schonlein purpura, leucocytoclastic vasculitis, polyarteritis nodosa, Churg-Strauss Syndrome, and mixed cryoglobulinemic vasculitis.

As used herein, the term “inflammatory disease” refers to any disease resulting from the biological response of vascular tissues to harmful stimuli, including but not limited to such stimuli as pathogens, damaged cells, irritants, antigens and, in the case of autoimmune disease, substances and tissues normally present in the body.

Some examples of inflammatory disease include rheumatoid arthritis (RA), juvenile idiopathic arthritis, ankylosing spondylitis, psoriatic arthritis, atherosclerosis, asthma, autoimmune diseases, chronic inflammation, chronic prostatitis, glomerulonephritis, hypersensitivities, inflammatory bowel diseases, pelvic inflammatory disease, reperfusion injury, transplant rejection, and vasculitis.

As used herein, the terms “cardiovascular disease,” “cardiovascular disorder,” and “CVD” in general may classify various conditions affecting the heart, heart valves, and vasculature, for example, arteries and veins, of the body and encompasses diseases, conditions, and outcomes or events including, but not limited to arteriosclerosis, atherosclerosis, myocardial infarction (MI), acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

Diseases and medical conditions of this disclosure include rheumatoid arthritis (RA) and cardiovascular diseases (CVDs). CVDs can include atherosclerosis, coronary atherosclerosis, carotid atherosclerosis, hypertension. For example, CVDs can include pulmonary hypertension, labile hypertension, idiopathic hypertension, low-renin hypertension, salt-sensitive hypertension, low-renin hypertension, thromboembolic pulmonary hypertension, pregnancy-induced hypertension, renovascular hypertension, hypertension-dependent end-stage renal disease, hypertension associated with cardiovascular surgical procedures, and hypertension with left ventricular (LV) hypertrophy, LV diastolic dysfunction, unobstructive coronary heart diseases, myocardial infarctions, cerebral infarctions, peripheral vascular disease, cerebrovascular disease, cerebral ischemia, angina, including chronic, stable, unstable and variant (Prinzmetal) angina pectoris, aneurysm, ischemic heart disease, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiography, vascular grafting, coronary artery bypass surgery, thromboembolic events, post-angioplasty restenosis, coronary plaque inflammation, hypercholesterolemia, hypertriglyceridemia, embolism, stroke, shock, arrhythmia, atrial fibrillation or atrial flutter, thrombotic occlusion and reclusion cerebrovascular incidents, left ventricular dysfunction, cardiac hypertrophy, and hypertension with left ventricular hypertrophy and/or unobstructive CVD.

Examples of CVDs can include conditions associated with oxidative stress, microvascular coronary heart disease, coronary endothelial dysfunction, left ventricular hypertrophy, dyspnea, inflammation, diabetes, and chronic renal failure. Other CVD medical conditions are generally known to one of ordinary skill in the art.

As used herein, the term “remission” refers to the state of absence of disease activity in patients known to have a chronic illness that usually cannot be cured. The term “sustained clinical remission” or “SC-REM” as used herein refers to a state of clinical remission sustained as evaluated based on clinical assessments, for example, DAS28 for at least six months. The term “functional remission” as used herein refers to a state of remission as evaluated using functional assessment measures such as but not limited to HAQ. Sustained remission can be used interchangeably with maintained remission.

Methods for clinically diagnosing diseases and medical conditions can be known to one of skill in the art.

In general, ultrasound measurements of carotid artery intima-media thickness (IMT) can be used as a measurement of a CVD, for example, atherosclerosis, and/or as a surrogate endpoint for determining regression or progression of atherosclerotic CVD, especially carotid atherosclerosis.

In general, carotid IMT (CIMT) measures the thickness of carotid artery walls to detect the presence of atherosclerosis or atherosclerosis burden and progression of atherosclerosis, and can be a surrogate endpoint for evaluating the presence and progression of atherosclerotic CVD. Carotid IMT measurements may be obtained from one or more segments of the carotid artery: in the common carotid, at the bifurcation, or in the internal carotid artery. The IMT of the common carotid artery (CCA), in particular, may be useful as an atherosclerosis risk marker. See, e.g., E. Vicenzini et al., J. Ultrasound Med. 2007, 26:427-432. Atherosclerosis burden within the artery, as measured by carotid IMT, can be related to CVD risk, and may be shown to predict fatal coronary death. See, e.g., J T Salonen and R. Salonen, Arterioscler. Thromb. 1991, 11: 1245-1249; LE Chambless et al., Am. J. Epidemiol. 1997, 146: and, H N Hodis et al., Ann. Intern. Med. 1998, 128: 262-269. Absolute intima-media thickness related to risk for clinical coronary events. Carotid IMT measurements, therefore, can be used to determine atherosclerosis burden in a subject, and changes in IMT can also be used to evaluate changes in atherosclerosis burden, and atherosclerosis progression.

Embodiments of this invention further contemplate methods for treating CVD in RA, as well as for treating RA.

Methods for treating CVD in RA can include one or more steps for administering CVD medication, administering cholesterol-reducing medication, administering blood flow-increasing medication, administering heart rhythm-regulating medication, administering heart rhythm-stabilizing medication, and/or administering blood blockage-reducing medication.

Methods for treating CVD in RA can include a therapy regimen being administered to the subject.

Examples of a therapy regimen for CVD in RA can include a therapy regimen that was being administered at the time the sample was obtained. The therapy regimen that was being administered may have been a therapy regimen for RA, or CVD, or both.

Examples of a therapy regimen for CVD in RA can include cessation of a therapy regimen that was being administered to the subject at the time the sample was obtained. The therapy regimen that was being administered may have been a therapy regimen for RA, or CVD, or both.

Examples of a therapy regimen for CVD in RA can include tapering of a therapy regimen that was being administered to the subject at the time the sample was obtained. The therapy regimen that was being administered may have been a therapy regimen for RA, or CVD, or both.

Examples of CVD medication include beta-blockers, ACE inhibitors, aldosterone inhibitors, angiotensin II receptor blockers, calcium channel blockers, cholesterol lowering drugs, diuretics, inotropic medications, electrolyte supplements, PCSK9 inhibitors, and vasodilators.

Examples of CVD medication include metoprolol, acebutolol, atenolol, bisoprolol, and propranolol.

Examples of CVD medication include lisinopril, enalapril, fosinopril, moexipril, perindopril, quinapril, ramipril, and tranolapril.

Examples of CVD medication include spironolactone and eplerenone.

Examples of CVD medication include azilsartan, eprosartan, irbesartan, telmisartan, candesartan, losartan, Olmesartan, and valsartan.

Examples of CVD medication include amlodipine, felodipine, isradipine, nicardipine, nifedipine, diltiazem, nisoldipine, and verapamil.

Examples of CVD medication include atorvastatin, fluvastatin, lovastatin, pitavastatin, pravastatin, rosuvastatin, and simvastatin.

Examples of CVD medication include bumetanide, ethacrynic acid, furosemide, torsemide, chlorothiazide, chlorthalidone, hydrochlorothiazide, metolazone, indapamide, amiloride, and triamterene.

Examples of CVD medication include amrinone, digoxin, dobutamine, dopamine, inamrinone, inotropin, lanoxin, and milrinone.

Examples of CVD medication include alirocumab and evolocumab.

Examples of CVD medication include alprostadil, riociguat, hydralazine, minoxidil, nesiritide, and nitroprusside.

Examples of CVD medication include aspirin, clopidogrel, warfarin, potassium supplements, and calcium supplements.

Methods for treating CVD in RA can include steps for one or more of administering surgery, percutaneous coronary intervention, coronary artery bypass surgery, heart valve repair or replacement surgery, and/or administering bariatric surgery.

Methods for treating CVD in RA can include steps for one or more of administering cardiac rehabilitation, therapeutic physical programs, and/or dietary modification or restriction.

Methods for treating CVD in RA can include steps for treating risk factors of CVD including one or more of smoking cessation, diabetes treatment, and/or hypertension treatment.

Methods for treating CVD in RA can include steps for administering one or more of symptom relief, reducing risk of recurrence, reducing severity of recurrence, reducing heart failure or heart attack, and/or reducing stroke.

Methods for treating RA can include steps for administering RA medication. Examples of RA medication include administering an NSAID drug, administering a steroid drug, administering a disease-modifying antirheumatic drug DMARD, and administering a biologic DMARD.

Examples of RA medication include ibuprofen, naproxen, and meloxicam.

Examples of RA medication include prednisone.

Examples of RA medication include methotrexate, leflunomide, hydroxychloroquine, and sulfasalazine.

Examples of RA medication include abatacept, adalimumab, anakinra, baricitinib, certolizumab, etanercept, golimumab, infliximab, rituximab, sarilumab, tocilzumab, and tofacitinib.

Methods for treating RA can include steps for administering surgery, synovectomy surgery, tendon repair surgery, joint fusion surgery, joint replacement surgery, and bariatric surgery.

Methods for treating RA can include steps for administering therapeutic physical programs, and dietary modification or restriction.

Methods for treating RA can include steps for monitoring a response of a subject to a treatment.

Therapies for CVD can include, without limitation, anticoagulants, antiplatelet agents, thrombolytic agents, antithrombotics, antiarrhythmic agents, agents that prolong repolarization, antihypertensive agents, vasodilator, antihypertensives, diuretics, inotropic agents, antianginal agents and the like.

Examples of anticoagulants include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

Examples of antiplatelet agents include aspirin, a dextran, dipyridamole (persantin), heparin, sulfinpyranone (anturane), clopidrogel and ticlopidine (ticlid).

Examples of thrombolytic agents include tissue plaminogen activator (activase), plasmin, pro-urokinase, urokinase (abbokinase) streptokinase (streptase), anistreplase/APSAC (eminase).

To identify additional therapeutics or drugs that are appropriate for a specific subject, a test sample from the subject can also be exposed to a therapeutic agent or a drug, and the level of one or more biomarkers can be determined. The level of one or more biomarkers can be compared to sample derived from the subject before and after treatment or exposure to a therapeutic agent or a drug, or can be compared to samples derived from one or more subjects who have shown improvements in inflammatory disease state or activity (e.g., clinical parameters or traditional laboratory risk factors) as a result of such treatment or exposure.

The term “administering” as used herein includes, but is not limited to, the placement of a composition into a subject by a method or route that results in at least partial localization of the composition at a desired site such that a desired effect is produced. Routes of administration include both local and systemic administration. Generally, local administration results in more of the composition being delivered to a specific location as compared to the entire body of the subject, whereas, systemic administration results in delivery to essentially the entire body of the subject. “Administering” also includes performing physical actions on a subject's body, including massage, physical therapy, etc.

In further embodiments, this invention contemplates methods for assessing specific risk of cardiovascular disease in patients with an inflammatory disease. The inflammatory disease can be RA. Additional methods are disclosed for recommending specific therapy for a subject having an inflammatory disease, which may be RA, and may be at risk of cardiovascular disease (CVD). Further methods include identifying a specific subject having an inflammatory disease and at risk of cardiovascular disease (CVD) who benefits from a treatment. This disclosure includes specific methods for treating cardiovascular disease (CVD) in a subject having an inflammatory disease and in need thereof. Additional methods include monitoring a specific response of a subject having an inflammatory disease and at risk of having CVD to a treatment. Further methods encompass prognosing a subject having an inflammatory disease and at risk of having CVD.

Methods of this invention can encompass efficient and accurate methods for predicting CVD event risk in RA patients. The methods can include assessing systemic inflammation through use of biomarkers and other factors that reflect RA disease activity and complexity, including associated CVD risk. Methods disclosed herein can be efficiently brought to the point of care for RA, and can expand the population of patients encompassed for treatments.

Embodiments of this invention contemplate assessing risk of CVD in a subject with an inflammatory disease by determining a CVD risk value for the subject.

A CVD risk value, as well as its underlying VECTRA-CVD score, can be determined by assaying biomarker levels, and collecting clinical patient data for RA patients. The biomarker levels provide a score which can be combined with clinical data through application of an interpretation function to derive a CVD risk value, as well as its underlying VECTRA-CVD score.

In some aspects, a CVD risk value can be determined using a VECTRA-CVD score. A VECTRA-CVD score can be based on an Adjusted VECTRA score which can be combined with clinical parameters and additional adjustment terms for each of leptin, TNFRI, and MMP3 biomarkers. An Adjusted VECTRA score can be based on an MBDA score, which utilizes a number of VECTRA biomarkers, along with certain clinical variables and an additional adjustment term for leptin biomarker.

In some aspects, a quantitative measure of risk in a subject having inflammatory disease can be measured by determining the levels of two or more VECTRA biomarkers in a sample from the subject, then applying an interpretation function to transform the biomarker levels into an MBDA score. The MBDA score can be adjusted using certain clinical variables and an additional adjustment term for leptin biomarker, giving an Adjusted VECTRA score. The Adjusted VECTRA score can be combined with clinical parameters and additional adjustment terms for each of leptin, TNFRI, and MMP3 biomarkers to provide an overall VECTRA-CVD score. A CVD risk value for the subject can be derived from a VECTRA-CVD score.

A CVD risk value, as well as its underlying VECTRA-CVD score, can be determined in a training stage, followed by a validation stage using clinical patient data for RA patients.

A wide range of candidate variables can be assessed for use in determining a CVD risk value. A training and/or validation stage can determine the final variables to be used in determining a CVD risk value from among a wide range of candidate variables.

In a training stage, an interpretation function can be applied through univariate and bivariate analyses using clinical patient data for RA patients to provide a nexus between the CVD risk value, as well as its underlying VECTRA-CVD score, and clinical outcomes for the RA patients.

In a validation stage, an interpretation function can be applied through univariate and bivariate analyses using clinical patient data for RA patients to provide a validation of the CVD risk value, as well as its underlying VECTRA-CVD score, with respect to clinical outcomes for the RA patients. A validation of the CVD risk value can affirm the accuracy of the CVD risk value, as well as its underlying VECTRA-CVD score.

In the training stage and/or validation stage, the use or application of the interpretation function can determine the final variables to be used. An interpretation function can select the final variables to be used from a set of candidate variables. An interpretation function can select the weight to be applied to each of the selected variables.

In certain aspects, an MBDA score, derived as described herein, can be varied based on a set of values chosen by the practitioner. For example, a score can be set such that a value can be given a range from 0-100, and a difference between two scores would be a value of at least one point. The practitioner can then assign risk based on the values by establishing a nexus with patient data.

As used herein, the term “sample” can refer to a biological sample that can be isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including synovial fluid, gingival crevicular fluid, bone marrow, cerebrospinal fluid (CSF), saliva, mucous, sputum, semen, sweat, urine, or any other bodily fluids. The term “blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

As used herein, the term “subject” can be for example a human, or a mammal. The term “patient” can refer to a human patient, which may be a subject. The term “mammal” as used herein includes but is not limited to a human, non-human primate, dog, cat, mouse, rat, cow, horse, and pig. Mammals other than humans can be advantageously used as subjects that represent animal models of inflammation. A subject can be male or female. A subject can be one who has been previously diagnosed or identified as having an inflammatory disease. A subject can be one who has already undergone, or is undergoing, a therapeutic intervention for an inflammatory disease. A subject can also be one who has not been previously diagnosed as having an inflammatory disease; e.g., a subject can be one who exhibits one or more symptoms or risk factors for an inflammatory condition, or a subject who does not exhibit symptoms or risk factors for an inflammatory condition, or a subject who may be asymptomatic for inflammatory disease.

As used herein, the term “interpretation function” can refer to one or more biostatistical tools which can provide a predictive method for assessing patient risk and/or outcomes. A biostatistical tool can utilize patient clinical and/or biomarker data to identify or derive an accurate risk algorithm, which reflects a meaningful connection between patient outcomes and a risk value.

A biostatistical tool can generate a risk algorithm which accurately predicts a patient's disease risk based on clinical and/or biomarker data from a reference group.

A reference group may include patients having activity of a disease. In certain embodiments, a reference group may include patients having no activity of the disease.

In further embodiments, a reference group may include patients having activity of a disease, patients not having activity of a disease, and patients not diagnosed for a disease.

In certain embodiments, a reference group may include patients who have been tested for activity of a disease.

In additional embodiments, a reference group may include patients who have been tested for activity of rheumatoid arthritis (RA). The tests may have been performed as routine tests, as clinical study tests, as gene tests, or as MBDA tests.

In further embodiments, a reference group may include patients who have been tested for activity of rheumatoid arthritis (RA) and/or cardiovascular disease (CVD). The tests may have been performed as routine tests, as clinical study tests, as gene tests, or as MBDA tests.

In certain embodiments, a reference group may be Medicare patients.

In certain embodiments, a reference group may be patients of a health care database.

Interpretation functions may be known in the art for calculating and validating predictive clinical methods.

In additional embodiments, a reference group may be clinical training data and/or clinical validation data.

As used herein, the term “score” can refer to a value or set of values selected so as to provide a quantitative measure of a variable or characteristic of a subject's condition, and/or to discriminate, differentiate or otherwise characterize a subject's condition. The value(s) comprising the score can be based on, for example, quantitative data resulting in a measured amount of one or more sample constituents obtained from the subject, or from clinical parameters, or from clinical assessments, or any combination thereof. In certain embodiments the score can be derived from a single constituent, parameter or assessment, while in other embodiments the score can be derived from multiple constituents, parameters and/or assessments. A score can be determined using an interpretation function. A “change in score” can refer to the absolute change in score, e.g., from one time point to the next, or the percent change in score, or the change in the score per unit time (e.g., the rate of score change).

As used herein, the term “dataset” can refer to a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored.

As used herein, the term “difference” can refer to an increase or decrease.

As used herein, the term “statistically significant” can refer to an observed alteration greater than what would be expected to occur by chance alone. Statistical significance can be determined by any of various methods known in the art. An example of a commonly used measure of statistical significance is the p-value. A result may be considered statistically significant (not random chance) at a p-value less than or equal to 0.05, or in some embodiments a p-value less than or equal to 0.01. In general, p-value can be a measure of a probability of obtaining a result, and p-values can be compared for different results when taken in a similar context.

Methods of this invention contemplate the use of a “multi-biomarker disease activity score” (MBDA score). As used herein, the MBDA score can provide a quantitative measure of inflammatory disease activity or the state of inflammatory disease in a subject using various biomarkers. An interpretation function can be used according to the present teachings to derive the MBDA score using the biomarkers. Two or more biomarkers can be used, alone or in combination with clinical parameters and/or clinical assessments, also described herein. In some embodiments, an MBDA score can be a quantitative measure of autoimmune disease activity. In certain embodiments, an MBDA score may be a quantitative measure of RA disease activity.

Biomarkers that may be useful for deriving an MBDA score can include, but are not limited to: leptin (LEP), tumor necrosis factor receptor superfamily, member 1A (TNFRSF1A expressing TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA).

An MBDA score may be a clinically validated tool that quantifies 12 serum protein biomarkers to assess disease activity in adult patients with rheumatoid arthritis (RA). See Curtis J R, et al., Arthritis Care Res. 2012, Vol. 64, pp. 1794-803. The 12 MBDA serum protein biomarkers can be leptin, TNFR1, MMP-3, CRP, IL-6, SAA, YKL-40, EGF, VCAM-1, MMP-1, resistin, and VEGF-A.

A quantity called MBDA score “MDBA 1.0” was described in U.S. Pat. No. 9,200,324, which is hereby incorporated by reference in its entirety. “MBDA 2.0” or “adjusted MBDA” was described in Curtis et al., Rheumatology, Vol. 58(5), May 2019, pp. 874-883, which is hereby incorporated by reference in its entirety. Herein we refer to the “Adjusted MBDA” score as being the “Leptin Adjusted MBDA” score of Curtis et al. given by Formula I:

$$Adjusted MBDA score = MBDA score - 0.437 \times age + 3.31 \times sex + 0.0502 \times {LEP}^{0.58} - 0.0247 \times age \times sex - 0.000483 \times age \times {LEP}^{0.58} + 0.00254 \times sex \times {LEP}^{0.58} + 33. 9 \qquad Formula I$$

where sex is equal to 1 if the patient is male and 0 if female. The Adjusted MBDA score is rounded to the nearest integer. MBDA and Adjusted MBDA scores range from 1 to 100.

An Adjusted MBDA score of this disclosure may include biomarker data from three or more biomarkers of a set including, but not limited to, leptin, TNFR1, MMP-3, CRP, IL6, SAA, YKL40, EGF, VCAM1, MMP1, resistin, and VEGFA.

As used herein, the name of a gene, for example TNFRSF1A, refers also to the expressed protein marker, TNFR1, as well as the mRNA.

In some embodiments, this invention includes methods in which biomarkers levels are measured in a sample from a subject to generate protein level data for LEP, TNFR1, and MMP3 to be combined with an Adjusted MBDA score, along with clinical data.

In additional embodiments, this invention includes methods in which biomarkers are measured in a sample from a subject to generate protein level data for LEP, TNFR1, MMP3, and one or more additional protein markers which are selected from a set comprising CRP, IL6, SAA1, CHI3L1, EGF, VCAM1, MMP1, RETN, and VEGFA.

In additional embodiments, this invention includes methods in which biomarkers are measured in a sample from a subject to generate protein level data for LEP, TNFR1, and MMP3, which can be combined with clinical data including age, smoking, diabetes, hypertension, and/or history of CVD to determine CVD risk.

In additional embodiments, this invention includes methods in which biomarkers are measured in a sample from a subject to generate protein level data for LEP, TNFR1, MMP3, and one or more additional protein markers which are selected from a set comprising CRP, IL6, SAA1, CHI3L1, EGF, VCAM1, MMP1, RETN, and VEGFA, which can be combined with clinical data including age, smoking, diabetes, hypertension, and/or history of CVD to determine CVD risk.

In some embodiments, this invention includes methods in which biomarkers are measured in a sample from a subject to generate protein level data for one or more protein markers which are selected from a set comprising LEP, TNFRSF1A, MMP3, CRP, IL6, SAA1, CHI3L1, EGF, VCAM1, MMP1, RETN, and VEGFA.

In further embodiments, this invention includes methods in which biomarkers are measured in a sample from a subject to generate protein level data for at least two protein markers which are selected from a set comprising LEP, TNFRSF1A, MMP3, CRP, IL6, SAA1, CHI3L1, EGF, VCAM1, MMP1, RETN, and VEGFA.

In additional embodiments, this invention includes methods in which biomarkers are measured in a sample from a subject to generate protein level data for at least three protein markers which are selected from a set comprising LEP, TNFRSF1A, MMP3, CRP, IL6, SAA1, CHI3L1, EGF, VCAM1, MMP1, RETN, and VEGFA.

In some embodiments, this invention includes methods in which biomarkers are measured in a sample from a subject to generate protein level data for at least four protein markers which are selected from a set comprising LEP, TNFRSF1A, MMP3, CRP, IL6, SAA1, CHI3L1, EGF, VCAM1, MMP1, RETN, and VEGFA.

In further embodiments, this invention includes methods in which biomarkers are measured in a sample from a subject to generate protein level data for at least five protein markers which are selected from a set comprising LEP, TNFRSF1A, MMP3, CRP, IL6, SAA1, CHI3L1, EGF, VCAM1, MMP1, RETN, and VEGFA.

In certain embodiments, this invention includes methods in which biomarkers are measured in a sample from a subject to generate protein level data for at least six protein markers which are selected from a set comprising LEP, TNFRSF1A, MMP3, CRP, IL6, SAA1, CHI3L1, EGF, VCAM1, MMP1, RETN, and VEGFA.

Any of the above sets of biomarkers can be combined with clinical data including age, smoking, diabetes, hypertension, and/or history of CVD to determine CVD risk.

In general, population-based research in RA studying hard endpoints including myocardial infarction (MI) can be challenging because of the relatively low prevalence of RA and outcome event rates limits statistical power. In some embodiments, administrative data from health plans and payers can have high validity for studying large cohorts of patients with RA. While these data sources often lack clinical assessments of RA, results of lab tests that measure RA disease activity may provide objective measurements that can augment claims data.

As used herein, the term “performance” can refer to the quality and overall usefulness of, e.g., a model, algorithm, or diagnostic or prognostic test. Factors to be considered in model or test performance include, but are not limited to, the clinical and analytical accuracy of the test, use characteristics such as stability of reagents and various components, ease of use of the model or test, health or economic value, and relative costs of various reagents and components of the test. Performing can mean the act of carrying out a function.

As used herein, the term “population” can refer to a grouping of subjects of like specified characteristics. The grouping could be according to, for example but without limitation, clinical parameters, clinical assessments, therapeutic regimen, disease status (e.g. with disease or healthy), level of disease activity, and level of CVD risk. In the context of using the MBDA score in comparing risk between populations, an aggregate value can be determined based on the observed MBDA scores of the subjects of a population; e.g., at particular timepoints in a longitudinal study. The aggregate value can be based on, e.g., any mathematical or statistical formula useful and known in the art for arriving at a meaningful aggregate value from a collection of individual datapoints; e.g., mean, median, and median of the mean.

As used herein, the term “predictive model” can refer to a “multivariate model” or a “model,” which can be a mathematical construct developed using a statistical algorithm or algorithms for classifying sets of data. As used herein, the term “predicting” can refer to generating a value for a datapoint without actually performing the clinical diagnostic procedures normally or otherwise required to produce that datapoint. As used in a modeling context, “predicting” should not be understood solely to refer to the power of a model to predict a particular outcome. Predictive models can involve an interpretation function. For example, a predictive model can be created by utilizing one or more statistical tools or methodologies to transform a dataset of observed data into a prediction of a risk score, as related to a disease state of a subject.

As used herein, the term “prognosis” can refer to a prediction as to the likely outcome of a disease. Prognostic estimates are useful in, e.g., determining an appropriate therapeutic regimen for a subject.

As used herein, the term “recommending” can refer to making a recommendation for a therapeutic regimen or excluding (i.e., not recommending) a certain therapeutic regimen for a subject. Such a recommendation may serve together with other information as a basis for a clinician to apply a certain therapeutic regimen for an individual subject.

In some embodiments, an interpretation function may employ one or more of various biostatistical tools. Some biostatistical tools and methodologies are known in the art.

Examples of biostatistical tools include Survival Regression analysis, Cox Proportional Hazards, Box-Cox transformation, Clustering Machine Learning, Hierarchical Clustering Analysis, Centroid Clustering, Distribution Clustering, Density Clustering, Cluster Data Mining, analysis of variants (ANOVA), Ada-boosting, Classification and Regression Trees (CART), boosted CART, Random Forest (RF), Recursive Partitioning Trees (RPART), Curds and Whey (CW), Curds and Whey-Lasso, principal component analysis (PCA), factor rotation analysis, Linear Discriminant Analysis (LDA), Eigengene Linear Discriminant Analysis (ELDA), quadratic discriminant analysis, Discriminant Function Analysis (DFA), Hidden Markov Models, kernel density estimation, kernel partial least squares algorithm, kernel matching pursuit algorithm, kernel Fisher's discriminate analysis algorithm, kernel principal components analysis algorithm; linear regression, Stepwise Regression, Forward-Backward Variable Stepwise Regression, Lasso shrinkage and selection, Elastic Net regularization and selection, Lasso and Elastic Net-regularized generalized linear model, Logistic Regression (LogReg), Kth-nearest neighbor (KNN), non-linear regression, classification, neural networks, partial least square, rules based classification, shrunken centroids (SC), sliced inverse regression, Standard for the Exchange of Product model data, Application Interpreted Constructs (StepAIC), super principal component (SPC) regression, Support Vector Machines (SVM), and Recursive Support Vector Machines (RSVM), and combinations thereof, among others.

Additionally, clustering algorithms as are known in the art can be useful in determining subject sub-groups. Examples of clustering algorithms include Clustering Machine Learning, Hierarchical Clustering Analysis, Centroid Clustering, Distribution Clustering, Density Clustering, Cluster Data Mining, and combinations thereof.

Examples of biostatistical tools include Logistic Regression which can be used for dichotomous response variables; e.g., treatment 1 versus treatment 2. Logistic Regression can be used for both linear and non-linear aspects of the data variables and provides interpretable odds ratios.

Examples of biostatistical tools include Discriminant Function Analysis (DFA) which can use a set of analytes as variables (roots) to discriminate between two or more naturally occurring groups. DFA may be used to test analytes that are significantly different between groups. A forward step-wise DFA can be used to select a set of analytes that maximally discriminate among the groups studied. Specifically, at each step all variables can be reviewed to determine which will maximally discriminate among groups. This information may then be included in a discriminative function, denoted a root, which may be an equation consisting of linear combinations of analyte concentrations for the prediction of group membership. The discriminatory potential of the final equation can be observed as a line plot of the root values obtained for each group. This approach can identify groups of analytes whose changes in concentration levels can be used to delineate profiles, diagnose and assess therapeutic efficacy. The DFA model can also create an arbitrary score by which new subjects can be classified as either “healthy” or “diseased.” To facilitate the use of this score for the medical community the score can be rescaled so a value of 0 indicates a healthy individual and scores greater than 0 indicate increasing risk.

Examples of biostatistical tools include Classification and Regression Trees (CART) may perform logical splits (if/then) of data to create a decision tree. All observations that fall in a given node may be classified according to the most common outcome in that node. CART results can be interpreted by following a series of if/then tree branches until a classification results.

Examples of biostatistical tools include Support Vector Machines (SVM) may classify objects into two or more classes. Examples of classes include sets of treatment alternatives, sets of diagnostic alternatives, or sets of prognostic alternatives. Each object can be assigned to a class based on its similarity to (or distance from) objects in the training data set in which the correct class assignment of each object may be known. The measure of similarity of a new object to the known objects may be determined using support vectors, which define a region in a potentially high dimensional space (>R6).

Examples of biostatistical tools include methods used in this invention which may involve a process of bootstrap aggregating, or “bagging.” In a first step, a given dataset can be randomly resampled a specified number of times (e.g., thousands), effectively providing that number of new datasets, which may be referred to as “bootstrapped resamples” of data, each of which can then be used to build a model. Then, in the example of classification models, the class of every new observation may be predicted by the number of classification models created in the first step. The final class decision may be based upon a “majority vote” of the classification models; i.e., a final classification call can be determined by counting the number of times a new observation is classified into a given group, and taking the majority classification (33%+for a three-class system). In the example of logistical regression models, if a logistical regression is bagged 1000 times, there will be 1000 logistical models, and each will provide the probability of a sample belonging to class 1 or 2.

Examples of biostatistical tools include methods of this invention which may involve Curds and Whey (CW) using ordinary least squares (OLS) as a predictive modeling method. See L. Breiman and JH Friedman, J. Royal. Stat. Soc. B 1997, 59(1):3-54. This method can take advantage of the correlations between response variables to improve predictive accuracy, compared with the usual procedure of performing an individual regression of each response variable on the common set of predictor variables X. In CW, Y=XB*S, where Y=(ykj) with k for the k^{th} patient and j for j^{th} response (j=1 for TJC, j=2 for SJC, etc.), B can be obtained using OLS, and S can be the shrinkage matrix computed from the canonical coordinate system. Another method may be Curds and Whey and Lasso in combination (CW-Lasso). Instead of using OLS to obtain B, as in CW, here Lasso can be used, and parameters may be adjusted accordingly for the Lasso approach.

Examples of biostatistical tools include biomarker selection techniques (such as, for example, forward selection, backwards selection, or stepwise selection), which may be used in combination, or may be used for complete enumeration of all potential panels of a given size, or genetic algorithms, or they can themselves include biomarker selection methodologies in their own techniques. These techniques can be coupled with information criteria, such as Akaike's Information Criterion (AIC), B ayes Information Criterion (BIC), or cross-validation, to quantify the tradeoff between the inclusion of additional biomarkers and model improvement, and to minimize overfit. The resulting predictive models can be validated in other studies, or cross-validated in the study they were originally trained in, using such techniques as, for example, Leave-One-Out (LOO) and 10-Fold cross-validation (10-Fold CV).

As an example, an interpretation function may provide an MBDA score, derived using one or more biostatistical tools as described above, which can be represented by Formula II, referring to biomarkers one (BM1) through five (BM5):

MBDA=(BM1conc*(0.39\hat{ }0.5)+BM2conc*(0.39\hat{ }0.5)+BM3conc*(0.39\hat{ }0.5)+BM4conc*(0.36\hat{ }0.5)+BM5conc*(0.31\hat{ }0.5))/10 Formula II

An MBDA score obtained for RA subjects with known clinical assessments (e.g., DAS28 scores) can then be compared to those known assessments to determine the level of correlation between the two assessments, and hence determine the accuracy of the MBDA score and its underlying predictive model.

As an example, one or more steps of a method of this invention may be derived by one or more of the steps: obtaining biomarker data for a subject, selecting clinical parameters for combination with biomarker results, selecting additional biomarkers terms for combination with clinical parameters, using an interpretation function to calculate a risk score for a subject from one or more biomarkers and one or more clinical terms along with clinical training data, using one or more biostatistical tools to calculate and derive a risk score algorithm for a subject from biomarkers and one or more clinical terms along with clinical training data, using an interpretation function to calculate a risk score for a subject from biomarkers and one or more clinical terms along with clinical validation data, using one or more biostatistical tools to calculate and derive a risk score algorithm for a subject from biomarkers and one or more clinical terms along with clinical validation data, and/or applying a risk score algorithm to clinical and/or biomarker data from a subject in need.

As an example, one or more steps of a method of this invention may be using a risk score for assessing specific risk of cardiovascular disease in patients with an inflammatory disease, using a risk score for recommending specific therapy for a subject having an inflammatory disease, using a risk score for identifying a specific subject having an inflammatory disease and at risk of cardiovascular disease (CVD) who benefits from a treatment, using a risk score for treating cardiovascular disease (CVD) in a subject having an inflammatory disease and in need thereof, using a risk score for monitoring a specific response of a subject having an inflammatory disease and at risk of having CVD to a treatment, and using a risk score for prognosing a subject having an inflammatory disease and at risk of having CVD.

In some embodiments of the present teachings, it may not be required that the MBDA score be compared to any pre-determined “reference,” “normal,” “control,” “standard,” “healthy,” “pre-disease” or other like index, in order for the MBDA score to provide a quantitative measure of risk in the subject.

In further embodiments of the present teachings, the amount of the biomarker(s) can be measured in a sample and used to derive a MBDA score, which MBDA score may then be compared to a “normal” or “control” level or value, utilizing techniques such as, e.g., reference or discrimination limits or risk defining thresholds, in order to define cut-off points and/or abnormal values for CVD risk. The normal level may then be the level of one or more biomarkers or combined biomarker indices typically found in a subject who is not suffering from the inflammatory disease under evaluation. Other terms for “normal” or “control” are, e.g., “reference,” “index,” “baseline,” “standard,” “healthy,” and “pre-disease.” Such normal levels can vary, based on whether a biomarker is used alone or in a formula combined with other biomarkers to output a score. Alternatively, the normal level can be a database of biomarker patterns from previously tested subjects who did not convert to the inflammatory disease under evaluation over a clinically relevant time period. Reference (normal, control) values can also be derived from, e.g., a control subject or population whose risk is known.

In some embodiments of the present teachings, the reference value can be derived from one or more subjects who have been exposed to treatment for disease, or from one or more subjects who are at low risk, or from subjects who have shown improvements as a result of exposure to treatment. In some embodiments the reference value can be derived from one or more subjects who have not been exposed to treatment; for example, samples can be collected from (a) subjects who have received initial treatment, and (b) subjects who have received subsequent treatment, to monitor the progress of the treatment. A reference value can also be derived from risk algorithms or computed indices from population studies.

The terms “normal,” “control,” and “healthy,” as used herein, can refer generally to a subject or individual who does not have, is not/has not been diagnosed with, or is asymptomatic for a particular disease or disorder. The terms can also refer to a sample obtained from such subject or individual. The disease or disorder under analysis or comparison may be determinative of whether the subject is a “control” in that situation. By example, where the level of a particular serum marker is obtained from an individual known to have RA, but who is not diagnosed with and is asymptomatic for CVD, that subject can be the “RA subject.” The level of the marker thus obtained from the RA subject can be compared to the level of that same marker from a subject who is diagnosed with RA, but who is known not to have prevalent CVD and not to be a CVD progressor; i.e., a “normal subject.” Thus, “normal” in this example refers to the subject's CVD status, not RA status.

As used herein, the term “accuracy” can refer to the degree that a measured or calculated value conforms to its actual value. In clinical testing, “accuracy” may relate to the proportion of actual outcomes (true positives or true negatives, wherein a subject was correctly classified as having disease or as healthy/normal, respectively) versus incorrectly classified outcomes (false positives or false negatives, wherein a subject is incorrectly classified as having disease or as healthy/normal, respectively).

As used herein, the terms “sensitivity,” “specificity,” “positive predictive value (PPV),” “the AUC,” “negative predictive value (NPV),” “likelihood,” and “odds ratio” can refer to mathematical features which may reflect medical accuracy. “Analytical accuracy,” in the context of the present disclosure, can refer to the repeatability and predictability of the measurement process. Analytical accuracy can be summarized in such measurements as, e.g., coefficients of variation (CV), and tests of concordance and calibration of the same samples or controls at different times or with different assessors, users, equipment, and/or reagents. See, e.g., R. Vasan, Circulation 2006, 113(19):2335-2362.

In some embodiments, an MBDA test can be a panel of 12 biomarkers that have been validated against DAS28 in multiple RA cohorts consisting of seropositive and seronegative patients treated with a variety of RA therapies.

The biomarkers included in the MBDA can reflect the biology of RA and may include cytokines, acute phase reactants, growth factors, matrix metalloproteinases, and adipokines.

The 12 biomarkers can be weighted as a linear combination according to a published algorithm (Vectra 2.0) and produce a single score on a scale of 1-100.

For clinical interpretation, this numeric score can be mapped to RA disease activity categories of low, moderate, and high disease activity, or low, borderline, moderate, and high.

In addition to both the overall MBDA score and its component 12 biomarkers, various clinical parameters can be included in the prediction model based upon their expected association with CVD risk.

Candidate parameters for CVD risk can include age, sex, race, diagnoses and medications for diabetes, hypertension, hyperlipidemia, tobacco use, history of cardiovascular disease other than MI or stroke, e.g. angina or acute coronary syndrome without MI, atrial fibrillation, peripheral vascular disease, specific RA medications such as methotrexate, other conventional synthetic disease modifying anti-rheumatic drugs, biologics, janus kinase inhibitors, glucocorticoids, and non-steroidal anti-inflammatory use.

In additional aspects, this invention provides various methods for identifying, assaying, determining, and predicting CVD risk. The development of an accurate final therapeutic prognosis may require several phases of development, screening, and/or intermediate conclusions being drawn.

In some aspects, development of an accurate final therapeutic prognosis or prediction model may involve feature selection and model building using a training dataset.

In some embodiments, model building can involve selection conducted according to Transparent Reporting of a multivariable prediction model for Individual Prognosis or Diagnosis (TRIPOD) guidelines.

In further embodiments, a cohort may be randomly split 2:1 into separate training and test datasets. Feature selection and model building may be performed on the training data.

In additional embodiments, candidate features can be descriptively characterized.

In characterizing features, means (SD) can be calculated for continuous predictors and percentages can be calculated for categorical predictors, which may stratify patients according to whether or not they have CVD events.

In further aspects, a VECTRA-CVD score can include a hyperbolic-tangent function. A parameter for hyperbolic-tangent transformation may be chosen based on maximum likelihood estimation. A parameter for hyperbolic-tangent transformation may be updated in each step of model building.

In additional aspects, backward elimination with an alpha value of 0.05 can initially be used to screen features. Features may be prioritized based on their association with the outcome in univariate analysis.

In some embodiments, a final model may be selected by examining its performance as compared to models developed using only the training data. A number of factors can be used in selecting the final model including model discrimination, calibration, parsimony, and expected feasibility of collecting the required covariates in routine clinical care settings.

In certain embodiments, statistical analysis may be performed by risk categorization into various risk groups.

For example, in some embodiments, patients can be grouped into three risk groups by their predicted risk for CVD at three years.

In certain aspects, a unique algorithm can be developed for use as a CVD Risk Score.

In some embodiments, a method for obtaining a CVD Risk Score may have a step for calculating a VECTRA-CVD Score.

For example, a VECTRA-CVD Score can be calculated according to the Formula III:

$$VECTRA - CVD Score = + C 1 \times tanh ( Adjusted MBDA / 33.0807 ) + C 2 \times ln ( Leptin ) + C 3 \times ln ( T NFR 1 ) + C 4 \times ln ( MMP 3 ) + C 5 \times age + C 6 \times smoking + C 7 \times diabetes + C 8 \times hypertension + C 9 \times history of CVD \qquad Formula III$$

where Adjusted MBDA is a biomarker/clinical score as described above, which combines an MBDA score with clinical parameters age and sex, and further combines with an additional LEP adjustment term. Coefficients C1 to C9 can be determined by the multi-phase statistical model development and analysis interpretation function as described herein.

For “smoking,” smoking is 1 if the patient is designated as a smoker, 0 if not.

For “diabetes,” 1 if patient has diabetes, 0 if not.

For “hypertension,” 1 if patient has hypertension, 0 if not.

For “history of CVD,” 1 if patient has history of CVD, 0 if not.

In certain embodiments, in calculating the VECTRA-CVD Score, Leptin, TNFR1, and MMP3 refer to the serum concentrations of those biomarkers as measured in ng/mL, whereas, in calculating the Adjusted MBDA score from MBDA 2.0, the leptin concentration is measured in pg/mL.

In some embodiments, the coefficients C1 to C9 for a VECTRA CVD Score can be a range as shown in Table 1.

TABLE 1
Ranges for coefficients C1 to C9

| Variable | Coefficient | Lower limit | Upper limit |
|---|---|---|---|
| Inside coefficient for | 33.08073 | 32.92229 | 33.23917 |
| tanh (Adjusted | | | |
| VECTRA denominator) | | | |
| Outside coefficient C1 | 1.607582 | 0.805184 | 2.409979 |
| for tanh (Adjusted | | | |
| VECTRA) | | | |
| C2 Leptin | −0.171106 | −0.241418 | −0.100793 |
| C3 TNFR1 | 0.572441 | 0.359173 | 0.785710 |
| C4 MMP3 | 0.145355 | 0.030513 | 0.260197 |
| C5 Age | 0.031441 | 0.021760 | 0.041121 |
| C6 Smoking | 0.269117 | 0.087381 | 0.450852 |
| C7 Diabetes | 0.273186 | 0.107790 | 0.438582 |
| C8 Hypertension | 0.269370 | 0.005012 | 0.533727 |
| C9 History of CVD | 0.337822 | 0.167159 | 0.508486 |

In some embodiments, the Inside coefficient for tanh(Adjusted VECTRA denominator) can range from 32.92229 to 33.23917, preferably 33.08073±0.16.

In some embodiments, the Inside coefficient for tanh(Adjusted VECTRA denominator) can range from 32.9223 to 33.2392, preferably 33.0807±0.15.

In some embodiments, the Inside coefficient for tanh(Adjusted VECTRA denominator) can range from 32.922 to 33.239, preferably 33.081±0.15.

In some embodiments, the Inside coefficient for tanh(Adjusted VECTRA denominator) can range from 32.92 to 33.24, preferably 33.08±0.15.

In some embodiments, the Inside coefficient for tanh(Adjusted VECTRA denominator) can range from 32.9 to 33.2, preferably 33.1±0.2.

In some embodiments, C1 can be 0.80, or 0.90, or 1.00, or 1.10, or 1.20, or 1.30, or 1.40, or 1.50, or 1.60, or 1.70, or 1.80, or 1.90. or 2.00, or 2.10, or 2.20, or 2.30, or 2.41.

In some embodiments, C1 can range from 0.805184 to 2.409979, preferably 1.607582±0.8.

In some embodiments, C1 can be 1.61±0.80, or 1.607±0.802, or 1.6076±0.8024.

In some embodiments, C1 can be 0.8 to 2.4, preferably 1.6±0.8.

In some embodiments, C1 can be 0.80 to 2.41, preferably 1.61±0.8.

In some embodiments, C1 can be 0.805 to 2.410, preferably 1.607±0.8.

In some embodiments, C1 can be 0.8052 to 2.4100, preferably 1.6076±0.8.

In some embodiments, C2 can be −0.10, or −0.11, or −0.12, or −0.13, or −0.14, or −0.15, or −0.16, or −0.17, or −0.18, or −0.19, or −0.20, or −0.21, or −0.22, or −0.23, or −0.24.

In some embodiments, C2 can be −0.17±0.07, or −0.171±0.070, or −0.1711±0.0703.

In some embodiments, C2 can be −0.24 to −0.10, preferably −0.17±0.07.

In some embodiments, C2 can be −0.241 to −0.101, preferably −0.171±0.07.

In some embodiments, C2 can be −0.2414 to −0.1008, preferably −0.1711±0.07.

In some embodiments, C3 can be 0.36 to 0.78, or 0.359 to 0.786, or 0.3592 to 0.7857.

In some embodiments, C3 can be 0.57±0.21, or 0.572±0.213, or 0.5724±0.2133.

In some embodiments, C3 can be 0.36 to 0.78, preferably 0.57±0.2.

In some embodiments, C3 can be 0.359 to 0.786, preferably 0.572±0.2.

In some embodiments, C3 can be 0.3592 to 0.7857, preferably 0.5724±0.2.

In some embodiments, C4 can be 0.03 to 0.26, or 0.030 to 0.260, or 0.0304 to 0.2602.

In some embodiments, C4 can be 0.14±0.11, or 0.145±0.115, or 0.1453±0.1148.

In some embodiments, C4 can be 0.03 to 0.26, preferably 0.14±0.1.

In some embodiments, C4 can be 0.030 to 0.260, preferably 0.145±0.1.

In some embodiments, C4 can be 0.0305 to 0.2602, preferably 0.1453±0.1.

In some embodiments, C5 can be 0.02 to 0.04, or 0.021 to 0.041, or 0.0218 to 0.0411.

In some embodiments, C5 can be 0.03±0.01, or 0.031±0.010, or 0.0314±0.0097.

In some embodiments, C5 can be 0.02 to 0.04, preferably 0.03±0.01.

In some embodiments, C5 can be 0.022 to 0.041, preferably 0.031±0.01.

In some embodiments, C5 can be 0.0218 to 0.0411, preferably 0.0314±0.01.

In some embodiments, C6 can be 0.08 to 0.45, or 0.087 to 0.451, or 0.0874 to 0.4508.

In some embodiments, C6 can be 0.26±0.18, or 0.269±0.182, or 0.2691±0.1817.

In some embodiments, C6 can be 0.08 to 0.45, preferably 0.26±0.2.

In some embodiments, C6 can be 0.087 to 0.451, preferably 0.269±0.2.

In some embodiments, C6 can be 0.0874 to 0.4508, preferably 0.2691±0.2.

In some embodiments, C7 can be 0.10 to 0.44, or 0.108 to 0.438, or 0.1078 to 0.4386.

In some embodiments, C7 can be 0.27±0.16, or 0.273±0.165, or 0.2732±0.1654.

In some embodiments, C7 can be 0.11 to 0.44, preferably 0.27±0.2.

In some embodiments, C7 can be 0.108 to 0.438, preferably 0.273±0.2.

In some embodiments, C7 can be 0.1078 to 0.4386, preferably 0.2732±0.2.

In some embodiments, C8 can be 0.01 to 0.53, or 0.005 to 0.534, or 0.0050 to 0.5337.

In some embodiments, C8 can be 0.27±0.26, or 0.269±0.264, or 0.2694±0.2643.

In some embodiments, C8 can be 0.005 to 0.53, preferably 0.27±0.3.

In some embodiments, C8 can be 0.005 to 0.533, preferably 0.269±0.3.

In some embodiments, C8 can be 0.0050 to 0.5337, preferably 0.2694±0.3.

In some embodiments, C9 can be 0.17 to 0.51, or 0.167 to 0.508, or 0.1671 to 0.5084.

In some embodiments, C9 can be 0.34±0.17, or 0.338±0.167, or 0.3378±0.1671.

In some embodiments, C9 can be 0.17 to 0.51, preferably 0.34±0.2.

In some embodiments, C9 can be 0.167 to 0.508, preferably 0.338±0.2.

In some embodiments, C9 can be 0.1671 to 0.5085, preferably 0.3378±0.2.

In certain embodiments, to calculate a VECTRA CVD Risk Score when an individual marker term is missing, the missing term can be replaced by an average value obtained from a training set of data. For example, if the “smoking” data is not available, the term “+C6×smoking” can be replaced by an average value obtained from a training set of data.

In some embodiments, a final VECTRA-CVD Score for an RA patient can be calculated according to the Formula IV:

$$VECTRA - CVD Score = + 1.6 \; 076 \times tanh ( Adjusted MBDA / 33.0807 ) - 0.1711 \times ln ( Leptin ) + 0.5724 \times ln ( T NFR 1 ) + 0.1454 \times ln ( MMP 3 ) + 0.0314 \times age + 0.2691 \times smoking + 0.2732 \times diabetes + 0.2694 \times hypertension + 0.3378 \times history of CVD . \qquad Formula IV$$

In further embodiments, a VECTRA-CVD Score can be calculated according to the Formula V:

$$VECTRA - CVD Score = + 1.607582 \times tanh ( Adjusted MBDA / 33.08073 ) - 0.171106 \times ln ( Leptin ) + 0.572441 \times ln ( T NFR 1 ) + 0.145355 \times ln ( MMP 3 ) + 0.031441 \times age + 0.269117 \times smoking + 0.273186 \times diabetes + 0.26937 \times hypertension + 0.337822 \times history of CVD . \qquad Formula V$$

In some embodiments, an algorithm for a CVD Risk Score Predictor Test may have a step for converting the VECTRA-CVD Score into a percent risk.

In further embodiments, a VECTRA-CVD Score may be converted into a percent risk CVD Risk Score using Formula VI:

100×{1−A^{exp(B×VECTRA-CVD Score)}} Formula VI

where A reflects the baseline risk, which is the probability of no CVD event within 3 years when the VECTRA-CVD Score is zero, and B is the calibration constant applied to the testing dataset.

An interpretation function may provide an algorithm for a CVD score which includes a hyperbolic tangent or an exponential of a biomarker score. An interpretation function may provide an algorithm for a CVD score which is a VECTRA-CVD Score.

Examples of an interpretation function include Formula VI.

The baseline survival is estimated in the validation dataset from a Cox proportional hazards regression model where the VECTRA-CVD Score is the only predictor.

For example, in certain embodiments, A=0.9996, which is baseline survival, being the probability of no CVD event within 3 years when the VECTRA-CVD Score is zero, and B=1.0646, being an adjustment from training, which estimated coefficients, to validation, which estimated risk.

In additional embodiments, A=0.99963462142575399, which is baseline survival, being the probability of no CVD event within 3 years when the VECTRA-CVD Score is zero, and B=1.064597, being an adjustment from training, which estimated coefficients, to validation, which estimated risk.

In additional embodiments, ranges for quantities A and B in percent risk calculated from a VECTRA-CVD Score are shown in Table 2.

TABLE 2
Ranges for quantities A and B in percent
risk calculated from a VECTRA CVD Score

| Variable | Coefficient | Lower limit | Upper limit |
|---|---|---|---|
| A (baseline survival) | 0.9996346 | 0.9991987 | 0.9998334 |
| B (Risk score coefficient | 1.064597 | 0.902812 | 1.226382 |
| in the exponent) | | | |

In additional embodiments, the value for A on a ln(−ln(A)) scale can be −7.914394, with a range from −8.699876 to −7.128912.

In some embodiments, A can be or 0.9992 to 0.9998, or 0.99920 to 0.99983, or 0.999198 to 0.999833, or 0.9991987 to 0.9998334.

In some embodiments, A can be 0.9996±0.0003, or 0.99963±0.0003, or 0.999635±0.0003, or 0.9996346±0.0003.

In some embodiments, A can be 0.9992 to 0.9998, preferably 0.9996±0.0003.

In some embodiments, A can be 0.99920 to 0.99983, preferably 0.99963±0.0003.

In some embodiments, A can be 0.999199 to 0.999833, preferably 0.999635±0.0003.

In some embodiments, A can be 0.9991987 to 0.9998334, preferably 0.9996346±0.0003.

In some embodiments, B can be 0.90 to 1.23, or 0.903 to 1.226, or 0.9028 to 1.2264, or 0.90281 to 1.22638, or 0.902812 to 1.226382.

In some embodiments, B can be 1.06±0.16, or 1.064±0.162, or 1.0646±0.1618, or 1.06460±0.16178, or 1.064597±0.161785.

In some embodiments, B can be 0.90 to 1.23, preferably 1.06±0.2.

In some embodiments, B can be 0.903 to 1.226, preferably 1.064±0.2.

In some embodiments, B can be 0.9028 to 1.2264, preferably 1.0646±0.2.

For example, for an example patient with an Adjusted VECTRA score of 46, Age 70, leptin concentration of 3.7 ng/mL, MMP3 concentration of 21 ng/mL, TNFR1 concentration of 2.8 ng/mL, non-diabetic, smoker, with hypertension and a history of CVD, the CVD Risk Score is shown in Formula VII:

$$VECTRA - CVD Score = + 1.6 \; 076 \times tanh ( 46 / 33.0807 ) ( Adjusted MBDA ) - 0.1711 \times ln ( 3.7 ) [ Leptin ] + 0.5724 \times ln ( 2.8 ) [ TNFR 1 ] + 0.1454 \times ln ( 21 ) [ MMP 3 ] + 0.0314 \times 70 [ Age ] + 0.2691 \times 1 [ S moking ] + 0.2732 \times 0 [ Diabetes ] + 0.2694 \times 1 [ Hypertension ] + 0.3378 \times 1 [ History of CVD ] = 5.3052 \qquad Formula VII$$

This example patient's percent risk as a CVD 3-year Risk % is shown in Formula VIII:

$$100 \times \{ 1 - {0.999635}^{exp ( 1.0646 \times 5.3052 )} \} = 9.848 \% . \qquad Formula VIII$$

Embodiments of this invention further contemplate methods for assessing and treating CVD risk in RA by clinically validating the predictive ability of CVD Period Risk % values of this disclosure. Survival rates for RA patients can be used to validate three or four CVD risk group thresholds for the predictive CVD Period Risk % values of this invention.

In some embodiments, the thresholds can be low, intermediate, and high CVD risk. In further embodiments, the thresholds can be low, borderline, intermediate, and high CVD risk.

In further aspects, methods for assessing and treating CVD risk in RA by clinically validating the predictive ability of a CVD Period Risk % can involve a risk period over a wide range. In certain embodiments, the time period of a CVD Period Risk % can be from 1−20 years. In certain embodiments, a CVD Period Risk % can be a CVD 1-year Risk %, or a CVD 2-year Risk %, or a CVD 3-year Risk %, or a CVD 4-year Risk %, or a CVD 5-year Risk %, or a CVD 6-year Risk %, or a CVD 7-year Risk %, or a CVD 8-year Risk %, or a CVD 9-year Risk %, or a CVD 10-year Risk %. In certain embodiments, the time period of a CVD Period Risk % can be any time period, for example, based on hours, days, months, or years.

Methods disclosed herein can expand the population of patients encompassed for treatments. In some embodiments, this invention provides CVD 3-year Risk % values having surprisingly improved accuracy, which can allow patients to be reclassified as having higher risk than expected. Reclassified patients may receive treatment who would otherwise not have been treated. The population of patients encompassed for treatment can be expanded by methods of this invention which accurately assess risk of CVD in RA. The kind of treatments for CVD or RA applied to a population of patients can be modified due to the reclassification of patient risk in methods of this invention which accurately assess risk of CVD in RA.

In some embodiments, risk categories, which can be thresholds, for CVD 3-year Risk % of this invention can be validated from actual incidence in patient clinical outcomes.

In further embodiments, risk categories, which can be thresholds, for a CVD Risk % of this invention may take into account the 2019 ACC/AHA Guideline on the Primary Prevention of Cardiovascular Disease.

In certain embodiments, risk categories, which can be thresholds, for a CVD Risk % of this invention may take into account ten-year ACC/AHA thresholds of 5% (±0.1%), 7.5% (±0.1%), and 20% (±0.1%) risk. Thresholds may be selected based on cumulative risk.

Thresholds may be described as being low risk, borderline risk, intermediate risk, and high risk. In certain embodiments, low risk and borderline risk can be combined.

In some embodiments, a CVD 3-year Risk % of this invention can be used for reclassifying patients based on CVD risk in RA.

For the example of a three year time frame, thresholds may be selected for categorization.

In certain embodiments, the thresholds may be low risk (cumulative risk 0 to <1.8%), intermediate risk (1.8 to <5.2%), and high risk (>5.2%).

In certain embodiments, thresholds may correspond to the ten-year ACC/AHA thresholds of 7.5% (+−0.1%) and 20% (+−0.1%) risk.

In additional embodiments, thresholds may be selected as in any one of Tables 3-5.

TABLE 3
Thresholds for CVD Risk

| Category | 10-yr CVD Risk | 3-yr CVD Risk |
|---|---|---|
| Low-Borderline | <7.5% | <1.8% |
| Intermediate | ≥7.5% to <20% | ≥1.8% to <5.2% |
| High | ≥20% | ≥5.2% |

TABLE 4
Thresholds for CVD Risk

| Category | 10-yr CVD Risk | 3-yr CVD Risk |
|---|---|---|
| Low | <5% | <1.3% |
| Borderline | 5% to <7.5% | 1.3% to <1.8% |
| Intermediate | ≥7.5% to <20% | ≥1.8% to <5.2% |
| High | ≥20% | ≥5.2% |

TABLE 5
Thresholds for CVD Risk

| Category | 10-yr CVD Risk | 3-yr CVD Risk |
|---|---|---|
| Low | <5% | <1.3% |
| Borderline-Intermediate | >5% to <20% | ≥1.3% to <5.2% |
| High | ≥20% | ≥5.2% |

The quantity of one or more biomarkers of the present teachings can be indicated as a value. The value can be one or more numerical values resulting from the evaluation of a sample, and can be derived, e.g., by measuring level(s) of the biomarker(s) in a sample by an assay performed in a laboratory, or from dataset obtained from a provider such as a laboratory, or from a dataset stored on a server. Biomarker levels can be measured using any of several techniques known in the art. The present teachings encompass such techniques, and further include all subject fasting and/or temporal-based sampling procedures for measuring biomarkers.

“Biomarker,” “biomarkers,” “marker” or “markers” in the context of the present disclosure encompasses, without limitation, cytokines, chemokines, growth factors, proteins, peptides, nucleic acids, oligonucleotides, and metabolites, together with their related metabolites, mutations, isoforms, variants, polymorphisms, modifications, fragments, subunits, degradation products, elements, and other analytes or sample-derived measures. Biomarkers can also include mutated proteins, mutated nucleic acids, variations in copy numbers and/or transcript variants. Biomarkers also encompass non-blood borne factors and non-analyte physiological markers of health status, and/or other factors or markers not measured from samples (e.g., biological samples such as bodily fluids), such as clinical parameters and traditional factors for clinical assessments. Biomarkers can also include any indices that are calculated and/or created mathematically. Biomarkers can also include combinations of any one or more of the foregoing measurements, including temporal trends and differences. Where the biomarkers of certain embodiments of the present teachings are proteins, the gene symbols and names used herein are to be understood to refer to the protein products of these genes, and the protein products of these genes are intended to include any protein isoforms of these genes, whether or not such isoform sequences are specifically described herein. Where the biomarkers are nucleic acids, the gene symbols and names used herein are to refer to the nucleic acids (DNA or RNA) of these genes, and the nucleic acids of these genes are intended to include any transcript variants of these genes, whether or not such transcript variants are specifically described herein.

The term “cytokine” in the present teachings refers to any substance secreted by specific cells that can be of the immune system that carries signals between cells and thus has an effect on other cells. The term “cytokines” encompasses “growth factors.” “Chemokines” are also cytokines. They are a subset of cytokines that are able to induce chemotaxis in cells; thus, they are also known as “chemotactic cytokines.”

“Measuring” or “measurement” as well as “detecting” or “detection” in the context of the present disclosure refers to determining the presence, absence, quantity, amount, or effective amount of a substance in a clinical or subject-derived sample, including the concentration levels of such substances, or evaluating the values or categorization of a subject's clinical parameters.

A “multiplex assay” as used herein refers to an assay that simultaneously measures multiple analytes, e.g., protein analytes, in a single run or cycle of the assay.

A “quantitative dataset” or “quantitative data” as used in the present teachings, refers to the data derived from, e.g., detection and composite measurements of expression of a plurality of biomarkers (i.e., two or more) in a subject sample. The quantitative dataset can be used to generate a score for the identification, monitoring and treatment of disease states, and in characterizing the biological condition of a subject. It is possible that different biomarkers will be detected depending on the disease state or physiological condition of interest.

The term “analyte” in the context of the present disclosure can mean any substance to be measured, and can encompass biomarkers, markers, nucleic acids, electrolytes, metabolites, proteins, sugars, carbohydrates, fats, lipids, cytokines, chemokines, growth factors, proteins, peptides, nucleic acids, oligonucleotides, metabolites, mutations, variants, polymorphisms, modifications, fragments, subunits, degradation products and other elements. For simplicity, standard gene symbols may be used throughout to refer not only to genes but also gene products/proteins, rather than using the standard protein symbol; e.g., APOA1 as used herein can refer to the gene APOA1 and also the protein ApoAI. In general, hyphens have been omitted from analyte names and symbols herein (IL-6=IL6).

To “analyze” includes determining a value or set of values associated with a sample by measurement of analyte levels in the sample. “Analyze” may further comprise comparing the levels against constituent levels in a sample or set of samples from the same subject or other subject(s), including reference levels. The biomarkers of the present teachings can be analyzed by any of various methods in the art. Some such methods include but are not limited to: measuring serum protein or sugar or metabolite or other analyte level, measuring enzymatic activity, and measuring gene expression. Some techniques include immunoassays, mass spectrometry, etc. An “immunoassay” as used herein refers to a biochemical assay that uses one or more antibodies to measure the presence or concentration of an analyte or biomarker in a biological sample.

The actual measurement of levels of the biomarkers can be determined at the protein or nucleic acid level using any method known in the art. “Protein” detection comprises detection of full-length proteins, mature proteins, pre-proteins, polypeptides, isoforms, mutations, variants, post-translationally modified proteins and variants thereof, and can be detected in any suitable manner. Levels of biomarkers can be determined at the protein level, e.g., by measuring the serum levels of peptides encoded by the gene products described herein, or by measuring the enzymatic activities of these protein biomarkers. Such methods are well-known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the biomarker genes according to the activity of each protein analyzed. For biomarker proteins, polypeptides, isoforms, mutations, and variants thereof known to have enzymatic activity, the activities can be determined in vitro using enzyme assays known in the art. Such assays include, without limitation, protease assays, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

The term “antibody” refers to any immunoglobulin-like molecule that reversibly binds to another with the required selectivity. Thus, the term includes any such molecule that is capable of selectively binding to a biomarker of the present teachings. The term includes an immunoglobulin molecule capable of binding an epitope present on an antigen. The term is intended to encompass not only intact immunoglobulin molecules, such as monoclonal and polyclonal antibodies, but also antibody isotypes, recombinant antibodies, bi-specific antibodies, humanized antibodies, chimeric antibodies, anti-idiopathic (anti-ID) antibodies, single-chain antibodies, Fab fragments, F(ab′) fragments, fusion protein antibody fragments, immunoglobulin fragments, Fy fragments, single chain Fy fragments, and chimeras comprising an immunoglobulin sequence and any modifications of the foregoing that comprise an antigen recognition site of the required selectivity.

Using sequence information provided by the public database entries for the biomarker, expression of the biomarker can be detected and measured using techniques well-known to those of skill in the art. For example, nucleic acid sequences in the sequence databases that correspond to nucleic acids of biomarkers can be used to construct primers and probes for detecting and/or measuring biomarker nucleic acids. These probes can be used in, e.g., Northern or Southern blot hybridization analyses, ribonuclease protection assays, and/or methods that quantitatively amplify specific nucleic acid sequences. As another example, sequences from sequence databases can be used to construct primers for specifically amplifying biomarker sequences in, e.g., amplification-based detection and quantitation methods such as reverse-transcription based polymerase chain reaction (RT-PCR) and PCR. When alterations in gene expression are associated with gene amplification, nucleotide deletions, polymorphisms, post-translational modifications and/or mutations, sequence comparisons in test and reference populations can be made by comparing relative amounts of the examined DNA sequences in the test and reference populations.

As an example, Northern hybridization analysis using probes which specifically recognize one or more of these sequences can be used to determine gene expression. Alternatively, expression can be measured using RT-PCR; e.g., polynucleotide primers specific for the differentially expressed biomarker mRNA sequences reverse-transcribe the mRNA into DNA, which is then amplified in PCR and can be visualized and quantified. Biomarker RNA can also be quantified using, for example, other target amplification methods, such as TMA, SDA, and NASBA, or signal amplification methods (e.g., bDNA), and the like. Ribonuclease protection assays can also be used, using probes that specifically recognize one or more biomarker mRNA sequences, to determine gene expression.

Alternatively, biomarker protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. See, for example, WO 04/056456 and WO 04/088309, each of which is hereby incorporated by reference in its entirety. In this regard, other biomarker analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other biomarker metabolites can be similarly detected using reagents that are specifically designed or tailored to detect such metabolites.

In some embodiments, a biomarker is detected by contacting a subject sample with reagents, generating complexes of reagent and analyte, and detecting the complexes. Examples of “reagents” include but are not limited to nucleic acid primers and antibodies.

In some embodiments of the present teachings an antibody binding assay is used to detect a biomarker; e.g., a sample from the subject is contacted with an antibody reagent that binds the biomarker analyte, a reaction product (or complex) comprising the antibody reagent and analyte is generated, and the presence (or absence) or amount of the complex is determined. The antibody reagent useful in detecting biomarker analytes can be monoclonal, polyclonal, chimeric, recombinant, or a fragment of the foregoing, as discussed in detail above, and the step of detecting the reaction product can be carried out with any suitable immunoassay. The sample from the subject is typically a biological fluid as described above, and can be the same sample of biological fluid as is used to conduct the method described above.

Immunoassays carried out in accordance with the present teachings can be homogeneous assays or heterogeneous assays. Immunoassays carried out in accordance with the present teachings can be multiplexed. In a homogeneous assay the immunological reaction can involve the specific antibody (e.g., anti-biomarker protein antibody), a labeled analyte, and the sample of interest. The label produces a signal, and the signal arising from the label becomes modified, directly or indirectly, upon binding of the labeled analyte to the antibody. Both the immunological reaction of binding, and detection of the extent of binding, can be carried out in a homogeneous solution. Immunochemical labels which can be employed include but are not limited to free radicals, radioisotopes, fluorescent dyes, enzymes, bacteriophages, and coenzymes. Immunoassays include competition assays.

In a heterogeneous assay approach, the reagents can be the sample of interest, an antibody, and a reagent for producing a detectable signal. Samples as described above can be used. The antibody can be immobilized on a support, such as a bead (such as protein A and protein G agarose beads), plate or slide, and contacted with the sample suspected of containing the biomarker in liquid phase. The support is separated from the liquid phase, and either the support phase or the liquid phase is examined using methods known in the art for detecting signal. The signal is related to the presence of the analyte in the sample. Methods for producing a detectable signal include but are not limited to the use of radioactive labels, fluorescent labels, or enzyme labels. For example, if the antigen to be detected contains a second binding site, an antibody which binds to that site can be conjugated to a detectable (signal-generating) group and added to the liquid phase reaction solution before the separation step. The presence of the detectable group on the solid support indicates the presence of the biomarker in the test sample. Examples of suitable immunoassays include but are not limited to oligonucleotides, immunoblotting, immunoprecipitation, immunofluorescence methods, chemiluminescence methods, electrochemiluminescence (ECL), and/or enzyme-linked immunoassays (ELISA).

Those skilled in the art will be familiar with numerous specific immunoassay formats and variations thereof which can be useful for carrying out the method disclosed herein. See, e.g., E. Maggio, Enzyme-Immunoassay (1980), CRC Press, Inc., Boca Raton, FL. See also U.S. Pat. No. 4,727,022 to C. Skold et al., titled “Novel Methods for Modulating Ligand-Receptor Interactions and their Application”; U.S. Pat. No. 4,659,678 to GC Forrest et al., titled “Immunoassay of Antigens”; U.S. Pat. No. 4,376,110 to GS David et al., titled “Immunometric Assays Using Monoclonal Antibodies”; U.S. Pat. No. 4,275,149 to D. Litman et al., titled “Macromolecular Environment Control in Specific Receptor Assays”; U.S. Pat. No. 4,233,402 to E. Maggio et al., titled “Reagents and Method Employing Channeling”; and, U.S. Pat. No. 4,230,797 to R. Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein can likewise be conjugated to detectable labels or groups such as radiolabels (e.g., 35S, 125I, 131I), enzyme labels (e.g., horseradish peroxidase, alkaline phosphatase), and fluorescent labels (e.g., fluorescein, Alexa, green fluorescent protein, rhodamine) in accordance with known techniques.

Antibodies may also be useful for detecting post-translational modifications of biomarkers. Examples of post-translational modifications include, but are not limited to tyrosine phosphorylation, threonine phosphorylation, serine phosphorylation, citrullination and glycosylation (e.g., O-GlcNAc). Such antibodies specifically detect the phosphorylated amino acids in a protein or proteins of interest, and can be used in the immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF). See U. Wirth et al., Proteomics 2002, 2(10):1445-1451.

This invention can provide methods for recommending a therapeutic regimen, including withdrawal, tapering or cessation from a therapeutic regimen. Methods for recommending a therapeutic regimen can follow the determination of differences in expression of the biomarkers disclosed herein. Methods for recommending a therapeutic regimen can be based on the determination of a CVD Risk Score of this invention.

Measuring scores derived from expression levels of the biomarkers disclosed herein over a period time can provide a clinician with a dynamic picture of a subject's biological state. These embodiments of the present teachings thus will provide subject-specific biological information, which will be informative for therapy decision and will facilitate therapy response monitoring, and should result in more rapid and more optimized treatment, better control of disease, and an increase in the proportion of subjects achieving remission.

A “biologic” or “biotherapy” or “biopharmaceutical” may be a pharmaceutical therapy product manufactured or extracted from a biological substance. A biologic can include vaccines, blood or blood components, allergenics, somatic cells, gene therapies, tissues, recombinant proteins, and living cells; and can be composed of sugars, proteins, nucleic acids, living cells or tissues, or combinations thereof. Examples of biologic drugs can include but are not limited to biological agents that target the tumor necrosis factor (TNF)-alpha molecules and the TNF inhibitors, such as infliximab, adalimumab, etanercept and golimumab. Other classes of biologic drugs include IL1 inhibitors such as anakinra, T-cell modulators such as abatacept, B-cell modulators such as rituximab, and IL6 inhibitors such as tocilizumab.

Treatment strategies for CVD for patients with autoimmune disorders are confounded by the fact that some autoimmune disorders, such as RA, may be a classification given to a group of subjects with a diverse array of related symptoms that can flare or go into remission. This suggests that certain subtypes of RA are driven by specific cell type or cytokine. As a likely consequence, no single therapy has proven optimal for treatment. Given the increasing numbers of therapeutic options available, the need for an individually tailored treatment directed by immunological prognostic factors of treatment outcome may be needed.

The term “flare” as used herein may refer to a sudden and severe increase in the onset of symptoms and clinical manifestations including, but not limited to, an increase in SJC, increase in TJC, increase in serologic markers of inflammation (e.g., CRP and ESR), decrease in subject function (e.g., ability to perform basic daily activities), increase in morning stiffness, and increases in pain that commonly lead to therapeutic intervention and potentially to treatment intensification.

In some embodiments, prediction of CVD risk, in particular in RA patients, who can successfully withdrawal from or discontinue therapy, can be based on a MBDA score. In some embodiments, a high MBDA score as described herein at baseline can be an independent predictor of risk within a certain period of time following discontinuation of therapy. In some embodiments, a moderate MBDA score as described herein at baseline can be an independent predictor of risk within a certain period of time following discontinuation of therapy. In some embodiments, a low MBDA score as described herein at baseline can be an independent predictor of risk, or remission, within a certain period of time following discontinuation of therapy.

A “therapeutic regimen,” “therapy” or “treatment(s),” as described herein, includes all clinical management of a subject and interventions, whether biological, chemical, physical, or a combination thereof, intended to sustain, ameliorate, improve, or otherwise alter the condition of a subject. These terms may be used synonymously herein. Treatments include but are not limited to administration of prophylactics or therapeutic compounds (including conventional DMARDs, biologic DMARDs, non-steroidal anti-inflammatory drugs (NSAID's) such as COX-2 selective inhibitors, and corticosteroids), exercise regimens, physical therapy, dietary modification and/or supplementation, bariatric surgical intervention, administration of pharmaceuticals and/or anti-inflammatories (prescription or over-the-counter), and any other treatments known in the art as efficacious in preventing, delaying the onset of, or ameliorating disease. A “response to treatment” includes a subject's response to any of the above-described treatments, whether biological, chemical, physical, or a combination of the foregoing. A “treatment course” relates to the dosage, duration, extent, etc. of a particular treatment or therapeutic regimen. An initial therapeutic regimen as used herein may be the first line of treatment.

A DMARD can be conventional or biologic. Examples of DMARDs that are generally considered conventional include, but are not limited to, MTX, azathioprine (AZA), bucillamine (BUC), chloroquine (CQ), ciclosporin (CSA, or cyclosporine, or cyclosporin), doxycycline (DOXY), hydroxychloroquine (HCQ), intramuscular gold (IM gold), leflunomide (LEF), levofloxacin (LEV), and sulfasalazine (SSZ). Examples of other conventional DMARDs include, but are not limited to, folinic acid, D-pencillamine, gold auranofin, gold aurothioglucose, gold thiomalate, cyclophosphamide, and chlorambucil. Examples of biologic DMARDs (or biologic drugs) include but are not limited to biological agents that target the tumor necrosis factor (TNF)-alpha molecules such as infliximab, adalimumab, etanercept and golimumab. Other classes of biologic DMARDs include IL1 inhibitors such as anakinra, T-cell modulators such as abatacept, B-cell modulators such as rituximab, and IL6 inhibitors such as tocilizumab.

Treatment strategies for CVD for patients with autoimmune disorders may include one or more of the drugs in Table 6.

TABLE 6
Generic names of drugs

| Kind | Generic name |
|---|---|
| Statin | atorvastatin, cerivastatin, fluvastatin, gemfibrozil, |
| | lovastatin, pitavastatin, pravastatin, rosuvastatin, |
| | simvastatin |
| Other lipid lowering | cholestyramine, clofibrate, colesevelam, colestipol, |
| drug | dextrothyroxine, ezetimibe, fenofibrate, fenofibric, |
| | gemfibrozil, lomitapide, mipomersen, niacin |
| Ace inhibitor | benazepril, captopril, enalapril, fosinopril, lisinopril, moexipril, |
| | perindopril, quinapril, ramipril, trandolapril |
| Calcium channel | amlodipine, bepridil, clevidipine, diltiazem, felodipine, isradipine, |
| blockers | mibefradil, nicardipine, nifedipine, nimodipine, nisoldipine, |
| | verapamil |
| Thiazides and | bendroflumethiazide, chlorothiazide, chlorthalidone, |
| combinations | hydrochlorothiazide, hydroflumethiazide, indapamide, |
| | methyclothiazide, metolazone, polythiazide, trichlormethiazide, hctz |
| Diuretics potassium | amiloride, triamterene |
| sparing | |
| Diuretics, Loop | bumetanide, ethacrynate, ethacrynic acid, furosemide, torsemide |
| Aldosterone receptor | eplerenone, spironolactone |
| blockers | |
| Alpha blockers | doxazosin, prazosin, terazosin |
| ARBs | azilsartan, candesartan, eprosartan, irbesartan, losartan, |
| | olmesartan, telmisartan, valsartan |
| Renin | aliskiren |
| Beta blocker | acebutolol, atenolol, betaxolol, bisoprolol, carteolol, carvedilol, |
| | esmolol, labetalol, metoprolol, nadolol, nebivolol, penbutolol, |
| | pindolol, ′propranolol, sotalol, timolol |
| Central acting | clonidine, guanabenz, guanfacine, methyldopa, methyldopate |
| hypertension agents | |
| Diuretics | diazoxide, hydralazine, minoxidil, nitroprusside sodium, tolazoline |
| NSAIDS | bromfenac, celecoxib, diclofenac, diflunisal, etodola, fenoprofen, |
| | flurbiprofen, ibuprofen, indomethacin, ketoprofen, ketorolac, |
| | meclofenamate, mefenamic, meloxicam nabumetone, naproxen, |
| | oxaprozin, piroxicam, rofecoxib, salicylate, salsalate, sulindac, |
| | tolmetin, valdecoxib |
| TNFi biologic DMARDS | adalimumab, certolizumab, etanercept, golimumab, infliximab |
| Non-TNF biologic | abatacept, rituximab, tocilizumab, tofacitinib, sarilumab, baricitinib |
| DMARDS and targeted | |
| therapies | |
| Methotrexate | methotrexate |
| Other csDMARDS | hydroxychloroquine, leflunomide, sulfasalazine |
| besides methotrexate | |

A “time point” as used herein refers to a manner of describing a time, which can be substantially described with a single point. A time point may also be described as a time range of a minimal unit which can be detected. A time point can refer to a state of the aspect of a time or a manner of description of a certain period of time. Such a time point or range can include, for example, an order of seconds, minutes to hours, or days.

In some embodiments, the levels of one or more analyte biomarkers or the levels of a specific panel of analyte biomarkers in a sample are compared to a reference standard (“reference standard,” or “reference level,” or “control”) in order to direct treatment decisions. Expression levels of the one or more biomarkers can be combined into a score, which can represent risk.

The reference standard used for any embodiment disclosed herein may comprise average, mean, or median levels of the one or more analyte biomarkers or the levels of the specific panel of analyte biomarkers in a control population.

The reference standard may further include an earlier time point for the same subject. For example, a reference standard may include a first time point, and the levels of the one or more analyte biomarkers can be examined again at second, third, fourth, fifth, sixth time points, etc. Any time point earlier than any particular time point can be considered a reference standard.

The reference standard may additionally comprise cutoff values or any other statistical attribute of the control population, or earlier time points of the same subject, such as a standard deviation from the mean levels of the one or more analyte biomarkers or the levels of the specific panel of analyte biomarkers.

In some embodiments, the control population may comprise healthy individuals or the same subject prior to the administration of any therapy.

A reference standard may be inherently reflected in the clinical training and/or validation data used in a method of this disclosure.

In some embodiments, a score may be obtained from the reference time point, and a different score may be obtained from a later time point. A first time point can be when an initial therapeutic regimen is begun. A first time point can also be when a first immunoassay is performed. A time point can be hours, days, months, years, etc. In some embodiments, a time point is one month. In some embodiments, a time point is two months. In some embodiments, a time point is three months. In some embodiments, a time point is four months. In some embodiments, a time point is five months. In some embodiments, a time point is six months. In some embodiments, a time point is seven months. In some embodiments, a time point is eight months. In some embodiments, a time point is nine months. In some embodiments, a time point is ten months. In some embodiments, a time point is eleven months. In some embodiments, a time point is twelve months. In some embodiments, a time point is two years. In some embodiments, a time point is three years. In some embodiments, a time point is four years. In some embodiments, a time point is five years. In some embodiments, a time point is ten years.

A difference in the score can be interpreted as a decrease in risk. For example, lower score can indicate a lower level of risk. In these circumstances a second score having a lower score than the reference score, or first score, means that the subject's risk has been lowered (improved) between the first and second time periods. Alternatively, a higher score can indicate a lower level of risk. In these circumstances, a second score having a higher score than the reference score, or first score, also means that the subject's risk has improved between the first and second time periods.

A difference in the score can also be interpreted as an increase in risk. For example, lower score can indicate a higher level of risk. In these circumstances a second score having a lower score than the reference score, or first score, means that the subject's risk has been increased (worsened) between the first and second time periods. Alternatively, a higher score can indicate a higher level of risk. In these circumstances, a second score having a higher score than the reference score, or first score, also means that the subject's risk has worsened between the first and second time periods.

The differences can be variable. For example, when a difference in the score is interpreted as a decrease in risk, a large difference can mean a greater decrease in risk than a lower or moderate difference. Alternatively, when a difference in the score is interpreted as an increase in risk, a large difference can mean a greater increase in risk than a lower or moderate difference.

In some embodiments, a patient may be treated more or less aggressively than a reference therapy based on the difference of scores. A reference therapy is any therapy that is the standard of care for treatment. The standard of care can vary temporally and geographically, and a skilled person can easily determine the appropriate standard of care by consulting the relevant medical literature.

In further embodiments, a more aggressive therapy than the standard therapy comprises beginning treatment earlier than in the standard therapy. In some embodiments, a more aggressive therapy than the standard therapy comprises administering additional treatments than in the standard therapy. In some embodiments, a more aggressive therapy than the standard therapy comprises treating on an accelerated schedule compared to the standard therapy. In some embodiments, a more aggressive therapy than the standard therapy comprises administering additional treatments not called for in the standard therapy.

In additional embodiments, a less aggressive therapy than the standard therapy comprises delaying treatment relative to the standard therapy. In some embodiments, a less aggressive therapy than the standard therapy comprises administering less treatment than in the standard therapy. In some embodiments, a less aggressive therapy than the standard therapy comprises administering treatment on a decelerated schedule compared to the standard therapy. In some embodiments, a less aggressive therapy than the standard therapy comprises administering no treatment.

In some embodiments, a practitioner may discontinue a therapy regimen if a score is low. In certain embodiments, a practitioner may not change the therapy regimen if the score is high.

In one embodiment, the practitioner adjusts the therapy based on a comparison between difference scores, or based on an initial predictive score. In one embodiment, the practitioner adjusts the therapy by selecting and administering a different drug. In one embodiment, the practitioner adjusts the therapy by selecting and administering a different combination of drugs.

In one embodiment, the practitioner adjusts the therapy by adjusting drug dosage. In one embodiment, the practitioner adjusts the therapy by adjusting dose schedule. In one embodiment, the practitioner adjusts the therapy by adjusting length of therapy. In one embodiment, the practitioner adjusts the therapy by selecting and administering a different drug combination and adjusting drug dosage.

In one embodiment, the practitioner adjusts the therapy by selecting and administering a different drug combination and adjusting dose schedule. In one embodiment, the practitioner adjusts the therapy by selecting and administering a different drug combination and adjusting length of therapy. In one embodiment, the practitioner adjusts the therapy by adjusting drug dosage and dose schedule. In one embodiment, the practitioner adjusts the therapy by adjusting drug dosage and adjusting length of therapy.

In one embodiment, the practitioner adjusts the therapy by adjusting dose schedule and adjusting length of therapy. In one embodiment, the practitioner adjusts the therapy by selecting and administering a different drug, adjusting drug dosage, and adjusting dose schedule. In one embodiment, the practitioner adjusts the therapy by selecting and administering a different drug, adjusting drug dosage, and adjusting length of therapy.

In one embodiment, the practitioner adjusts the therapy by selecting and administering a different drug, adjusting dose schedule, and adjusting length of therapy. In one embodiment, the practitioner adjusts the therapy by adjusting drug dosage, adjusting dose schedule, and adjusting length of therapy. In one embodiment, the practitioner adjusts the therapy by selecting and administering a different drug, adjusting drug dosage, adjusting dose schedule, and adjusting length of therapy.

In one embodiment a less aggressive therapy comprises no change in the therapy regimen. In one embodiment a less aggressive therapy comprises delaying treatment. In one embodiment a less aggressive therapy comprises selecting and administering less potent drugs. In one embodiment a less aggressive therapy comprises decreasing the frequency treatment. In one embodiment a less aggressive therapy comprises shortening length of therapy.

In one embodiment, less aggressive therapy comprises selecting and administering less potent drugs and decreasing drug dosage. In one embodiment, less aggressive therapy comprises selecting and administering less potent drugs and decelerating dose schedule. In one embodiment, less aggressive therapy comprises selecting and administering less potent drugs and shortening length of therapy.

In one embodiment, less aggressive therapy comprises decreasing drug dosage and decelerating dose schedule. In one embodiment, less aggressive therapy comprises decreasing drug dosage and shortening length of therapy. In one embodiment, less aggressive therapy comprises decelerating dose schedule and shortening length of therapy.

In one embodiment, less aggressive therapy comprises selecting and administering less potent drugs, decreasing drug dosage, and decelerating dose schedule. In one embodiment, less aggressive therapy comprises selecting and administering less potent drugs, decreasing drug dosage, and shortening length of therapy. In one embodiment, less aggressive therapy comprises selecting and administering less potent drugs, decelerating dose schedule, and shortening length of therapy. In one embodiment, less aggressive therapy comprises decreasing drug dosage, decelerating dose schedule, and shortening length of therapy. In one embodiment, less aggressive therapy comprises selecting and administering less potent drugs, decreasing drug dosage, decelerating dose schedule, and shortening length of therapy. In some embodiments, a less aggressive therapy comprises administering only non-drug-based therapies.

In another aspect of the present document, treatment comprises a more aggressive therapy than a reference therapy. In one embodiment a more aggressive therapy comprises increased length of therapy. In one embodiment a more aggressive therapy comprises increased frequency of the dose schedule. In one embodiment, more aggressive therapy comprises selecting and administering more potent drugs and increasing drug dosage.

In one embodiment, more aggressive therapy comprises selecting and administering more potent drugs and accelerating dose schedule. In one embodiment, more aggressive therapy comprises selecting and administering more potent drugs and increasing length of therapy. In one embodiment, more aggressive therapy comprises increasing drug dosage and accelerating dose schedule. In one embodiment, more aggressive therapy comprises increasing drug dosage and increasing length of therapy. In one embodiment, more aggressive therapy comprises accelerating dose schedule and increasing length of therapy.

In one embodiment, more aggressive therapy comprises selecting and administering more potent drugs, increasing drug dosage, and accelerating dose schedule. In one embodiment, more aggressive therapy comprises selecting and administering more potent drugs, increasing drug dosage, and increasing length of therapy. In one embodiment, more aggressive therapy comprises selecting and administering more potent drugs, accelerating dose schedule, and increasing length of therapy. In one embodiment, more aggressive therapy comprises increasing drug dosage, accelerating dose schedule, and increasing length of therapy. In one embodiment, more aggressive therapy comprises selecting and administering more potent drugs, increasing drug dosage, accelerating dose schedule, and increasing length of therapy. In some embodiments, a more aggressive therapy comprises administering a combination of drug-based therapies, non-drug-based therapies, or a combination of classes of drug-based therapies.

In some embodiments of the present disclosure, MBDA scores are tailored to the population, endpoints or clinical assessment, and/or use that is intended. For example, a MBDA score can be used to assess subjects for primary prevention and diagnosis, and for secondary prevention and management. For the primary assessment, the MBDA score can be used for prediction and risk stratification for future conditions or disease sequelae, for the diagnosis of inflammatory disease and CVD risk, for the prognosis of disease activity and rate of change, and for indications for future diagnosis and therapeutic regimens. For secondary prevention and clinical management, the MBDA score can be used for prognosis and risk stratification. The MBDA score can be used for clinical decision support, such as determining whether to defer intervention or treatment, to recommend preventive check-ups for at-risk patients, to recommend increased visit frequency, to recommend increased testing, and to recommend intervention. The MBDA score can also be useful for therapeutic selection, determining response to treatment, adjustment and dosing of treatment, monitoring ongoing therapeutic efficiency, monitoring therapy withdrawal, and indication for change in therapeutic regimen.

In some embodiments of the present teachings, the MBDA score can be used to aid in the diagnosis of inflammatory disease and predict CVD risk, and in the determination of the severity of inflammatory disease. The MBDA score can also be used for determining the future status of intervention such as, for example in RA, determining the prognosis of future joint erosion with or without treatment or CVD risk with or without treatment. Certain embodiments of the present teachings can be tailored to a specific treatment or a combination of treatments.

Clinical variables that can be used to adjust the MBDA score can include, for example, gender/sex, smoking status, age, race/ethnicity, disease duration, diastolic and systolic blood pressure, resting heart rate, height, weight, adiposity, body-mass index, serum leptin, family history, CCP status (i.e., whether subject is positive or negative for anti-CCP antibody), CCP titer, RF status, RF titer, ESR, CRP titer, menopausal status, and whether a smoker/non-smoker.

The term “clinical variable” or “clinical parameter” in the context of the present disclosure encompasses all measures of the health or physiological status of a subject. A clinical parameter can be used to derive a clinical assessment of the subject's disease activity. Clinical parameters can include, without limitation: therapeutic regimen (including but not limited to DMARDs, whether conventional or biologics, steroids, etc.), TJC, SJC, morning stiffness, arthritis of three or more joint areas, arthritis of hand joints, symmetric arthritis, rheumatoid nodules, radiographic changes and other imaging, gender/sex, smoking status, age, race/ethnicity, disease duration, diastolic and systolic blood pressure, resting heart rate, height, weight, adiposity, body-mass index, serum leptin, family history, CCP status (i.e., whether subject is positive or negative for anti-CCP antibody), CCP titer, RF status, RF titer, ESR, CRP titer, menopausal status, and whether a smoker/non-smoker.

“Clinical assessment” and “clinical parameter” are not mutually exclusive terms. There may be overlap in members of the two categories. For example, CRP concentration can be used as a clinical assessment of disease activity; or, it can be used as a measure of the health status of a subject, and thus serve as a clinical parameter.

A “clinical assessment,” or “clinical datapoint” or “clinical endpoint,” in the context of the present teachings can refer to a measure of disease activity or severity. A clinical assessment can include a score, a value, or a set of values that can be obtained from evaluation of a sample (or population of samples) from a subject or subjects under determined conditions. A clinical assessment can also be a questionnaire completed by a subject. A clinical assessment can also be predicted by biomarkers and/or other parameters. One of skill in the art will recognize that the clinical assessment for RA, as an example, can comprise, without limitation, one or more of the following: DAS (defined herein), DAS28, DAS28-ESR, DAS28-CRP, health assessment questionnaire (HAQ), modified HAQ (mHAQ), multi-dimensional HAQ (MDHAQ), visual analog scale (VAS), physician global assessment VAS, patient global assessment VAS, pain VAS, fatigue VAS, overall VAS, sleep VAS, simplified disease activity index (SDAI), clinical disease activity index (CDAI), routine assessment of patient index data (RAPID), RAPID3, RAPID4, RAPID5, American College of Rheumatology (ACR), ACR20, ACR50, ACR70, SF-36 (a well-validated measure of general health status), RA MRI score (RAMRIS; or RA MRI scoring system), total Sharp score (TSS), van der Heijde-modified TSS, van der Heijde-modified Sharp score (or Sharp-van der Heijde score (SHS)), Larsen score, TJC, swollen joint count (SJC), CRP titer (or level), and erythrocyte sedimentation rate (ESR).

“DAS” refers to the Disease Activity Score, a measure of the activity of RA in a subject, well-known to those of skill in the art. See D. van der Heijde et al., Ann. Rheum. Dis. 1990, 49(11):916-920. “DAS” as used herein refers to this particular Disease Activity Score. The “DAS28” involves the evaluation of 28 specific joints. It can be a current standard well-recognized in research and clinical practice. Because the DAS28 can be a well-recognized standard, it may be referred to as “DAS.” Although “DAS” may refer to calculations based on 66/68 or 44 joint counts, unless otherwise specified, “DAS” herein will encompass the DAS28. Unless otherwise specified herein, the term “DAS28,” as used in the present teachings, can refer to a DAS28-ESR or DAS28-CRP, as obtained by any of the four formulas described above; or, DAS28 can refer to another reliable DAS28 formula as may be known in the art.

A DAS28 can be calculated for an RA subject according to the standard as outlined at the das-score.nl website, maintained by the Department of Rheumatology of the University Medical Centre in Nijmegen, the Netherlands. The number of swollen joints, or swollen joint count out of a total of 28 (SJC28), and tender joints, or tender joint count out of a total of 28 (TJC28) in each subject is assessed. In some DAS28 calculations the subject's general health (GH) is also a factor, and can be measured on a 100 mm Visual Analogue Scale (VAS). GH may also be referred to herein as PG or PGA, for “patient global health assessment” (or merely “patient global assessment”). A “patient global health assessment VAS,” then, is GH measured on a Visual Analogue Scale.

“DAS28-CRP” (or “DAS28CRP”) is a DAS28 assessment calculated using CRP in place of ESR (see below). CRP is produced in the liver. Normally there is little or no CRP circulating in an individual's blood serum— CRP is generally present in the body during episodes of acute inflammation or infection, so that a high or increasing amount of CRP in blood serum can be associated with acute infection or inflammation. A blood serum level of CRP greater than 1 mg/dL is usually considered high. Most inflammation and infections result in CRP levels greater than 10 mg/dL. The amount of CRP in subject sera can be quantified using, for example, the DSL-10-42100 ACTIVE® US C-Reactive Protein Enzyme-Linked Immunosorbent Assay (ELISA), developed by Diagnostics Systems Laboratories, Inc. (Webster, TX). CRP production is associated with radiological progression in RA. See M. Van Leeuwen et al., Br. J. Rheum. 1993, 32(suppl.):9-13). CRP is thus considered an appropriate alternative to ESR in measuring RA disease activity. See R. Mallya et al., J. Rheum. 1982, 9(2):224-228, and F. Wolfe, J. Rheum. 1997, 24:1477-1485.

The DAS28-CRP can be calculated according to either of the formulas below, with or without the GH factor, where “CRP” represents the amount of this protein present in a subject's blood serum in mg/L, “sqrt” represents the square root, and “ln” represents the natural logarithm:
(a) DAS28-CRP with GH (or DAS28-CRP4)=(0.56*sqrt(TJC28)+0.28*sqrt(SJC28)+0.36*ln(CRP+1))+(0.014*GH)+0.96; or,
(b) DAS28-CRP without GH (or DAS28-CRP3)=(0.56*sqrt(TJC28)+0.28*sqrt(SJC28)+0.36*ln(CRP+1))*1.10+1.15.

The “DAS28-ESR” is a DAS28 assessment wherein the ESR for each subject is also measured (in mm/hour). The DAS28-ESR can be calculated according to the formula:
(a) DAS28-ESR with GH (or DAS28-ESR4)=0.56*sqrt(TJC28)+0.28*sqrt(SJC28)+0.70*ln(ESR)+0.014*GH; or,
(b) DAS28-ESR without GH=0.56*sqrt(TJC28)+0.28*sqrt(SJC28)+0.70*ln(ESR)*1.08+0.16.

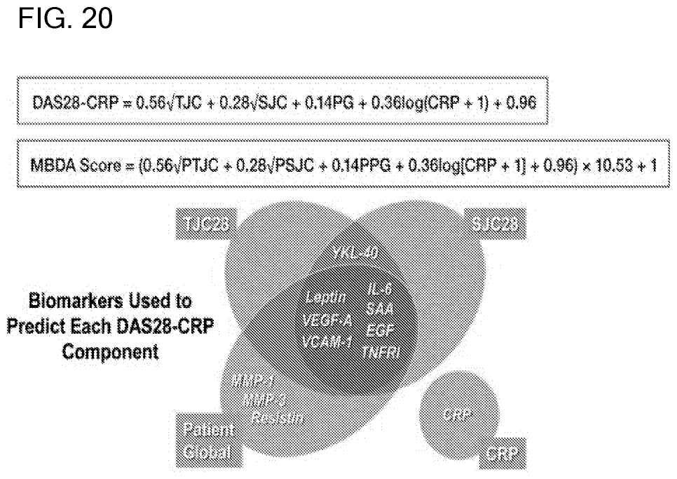

FIG. 20 shows biomarkers used to predict each DAS28-CRP component.

Tests for measuring risk according to various embodiments of the present teachings can be implemented on a variety of systems typically used for obtaining test results, such as results from immunological or nucleic acid detection assays. Such systems may comprise modules that automate sample preparation, that automate testing such as measuring biomarker levels, that facilitate testing of multiple samples, and/or are programmed to assay the same test or different tests on each sample. In some embodiments, the testing system comprises one or more of a sample preparation module, a clinical chemistry module, and an immunoassay module on one platform. Testing systems are typically designed such that they also comprise modules to collect, store, and track results, such as by connecting to and utilizing a database residing on hardware. Examples of these modules include physical and electronic data storage devices as are well-known in the art, such as a hard drive, flash memory, and magnetic tape. Test systems also generally comprise a module for reporting and/or visualizing results. Some examples of reporting modules include a visible display or graphical user interface, links to a database, or a printer.

The term “computer” carries the meaning that is generally known in the art; that is, a machine for manipulating data according to a set of instructions. As used herein, the term “module” refers to computer program logic utilized to provide the specified functionality. Thus, a module can be implemented in hardware, firmware, and/or software. Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

In some embodiments, this invention can comprise a system for determining CVD risk of a subject. In some embodiments, the system employs a module for applying a formula to an input comprising the measured levels of biomarkers in a panel, as described herein, and outputting a score. In some embodiments, the measured biomarker levels are test results, which serve as inputs to a computer that is programmed to apply the formula. The system may comprise other inputs in addition to or in combination with biomarker results in order to derive an output score; e.g., one or more clinical parameters such as therapeutic regimen, TJC, SJC, morning stiffness, arthritis of three or more joint areas, arthritis of hand joints, symmetric arthritis, rheumatoid nodules, radiographic changes and other imaging, gender/sex, age, race/ethnicity, disease duration, height, weight, body-mass index, family history, CCP status, RF status, ESR, smoker/non-smoker, etc. In some embodiments the system can apply a formula to biomarker level inputs, and then output a risk score that can then be analyzed in conjunction with other inputs such as other clinical parameters. In other embodiments, the system is designed to apply a formula to the biomarker and non-biomarker inputs (such as clinical parameters) together, and then report a composite output risk index.

A number of testing systems are presently available that could be used to implement various embodiments of the present teachings. See, for example, the ARCHITECT series of integrated immunochemistry systems—high-throughput, automated, clinical chemistry analyzers (ARCHITECT is a registered trademark of Abbott Laboratories, Abbott Park, Ill. 60064). See C. Wilson et al., “Clinical Chemistry Analyzer Sub-System Level Performance,” American Association for Clinical Chemistry Annual Meeting, Chicago, Ill., Jul. 23-27, 2006; and, H J Kisner, “Product development: the making of the Abbott ARCHITECT,” Clin. Lab. Manage. Rev. 1997 Nov.-Dec., 11(6):419-21; A. Ognibene et al., “A new modular chemiluminescence immunoassay analyzer evaluated,” Clin. Chem. Lab. Med. 2000 March, 38(3):251-60; J W Park et al., “Three-year experience in using total laboratory automation system,” Southeast Asian J. Trop. Med. Public Health 2002, 33 Suppl 2:68−73; D. Pauli et al., “The Abbott Architect c8000: analytical performance and productivity characteristics of a new analyzer applied to general chemistry testing,” Clin. Lab. 2005, 51(1-2):31-41.

Another testing system useful for embodiments of the present teachings is the VITROS system (VITROS, Johnson & Johnson Corp., New Brunswick, NJ) an apparatus for chemistry analysis that is used to generate test results from blood and other body fluids for laboratories and clinics. Another testing system is the DIMENSION system (DIMENSION, Dade Behring Inc., Deerfield Ill.) a system for the analysis of body fluids, comprising computer software and hardware for operating the analyzers, and analyzing the data generated by the analyzers.

The testing required for various embodiments of the present teachings, e.g. measuring biomarker levels, can be performed by laboratories such as those certified under the Clinical Laboratory Improvement Amendments (42 U.S.C. Section 263(a)), or by laboratories certified under any other federal or state law, or the law of any other country, state or province that governs the operation of laboratories that analyze samples for clinical purposes. Such laboratories include, for example, Laboratory Corporation of America, 358 South Main Street, Burlington, NC 27215 (corporate headquarters); Quest Diagnostics, 3 Giralda Farms, Madison, NJ 07940 (corporate headquarters); and other reference and clinical chemistry laboratories.

Additional embodiments of this invention include biomarker detection reagents packaged together in the form of a kit for conducting any of the assays of the present teachings. In certain embodiments, the kits comprise oligonucleotides that specifically identify one or more biomarker nucleic acids based on homology and/or complementarity with biomarker nucleic acids. The oligonucleotide sequences may correspond to fragments of the biomarker nucleic acids. For example, the oligonucleotides can be more than 200, 200, 150, 100, 50, 25, 10, or fewer than 10 nucleotides in length. In other embodiments, the kits comprise antibodies to proteins encoded by the biomarker nucleic acids. The kits of the present teachings can also comprise aptamers. The kit can contain in separate containers a nucleic acid or antibody (the antibody either bound to a solid matrix, or packaged separately with reagents for binding to a matrix), control formulations (positive and/or negative), and/or a detectable label, such as but not limited to fluorescein, green fluorescent protein, rhodamine, cyanine dyes, Alexa dyes, luciferase, and radiolabels, among others. Instructions for carrying out the assay, including, optionally, instructions for generating a MBDA score, can be included in the kit; e.g., written, tape, VCR, or CD-ROM. The assay can for example be in the form of a Northern hybridization or a sandwich ELISA as known in the art.

In some embodiments of the present teachings, biomarker detection reagents can be immobilized on a solid matrix, such as a porous strip, to form at least one biomarker detection site. In some embodiments, the measurement or detection region of the porous strip can include a plurality of sites containing a nucleic acid. In some embodiments, the test strip can also contain sites for negative and/or positive controls. Alternatively, control sites can be located on a separate strip from the test strip. Optionally, the different detection sites can contain different amounts of immobilized nucleic acids, e.g., a higher amount in the first detection site and lesser amounts in subsequent sites. Upon the addition of test sample, the number of sites displaying a detectable signal provides a quantitative indication of the amount of biomarker present in the sample. The detection sites can be configured in any suitably detectable shape and can be, e.g., in the shape of a bar or dot spanning the width of a test strip.

In other embodiments of the present teachings, the kit can contain a nucleic acid substrate array comprising one or more nucleic acid sequences. The nucleic acids on the array specifically identify one or more nucleic acid sequences represented by the MBDA markers. In various embodiments, the expression of one or more of the sequences represented by the MBDA markers can be identified by virtue of binding to the array. In some embodiments the substrate array can be on a solid substrate, such as what is known as a “chip.” See, e.g., U.S. Pat. No. 5,744,305. In some embodiments the substrate array can be a solution array; e.g., xMAP (Luminex, Austin, TX), Cyvera (Illumina, San Diego, CA), RayBio Antibody Arrays (RayBiotech, Inc., Norcross, GA), CellCard (Vitra Bioscience, Mountain View, CA) and Quantum Dots' Mosaic (Invitrogen, Carlsbad, CA).

A machine-readable storage medium can comprise, for example, a data storage material that is encoded with machine-readable data or data arrays. The data and machine-readable storage medium are capable of being used for a variety of purposes, when using a machine programmed with instructions for using said data. Such purposes include, without limitation, storing, accessing and manipulating information relating to the risk of a subject or population over time, or risk in response to treatment, or for drug discovery for inflammatory disease, etc. Data comprising measurements of the biomarkers of the present teachings, and/or the evaluation of CVD risk from these biomarkers, can be implemented in computer programs that are executing on programmable computers, which comprise a processor, a data storage system, one or more input devices, one or more output devices, etc. Program code can be applied to the input data to perform the functions described herein, and to generate output information. This output information can then be applied to one or more output devices, according to methods well-known in the art. The computer can be, for example, a personal computer, a microcomputer, or a workstation of conventional design.

The computer programs can be implemented in a high-level procedural or object-oriented programming language, to communicate with a computer system. The programs can also be implemented in machine or assembly language. The programming language can also be a compiled or interpreted language. Each computer program can be stored on storage media or a device such as ROM, magnetic diskette, etc., and can be readable by a programmable computer for configuring and operating the computer when the storage media or device is read by the computer to perform the described procedures. Any health-related data management systems of the present teachings can be considered to be implemented as a computer-readable storage medium, configured with a computer program, where the storage medium causes a computer to operate in a specific manner to perform various functions, as described herein.

The biomarkers disclosed herein can be used to generate a “subject biomarker profile” taken from subjects who have inflammatory disease. The subject biomarker profiles can then be compared to a reference biomarker profile, in order to diagnose or identify subjects with increased CVD risk. The biomarker profiles, reference and subject, of embodiments of the present teachings can be contained in a machine-readable medium, such as analog tapes like those readable by a CD-ROM or USB flash media, among others. Such machine-readable media can also contain additional test results, such as measurements of clinical parameters and clinical assessments. The machine-readable media can also comprise subject information; e.g., the subject's medical or family history. The machine-readable media can also contain information relating to other disease activity algorithms and computed scores or indices, such as those described herein.

The practice of the present teachings may employ methods of protein chemistry, biochemistry, recombinant DNA techniques, and pharmacology. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, PA; Carey and Sundberg, Advanced Organic Chemistry, Vols. A and B, 3rd Edition, 1992, Plenum Press.

The practice of the present teachings may employ methods of biostatistical analysis. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, NY; W. Cooley and P. Lohnes, Multivariate procedures for the behavioral science 1962, John Wiley and Sons, Inc. NY; E. Jackson, A User's Guide to Principal Components 2003, John Wiley and Sons, Inc., NY.

All publications, patents and literature specifically mentioned herein are hereby incorporated by reference in their entirety for all purposes.

Words specifically defined herein have the meaning provided in the context of the present disclosure as a whole, and as are typically understood by those skilled in the art. As used herein, the singular forms “a,” “an,” and “the” include the plural.

While the present disclosure is described in conjunction with various embodiments, it is not intended that the present disclosure be limited to such embodiments. On the contrary, the present disclosure encompasses various alternatives, modifications, and equivalents, as will be appreciated by those of skill in the art.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples herein are illustrative only and not intended to be limiting.

Although the foregoing disclosure has been described in some detail by way of illustration and examples for purposes of clarity of understanding, it will be understood by persons of skill in the art that various changes and modifications may be practiced within the scope of the invention and the appended claims.

### EXAMPLES

### Example 1: A VECTRA Biomarker Cardiovascular Disease Score for Rheumatoid Arthritis Patients

A retrospective RA cohort was created using the data source 100% sample of fee-for-service Medicare data 2011-2016 of individuals who were underwent testing with the commercially available MBDA test between 2001 and 2016. Data were linked on patient's date of birth, sex, and MBDA testing codes defined by procedure codes (Current Procedural Terminology codes 81479, 83520, 84999, 86140 and 81490, submitted by Crescendo Biosciences or Myriad Genetics) and MBDA test date, and treating rheumatologist's national provider identifier (NPI). Data were linked deterministically, using methods previously published. The UAB institutional review board approved the study.

Eligible Participants. Eligible patients were required to
1) be age >=40 years;
2) have at least one RA diagnosis code (ICD9 714.0; ICD10 M05.*, M06.*, excluding M06.4 and M06.1) from a rheumatologist;
3) receive RA-specific treatment (e.g. biologics, tumor necrosis factor inhibitors, abatacept, rituximab, IL-6Rs, janus kinase inhibitors, conventional synthetic disease-modifying anti-rheumatic drugs including methotrexate, sulfasalazine, leflunomide, and hydroxychloroquine etc.) prescribed anytime up to and including the date of the first MBDA test; and
4) at least one linked MBDA lab test.

Participants were also required to have at least at least 365 days of continuous coverage with Medicare parts A (hospital coverage), part B (outpatient coverage) and part D (pharmacy coverage).

The accuracy of this claims-based algorithm to identify RA exceeded 85%, and was further increased by the linkage with MBDA testing, given that the MBDA test was only approved for use in patients who were known to have RA (i.e. it was not a screening test to help diagnose RA).

Baseline was anchored at the first MBDA test date and included all available preceding Medicare data; a minimum of one year was required. Patients were excluded if they had any diagnosis code for malignancy (other than non-melanoma skin cancer), prior MI or stroke in the baseline period. Follow-up could potentially begin on the date of the first MBDA test after all eligibility requirements had been met. Follow-up ended at the earliest of the CVD outcome, the occurrence of malignancy, non-fatal CVD event (initially treated as a competing risk, later removed as it had minimal effect on results), or the end of study (12/31/2016)

CVD Outcome. The composite CVD outcome included hospitalized myocardial infarction (MI), stroke and fatal CVD. MI was defined as ICD-9 diagnosis code 410.x1 or ICD-10 diagnosis code I21.*(presented any single digit, with * representing any digits of number or characters) from an inpatient hospitalization that lasted at least one night in the hospital, or where the patient died. Stroke was identified using ICD-9 diagnosis codes 430.*, 431.*, 433.x1, 434.x1, 436.* or ICD-10 diagnosis code I60.*, I61.*, I63.* or 167.89 from hospital discharge. The positive predictive value of MI using this approach was approximately 93% or better and for stroke is approximately 80−85% (18-20). Fatal CVD was identified using published algorithms with PPV >=80%.

MBDA biomarker score. The VECTRA MBDA test was a panel of 12 protein biomarkers that have been previously and repeatedly validated against the DAS28-CRP in multiple RA cohorts consisting of seropositive and seronegative patients treated with a variety of RA therapies. The biomarkers included in the MBDA reflected the biology of RA and consist of cytokines (IL-6, TNFRI), acute phase reactants (serum amyloid A, C reactive protein), growth factors (VCAM-1, EGF, VEGF-A), matrix metalloproteinases (MMP-1, MMP-3), and adipokines (resistin, leptin). The 12 biomarkers were weighted in a published algorithm that was developed and validated based on correlation of the MBDA score with DAS28-CRP. Prior to and independently of the present study, an algorithm was developed to adjust the MBDA score for the effects of age, sex and leptin (as a surrogate for adiposity). Like the original MBDA score, this adjusted MBDA score had a scale of 1-100 and RA disease activity categories of low (<30), moderate (30-44), and high (>44). This adjustment has been in routine use since December 2017. MBDA scores were converted to adjusted MBDA scores for use in this study. As used herein, the term “MBDA score” refers to the adjusted MBDA score. All MBDA scores were ordered by practitioners as part of routine care and run in a commercial laboratory.

Individual MBDA test results were ignored if they fell within 14 days following any hospital discharge. MBDA test results were also ignored if the patient used sarilumab or tocilizumab in the preceding 90 days, given that IL-6R treatment influences the MBDA score in a way that might confound CVD risk prediction.

In addition to both the MBDA score and its component 12 biomarkers, which were log transformed, a variety of demographic and clinical predictors also were considered for inclusion in the prediction model based upon their expected association with CVD risk, informed by subject matter expertise and the medical literature. Candidate predictors included age, sex, race, diagnoses and medications for diabetes, hypertension, hyperlipidemia, tobacco use, history of cardiovascular disease other than MI or stroke [e.g. angina or acute coronary syndrome without MI, atrial fibrillation, peripheral vascular disease], RA medications as described above, glucocorticoids and non-steroidal anti-inflammatory use). All candidate predictors except the biomarkers were obtained using in Medicare data. Lab results, other than the MBDA test result and its component biomarkers, were not available in Medicare data.

Based upon a pre-specified analysis plan, a principled approach to model building and selection was conducted that followed Transparent Reporting of a multivariable prediction model for Individual Prognosis or Diagnosis (TRIPOD) guidelines. First, the cohort was randomly split 2:1 into separate training and testing (i.e. internal validation) datasets. All feature selection and model building was performed only on the training dataset.

Secondly, all candidate features were descriptively characterized. Predictors considered for model building included diagnosis codes of established CVD risk factors, demographic variables, and biomarker variables. Means (SD) were calculated for continuous predictors and percentages were calculated for categorical predictors, stratifying patients by whether or not they had a CVD event.

In building the statistical model, which is a part of a method of this invention, patient risk of CVD was estimated as a function of the predictors using Cox proportional hazards regression on only the training data. Individual biomarker concentrations were natural log-transformed. A squared term for age was included as a candidate predictor to account for possible non-linearity. The MBDA scores were hyperbolic-tangent transformed, with a parameter for the hyperbolic-tangent transformation chosen based on maximum likelihood estimation and updated in each step of model building. Consistent with one of the options described in the TRIPOD guidance, the model was built using backwards elimination: in the first step, every candidate predictor was included; in each subsequent step, the least significant variable (highest p-value) was dropped, and the model was refit with the remaining variables. This was done until all remaining variables had p-values below a threshold of 0.05.

A total of five prediction methods were compared using the training dataset. These five methods were:
1) age+sex;
2) age+sex+CRP;
3) a parsimonious clinical model (age+sex+tobacco use+diabetes+hypertension+history of other CVD conditions);
4) a parsimonious clinical model (#3 above) plus CRP; and
5) the final VECTRA-CVD Score MBDA-based method.

The considerations in selecting the final VECTRA-CVD Score method were based on model discrimination, calibration, parsimony, and the expected feasibility of collecting the required covariates in routine clinical care settings.

In this large cohort of more than 30,000 RA patients, a biomarker-based CVD prediction model was derived and validated for use in RA patients. In additional to reflecting the contribution of systemic inflammation as measured by the MBDA, the method incorporated 4 clinical predictors that should be readily available to clinicians at the point of care. The model was shown to be accurate in both the group of patients used to derived it, as well as a randomly split sample used for internal validation, and across a variety of key patient subgroups. The goal with such a risk assessment tool was to reflect the important contribution that active RA has on mediating elevated CVD risk and to bring a useful tool to rheumatologists and their patients at the point of care to facilitate shared decision-making. These discussions can cover topics including how to mitigate the contribution of RA disease activity and also highlight traditional CV risk factors that may need to be addressed with patient.

The validated CVD risk prediction score and methods of this invention can provide clinicians an easy-to-use measure to assess CVD risk at the point of care. The methods are based on age and a few readily-available traditional CVD risk factors combined with RA-related disease activity based on serum biomarkers. The methods of this invention emphasize actionable risk CVD factors, RA-related disease activity, as well as tobacco use and obesity (e.g. represented by the leptin biomarker) although other traditional CVD risk factors such as hypertension and diabetes are also included.

### Example 2: Risk Categorization for a VECTRA Biomarker Cardiovascular

Disease Score for Rheumatoid Arthritis Patients. A VECTRA Biomarker CVD 3-year Risk % Determination for Rheumatoid Arthritis Patients.

Patients in the training dataset were grouped into three risk groups by their predicted risk for CVD at three years. The 3-year estimated risk was calculated as 100×(1-Aexp[B×VECTRA-CVD Score]), where A reflects the baseline risk i.e. the probability of no CVD event within 3 years setting all covariates to 0, and B is the calibration constant applied to the testing dataset (see below). The 3-year time frame was chosen based on the availability of the data. For the purpose of categorizing risk values, 3-year cutpoints corresponding to the ten-year ACC/AHA thresholds of 5% (+−0.1%), 7.5% (+−0.1%) and 20% (+−0.1%) risk were obtained by bootstrapping. Briefly, an age and sex model was estimated in a cohort of RA patients where 10 years of data was available (2006-2016) but where linked biomarker information was not because it preceded the availability of the MBDA test, which was first available in 2010. The cutpoints selected for categorization were low (0 to <1.3%), borderline risk (cumulative risk ≥1.3% to <1.8%), intermediate risk (≥1.8 to <5.2%), and high risk (≥5.2%).

For example, a VECTRA-CVD Score can be calculated according to Formula IX:

$$VECTRA - CVD Score = + 1.6 \; 076 \times tanh ( Adjusted MBDA / 33.0807 ) - 0.1711 \times ln ( Leptin ) + 0.5724 \times ln ( T NFR 1 ) + 0.1454 \times ln ( MMP 3 ) + 0.0314 \times age + 0.2691 \times smoking + 0.2732 \times diabetes + 0.2694 \times hypertension + 0.3378 \times history of CVD . \qquad Formula IX$$

Biostatistical tools were used to calculate a CVD 3-year Risk % for RA patients based on a VECTRA biomarker set and VECTRA-CVD Score obtained with clinical data.

Patients were grouped into three risk groups by their predicted risk for CVD at three years. The 3 year risk estimate % was calculated according to Formula X:

100×{1−A^{exp(B×VECTRA-CVD Score)}} Formula X

where A reflects the baseline risk, which is the probability of no CVD event within 3 years setting all covariates to zero, and B is the calibration constant applied to the test clinical data.

Testing and Internal Validation. The risk scores from training data for the five methods described above were compared in terms of their c index at three years (discrimination), calibration, and goodness of fit (observed versus expected at three years in decile categories of predicted risk), incremental contribution of MBDA-based CVD risk score method and Net Reclassification Index (NRI). The purpose of the NRI table was to evaluate whether a more complex method meaningfully changed how individual patients would have been classified in terms of risk category (low, intermediate, high etc.). In addition, survival curves were generated for each of the three predicted risk groups.

SAS 9.4 was used for data preparation, and R version 2.4 and R packages survival, nricens and pec were used for evaluating model performance, calculating NRIs and C-index, and generating plots.

Sensitivity analyses. A variety of sensitivity analyses were conducted to confirm that final method performance was consistent in key subgroups of patients. These included patients younger than 65, younger than 75, with and without diabetes, taking and not taking statins, with and without a history of CVD-related conditions. A sensitivity analysis was also conducted of patients that censored them when they added or switched to a biologic, to address the possibility that RA disease activity as measured by the biomarker might be meaningfully misclassified over time.

### Example 3: Advantageous Performance of VECTRA-CVD Score Determination for Rheumatoid Arthritis Patients

Cohort selection. After applying the inclusion and exclusion criteria, a total of 30,751 RA patients were eligible for the cohort. Baseline characteristics were: Mean age 69 years, 23% of patients were under age 65. 18% men. 8% were Black. The prevalence of various CVD-related comorbidities such as diabetes (40%) and hypertension (79%) was high, as was statin use (42%). Approximately 60% of patients were on methotrexate, 33% were on a TNF inhibitor (TNFi), and 15% were receiving non-TNFi biologics. The median CRP value was 1.5 mg/L, and the median MBDA score was 41, interpreted as being at the higher end of moderate RA disease activity. The median (IQR) amount of follow-up time after the index date was 1.7 (1.3, 2.7) years. The distribution of baseline candidate predictors in the RA cohort, including comorbidities using 12 month baseline and all available preceding data, is shown in Table 7.

TABLE 7
Distribution of baseline candidate predictors in RA cohort

| | Cohort | |
|---|---|---|
| Predictors | N = 30751 | |
| Age, mean(SD) | 68.8 | (9.6) |
| Age group, % | | |
| <65 | 23.3 | |
| 65-74 | 50.9 | |
| >74 | 25.8 | |
| Male, % | 18.2 | |
| Black Race, % | 8.4 | |
| Comorbidities | | |
| Diabetes, % | 39.8 | |
| Hypertension, % | 78.7 | |
| History of other CVD, % | 37.1 | |
| Past or current smoking, % | 24.5 | |
| Obesity | 12.1 | |
| Hyperlipidemia diagnosis | 54.4 | |
| Chronic kidney disease | 8.9 | |
| diagnosis | | |
| Medications | | |
| Statins | 42.4 | |
| Beta-blockers | 34.4 | |
| ACEI* | 25.9 | |
| ARB | 22.0 | |
| RA Medications | | |
| Methotrexate | 59.8 | |
| Other csDMARDs | 44.7 | |
| TNFi biologics | 32.8 | |
| Non-TNFi biologics | 14.8 | |
| Oral Glucocorticoids | 57.5 | |
| NSAIDS | 48.0 | |
| Biomarkers | | |
| C reactive protein, mg/L, | 1.5 | (1.5) |
| Mean (SD) | | |
| C reactive protein, mg/L, | 1.5 | (0.5-2.5) |
| Median (IQR) | | |
| Leptin^{†}, Mean (SD) | 1.5 | (0.5-2.5) |
| Leptin^{†}, Median (IQR) | 3.0 | (1.1) |
| MMP3^{†}, Mean (SD) | 3.2 | (2.3-3.9) |
| MMP3^{†}, Median (IQR) | 3.4 | (0.7) |
| TNFRI^{†}, Mean (SD) | 3.3 | (2.8-3.8) |
| TNFRI^{†}, Median (IQR) | 0.6 | (0.4) |
| Adjusted VECTRA score, | 40.9 | (13.5) |
| Mean (SD) | | |
| Adjusted VECTRA score, | 40.0 | (32.0-49.0) |
| Median (IQR) | | |

^{†}Natural log transformed
*ACEI = angiotension converting enzyme inhibitor; ARB = angiotensin receptor blocker

Training of the MBDA-based model. Age and sex parameters alone were significant, as were a variety of expected comorbidities including diabetes, hypertension, history of other CVD event, and smoking. However, in the final MBDA-based model, VECTRA-CVD Score, male sex and CRP were surprisingly no longer significant, and the final CVD risk score equation was 1.6076×tanh (VECTRA/33.0807)−0.1711× ln(Leptin)+0.5724×ln(TNFRI)+0.1454×ln(MMP3)+0.0314×age+0.2691×smoking+0.2732×diabetes+0.2694×hypertension+0.3378×history of CVD, where Leptin, TNFR1 and MMP3 represent the serum concentrations in ng/ml of the respective biomarkers.

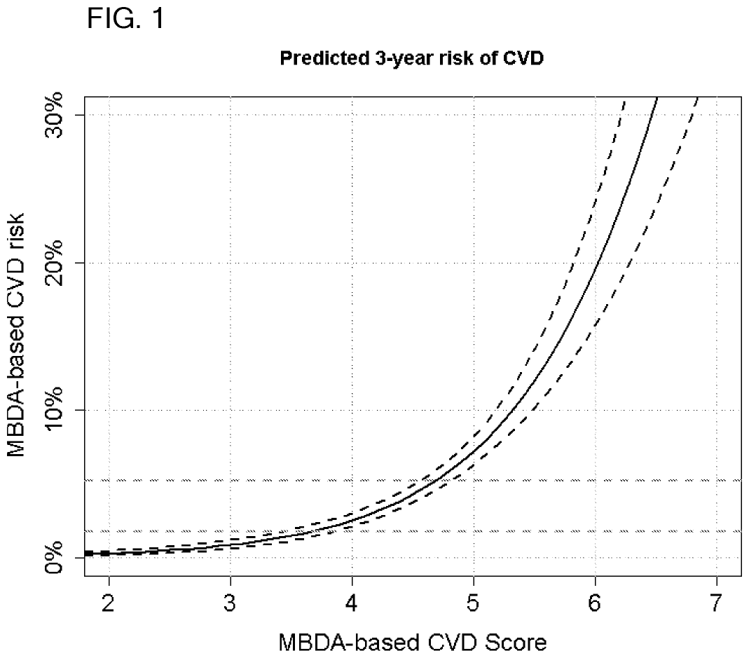

FIG. 1 shows a relationship between predictive VECTRA-CVD Score (x axis) and CVD 3-year Risk % (y axis) in an RA patient. In FIG. 1, the proportion of patients falling into low+borderline, intermediate, and high CVD 3-year Risk % categories was 15.1%, 54.3%, and 30.6%, respectively.

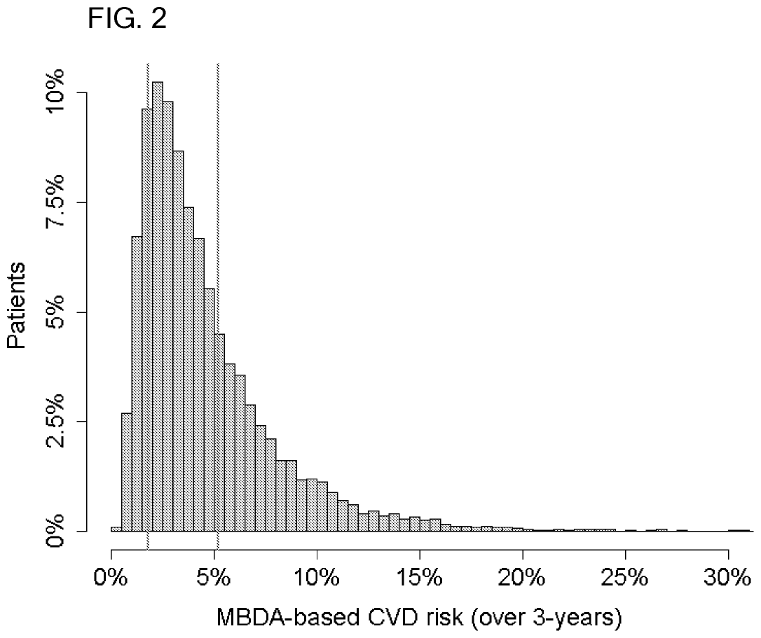

FIG. 2 shows a distribution of patients for the relationship in FIG. 1. The distribution in FIG. 2 shows that for every level of the predictive VECTRA-CVD Score, patients with a wide range of CVD 3-year Risk % were found. This distribution and relationship shows that the scores are predictively valid and meaningful for a patients over a wide range of risk levels.

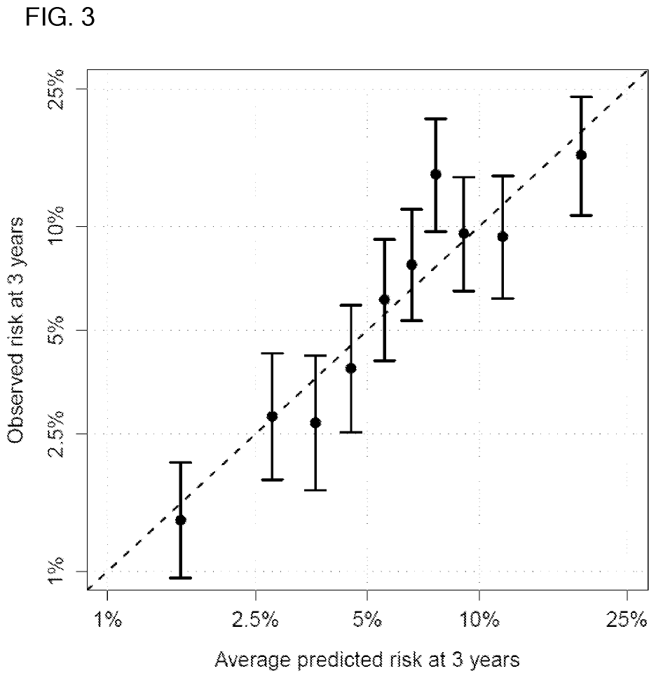

FIG. 3 shows a correlation between predictive CVD risk scoring (x axis) and CVD risk patient outcome (y axis). The goodness of fit of the final CVD 3-year Risk % across deciles in the internal validation dataset is shown in FIG. 3. As shown in FIG. 3, calibration of the final model was good across risk deciles, and the goodness of fit test statistic indicated reasonable fit (p=0.13). The actual observed risk was within the confidence bounds of predicted risk for almost all decile groups. Overall, the median (IQR) of predicted CVD 3-year Risk % was 3.4%. The goodness of fit of the data points to the dashed line indicates a very strong correlation exists between CVD 3-year Risk % and patient outcomes.

Comparison and evaluation in the internal validation dataset. The c-index (AUROC) for predicting CVD risk at three years increased across models and increased as covariates were added. It was 0.66 for the age+sex model, 0.67 with age, sex, and CRP, 0.69 for the parsimonious clinical model without CRP and, 0.70 for the parsimonious clinical model with CRP, and 0.72 for the final CVD 3-year Risk % method. In particular, predictions based on the final CVD 3-year Risk % method were surprisingly less variable and discriminated surprisingly better between patients than did a conventional method using age and sex only.

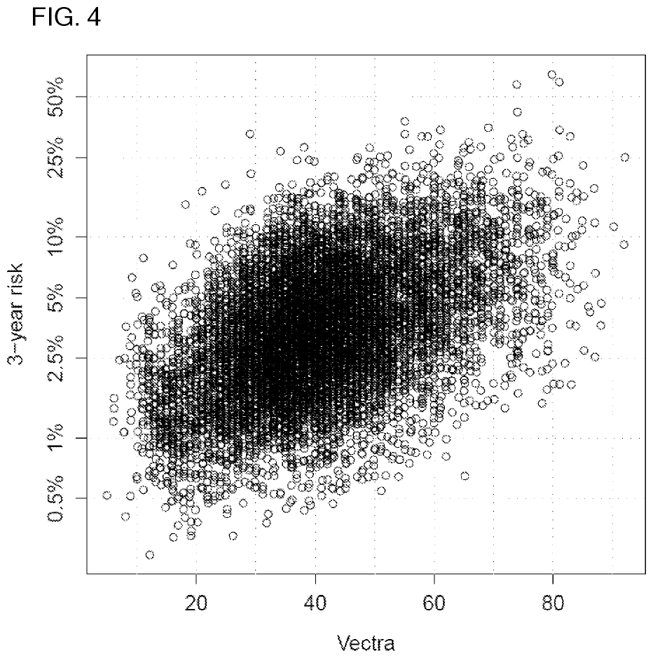

The CVD 3-year Risk % method advantageously accounted for the contribution that RA-related inflammation makes to CVD risk by including the VECTRA score as a co-variate. FIG. 4 illustrates the relationship between inflammation and CVD risk. In FIG. 4, results of the CVD 3-year Risk % method in the internal validation dataset is plotted according to the VECTRA score. A positive association was observed between increasing VECTRA score and predicted CVD risk. For every level of VECTRA score, a wide range of CVD risk values was observed, consistent with the varying contributions of the other covariates in the cohort.

Survival charts. CVD-event free survival plots were used to show the advantageous separation of the low, intermediate, and high risk group categories over time using the VECTRA-CVD Score and final CVD 3-year Risk % methods.

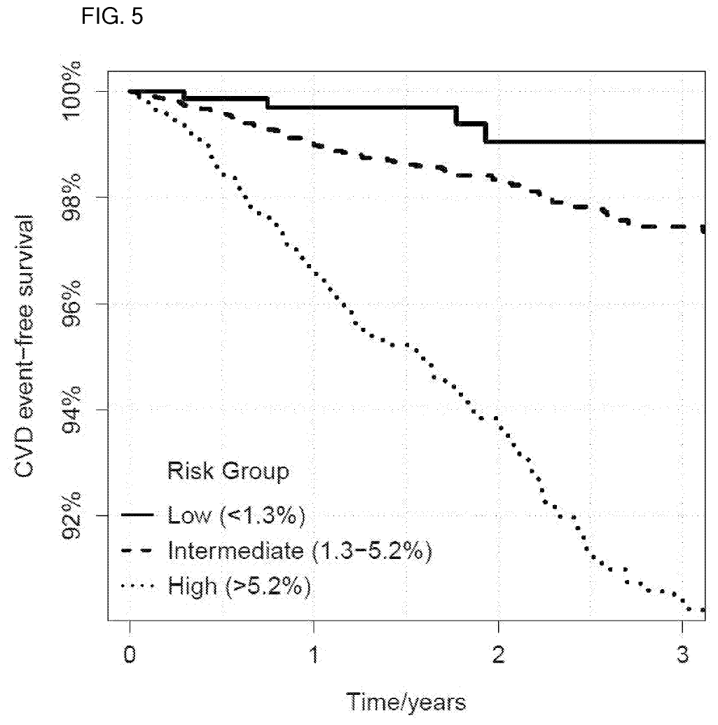

FIG. 5 shows results of a method for clinically validating the predictive ability of CVD 3-year Risk % values of this invention. FIG. 5 shows CVD-event-free survival rates for RA subjects. The plotted lines show survival rates corresponding to low, intermediate, and high CVD risk group thresholds for the predictive CVD 3-year Risk % values of this invention (n=10,275).

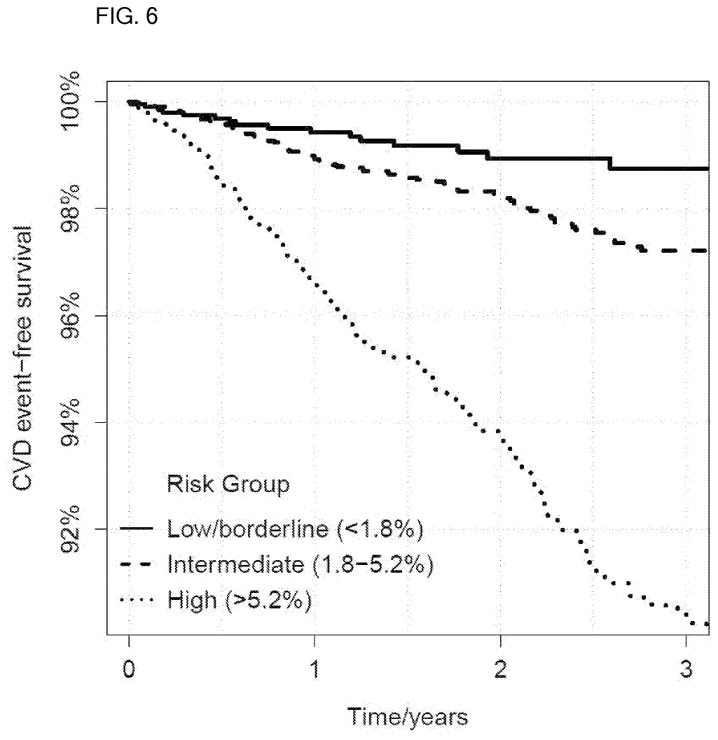

FIG. 6 shows results of a method for clinically validating the predictive ability of CVD 3-year Risk % values of this invention. FIG. 6 shows CVD-event-free survival rates for RA subjects. The plotted lines show survival rates corresponding to low/borderline, intermediate, and high CVD risk group thresholds for the predictive CVD 3-year Risk % values of this invention (n=10,275).

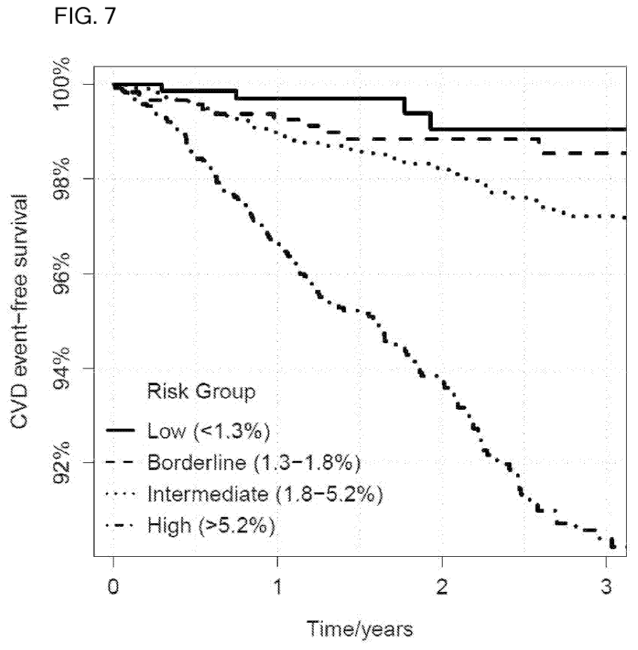

FIG. 7 shows results of a method for clinically validating the predictive ability of CVD 3-year Risk % values of this invention. FIG. 7 shows CVD-event-free survival rates for RA subjects. The plotted lines show survival rates corresponding to low, borderline, intermediate, and high CVD risk group thresholds for the predictive CVD 3-year Risk % values of this invention (n=10,275).

### Example 4: Comparison of Performance of VECTRA-CVD Score Determinations to Conventional Methods for Rheumatoid Arthritis Patients

Performance of VECTRA-CVD Score and CVD 3-year Risk % methods was compared to conventional methods for determining CVD risk in rheumatoid arthritis patients.

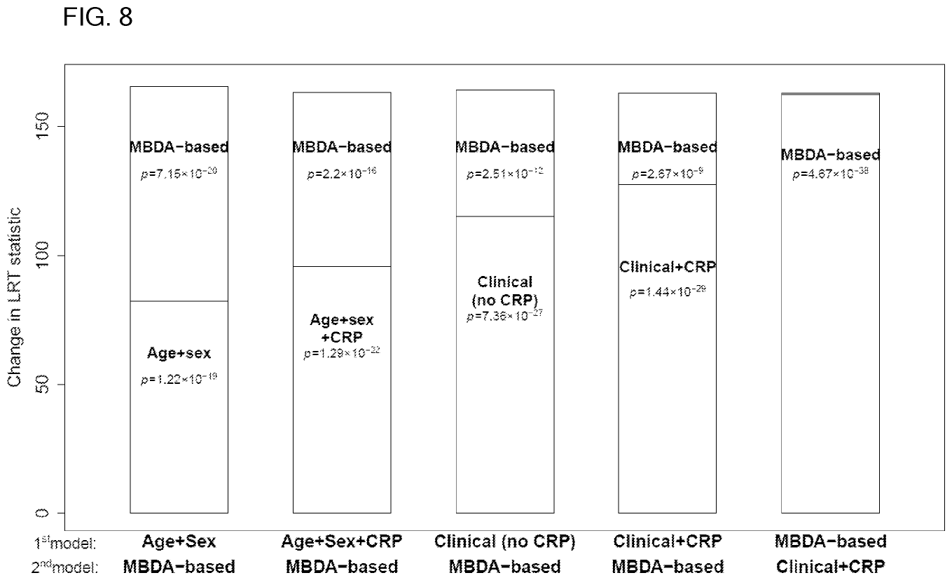

FIG. 8 shows the unexpectedly superior accuracy of a method of this invention for assessing CVD 3-year Risk % values for RA subjects. The bar chart in FIG. 8 shows that the method of this invention based on VECTRA-CVD Score (“MBDA-based”) was surprisingly far more accurate than various methods that did not include VECTRA-CVD Score. This may be seen by the incremental increases in the likelihood ratio test (LRT) (y axis, height of shaded portion) from left to right in FIG. 8.

In FIG. 8, the bar on the left shows that a determination of CVD risk based only on the clinical parameters age+sex (1^{st} model), when entered into the calculation first, made an incremental contribution to a VECTRA-CVD Score (2^{nd} model).

Likewise, the second, third and fourth bars from the left also show that determination of CVD risk based only on the parameters age+sex+CRP(biomarker), a set of clinical parameters without CRP, and a set of clinical parameters with CRP, respectively, also made incremental contributions to a VECTRA CVD Score (2^{nd} model) when entered into the calculation first. The set of clinical parameters was age, sex, diabetes, hypertension, smoking, and history of CVD.

Importantly, in FIG. 8, the bar on the right shows that in the VECTRA-CVD Score method (1^{st} model), when entered into the calculation first, the incremental contribution of the other methods was negligible (2^{nd} model, small shaded portion of bar at top). This is statistical proof of the fact that the VECTRA-CVD Score method disclosed herein exhibited unexpectedly superior accuracy for assessing CVD 3-year Risk % values for RA subjects over conventional methods.

In sum, FIG. 8 shows by bivariate analysis, that the use of the VECTRA-CVD Score of this invention achieved surprisingly high levels of accuracy in determining CVD 3-year Risk % values for RA subjects over conventional methods.

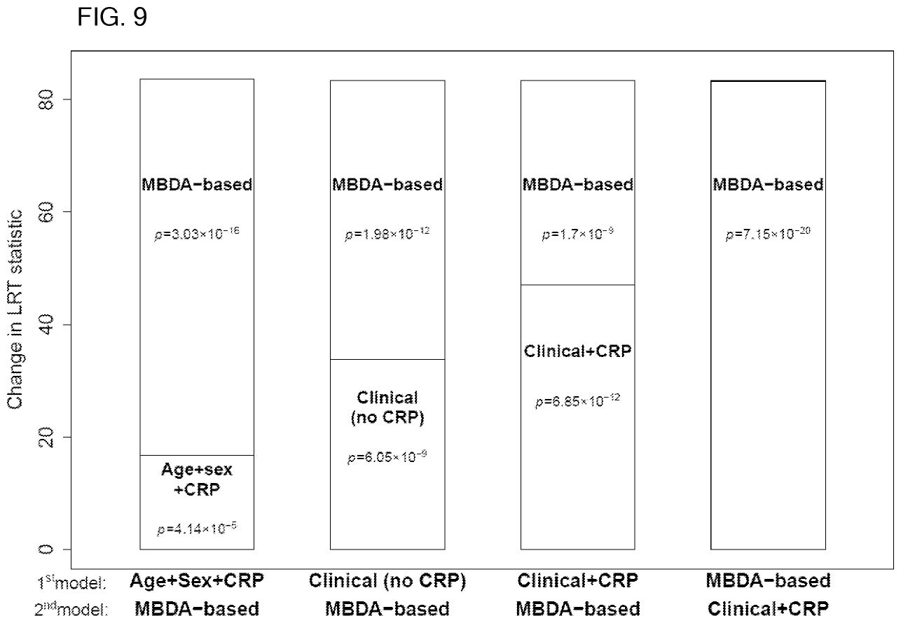

In a similar way, FIG. 9 shows the unexpectedly superior accuracy of a method of this invention for assessing CVD 3-year Risk % values for RA subjects. The bar chart in FIG. 9 shows that the method of this invention based on VECTRA-CVD Score (“MBDA-based”) was surprisingly far more accurate than various methods that did not include a VECTRA Score. This may be seen by the incremental increases in the likelihood ratio test (LRT) (y axis, height of shaded portion) from left to right in FIG. 9.

In FIG. 9, the bar on the left shows that a determination of CVD risk based only on the clinical parameters age+sex+CRP (1^{st} model), when entered into the calculation first, made an incremental contribution to a VECTRA-CVD Score (2^{nd} model).

Likewise, the second and third bars from the left also show that determination of CVD risk based only on a set of clinical parameters without CRP, and a set of clinical parameters with CRP, respectively (the P t models), also made incremental contributions to a VECTRA CVD Score (2^{nd} model) when entered into the calculation first. The set of clinical parameters was age, sex, diabetes, hypertension, smoking, and history of CVD.

Importantly, in FIG. 9, the bar on the right shows that in the VECTRA-CVD Score method (1^{st} model), when entered into the calculation first, the incremental contribution of the other methods was negligible (2^{nd} model, small shaded portion of bar at top). This is statistical proof of the fact that the VECTRA-CVD Score method disclosed herein exhibited unexpectedly superior accuracy for assessing CVD 3-year Risk % values for RA subjects over conventional methods.

In sum, FIG. 9 shows by bivariate analysis, that the use of the VECTRA-CVD Score of this invention achieved surprisingly high levels of accuracy in determining CVD 3-year Risk % values for RA subjects over conventional methods.

In addition, results from sensitivity analyses showed that findings were consistent in all key subgroups of interest. These included patients younger than 65, younger than 75, patients with and without diabetes, patients taking and not taking statins, patients with and w/o history of CVD, and regardless if patients added or switch biologics.

### Example 5: Comparison of Performance of VECTRA-CVD Score and CVD 3-Year Risk % Determinations to Conventional Methods for Rheumatoid Arthritis Patients Through Bivariate Analysis

A bivariate analysis of the performance of the final VECTRA-CVD Score method as compared to conventional methods for determining CVD risk in rheumatoid arthritis patients is shown in Table 8.

TABLE 8
Bivariate analysis of performance of final VECTRA-CVD Score method

| | | | | Bivariate with VECTRA-CVD Score | | | | |
|---|---|---|---|---|---|---|---|---|
| | | | | Conventional | | | | |
| | Univariate | | | non-VECTRA score | | VECTRA-CVD Score | | |
| | Hazard | | | Hazard | | Hazard | | |
| | Ratio | | | Ratio | | Ratio | | Increment |
| Method | (95% CI) | P-value | LRT | (95% CI) | P-value | (95% CI) | P-value | in LRT |
| VECTRA | 2.89 | 4.67 × 10⁻³⁸ | 162 | — | — | — | — | — |
| CVD | (2.46, 3.42) | | | | | | | |
| Age + | 3.44 | 1.22 × 10⁻¹⁹ | 82 | 1.34 | 0.084 | 2.62 | 7.15 × 10⁻²⁰ | 83 |
| Sex | (2.62, 4.53) | | | (0.96, 1.86) | | (2.14, 3.20) | | |
| Age + | 2.97 | 1.29 × 10⁻²² | 96 | 1.13 | 0.412 | 2.71 | 2.20 × 10⁻¹⁶ | 67 |
| Sex + | (2.38, 3.70) | | | (0.84, 1.54) | | (2.15, 3.41) | | |
| CRP | | | | | | | | |
| All | 3.08 | 7.34 × 10⁻²⁷ | 115 | 1.24 | 0.197 | 2.56 | 2.51 × 10⁻¹² | 49 |
| clinical | (2.50, 3.80) | | | (0.89, 1.71) | | (1.98, 3.29) | | |
| (no CRP) | | | | | | | | |
| All | 2.94 | 1.44 × 10⁻²⁹ | 128 | 1.12 | 0.526 | 2.67 | 2.67 × 10⁻⁹ | 35 |
| clinical | (2.43, 3.55) | | | (0.79, 1.60) | | (1.96, 3.63) | | |
| (+CRP) | | | | | | | | |

The methods of this invention were surprisingly accurate for assessing CVD 3-year Risk % using VECTRA-CVD Score values for RA subjects as compared to conventional non-VECTRA methods. Because of the uncertainty and unpredictability

Table 8 shows the unexpectedly superior accuracy of a method of this invention for assessing CVD 3-year Risk % using VECTRA-CVD Score values for RA subjects as compared to conventional non-VECTRA methods.

Table 8 shows by bivariate analysis that the method of this invention based on VECTRA-CVD Score (“MBDA-based”) was surprisingly far more accurate than various methods that did not include VECTRA-CVD Score.

For example, Table 8 shows by bivariate analysis that the p-values for the conventional non-VECTRA methods were quite large, from 0.084 to 0.526. This means that the conventional non-VECTRA methods, and their corresponding sets of variables, essentially add almost nothing to the result obtained by the VECTRA-CVD Score. The p-values for the VECTRA-CVD Scores are far lower, about 10⁻⁹ to 10⁻²⁰, representing far greater accuracy for methods using the VECTRA-CVD Scores and corresponding variables such as the VECTRA biomarkers.

In sum, Table 8 proves by bivariate analysis that the conventional sets of variables (age+sex, age+sex+CRP, etc.) are not responsible for the unexpectedly superior accuracy of the VECTRA-CVD Scores.

In sum, Table 8 shows by bivariate analysis, that the use of the VECTRA-CVD Score of this invention achieved surprisingly high levels of accuracy in determining CVD 3-year Risk % values for RA subjects over conventional methods.

Example 6: Comparison of performance of VECTRA-CVD Score and CVD 3-year Risk % determinations to conventional methods for rheumatoid arthritis patients through reclassification analysis. Validation of categories for CVD risk in RA patients using a multi-biomarker disease activity and clinical variables.

A net reclassification table can show that the methods of this invention expand the population of patients encompassed for treatment because they accurately assess risk of CVD in RA.

In Tables 9-20 below, the large number of patients reclassified shows that the population of patients encompassed for treatment can be expanded by methods of this invention which accurately assess risk of CVD in RA.

In Tables 9-20 below, the large number of patients reclassified shows that the kind of treatments for CVD or RA applied to a population of patients can be modified due to the reclassification of patient risk in methods of this invention which accurately assess risk of CVD in RA.

In Tables 9-20 below, the “observed” % values are the observed cumulative incidence of CVD events.

In Tables 9-20 below, more than one-third, and as high as 75% of patients were reclassified by the final CVD 3-year Risk % method of this invention. This data shows the surprisingly increased accuracy of the CVD 3-year Risk % method of this invention. The CVD 3-year Risk % method of this invention advantageously reclassified a substantial proportion of patients into a higher category of predicted CVD risk, which can provide improved therapy, prognosis, and monitoring of CVD-RA patients.

Table 9 shows that compared to the age+sex only method, the final CVD 3-year Risk % method of this invention reclassified a substantial proportion of patients into a higher category of predicted CVD risk.

TABLE 9
CVD 3-year Risk % of this invention and reclassification of
patients based on CVD risk in RA compared to Age + Sex method

| | | | | | Patients |
|---|---|---|---|---|---|
| | CVD 3-year Risk % | | | | Reclassified |
| | Low/Borderline | Intermediate | High | | by CVD 3- |
| CVD risk predicted | Risk | Risk | Risk | Total | year Risk % |
| by Age + Sex | (<1.8%) | (1.8%-<5.2%) | (≥5.2%) | patients N | N (%) |

Low/Borderline Risk

| | | | | | |
|---|---|---|---|---|---|
| Patients | 686 | 352 | 22 | 1060 | 374 (35.3) |
| Observed | 1.2% | 1.3% | 7.8% | — | — |

Intermediate Risk

| | | | | | |
|---|---|---|---|---|---|
| Patients | 1251 | 3807 | 1127 | 6185 | 2378 (38.4) |
| Observed | 1.4% | 2.9% | 9.0% | — | — |

High Risk

| | | | | | |
|---|---|---|---|---|---|
| Patients | 75 | 1209 | 1746 | 3030 | 1284 (42.4) |
| Observed | 2.7% | 4.3% | 10.5% | — | — |

Table 10 shows that compared to the age+sex+CRP method, the final CVD 3-year Risk % method of this invention reclassified a substantial proportion of patients into a different category of predicted CVD risk. Three categories were used: low/borderline, intermediate, and high.

TABLE 10
CVD 3-year Risk % of this invention and reclassification of patients based
on CVD risk in RA compared to use of Age + Sex + CRP method

| | | | | | Patients |
|---|---|---|---|---|---|
| | CVD 3-year Risk % | | | | Reclassified |
| | Low/Borderline | Intermediate | High | | by CVD 3- |
| CVD risk predicted | Risk | Risk | Risk | Total | year Risk % |
| by the Age + Sex + CRP | (<1.8%) | (1.8%-<5.2%) | (≥5.2%) | patients N | N (%) |

Low/Borderline Risk

| | | | | | |
|---|---|---|---|---|---|
| Patients | 713 | 303 | 14 | 1030 | 317 (30.8) |
| Observed | 1.6% | 2.1% | 10.2% | — | — |

Intermediate Risk

| | | | | | |
|---|---|---|---|---|---|
| Patients | 1278 | 4015 | 943 | 6236 | 2221 (35.6) |
| Observed | 1.1% | 2.8% | 7.1% | — | — |

High Risk

| | | | | | |
|---|---|---|---|---|---|
| Patients | 21 | 1050 | 1938 | 3009 | 1071 (35.6) |
| Observed | 9.3% | 4.4% | 11.4% | — | — |

Table 11 shows that compared to the Clinical Variables without CRP method, the final CVD 3-year Risk % method of this invention reclassified a substantial proportion of patients into a different category of predicted CVD risk. Three categories were used: low/borderline, intermediate, and high.

TABLE 11
CVD 3-year Risk % of this invention and reclassification of patients based
on CVD risk in RA compared to method using Clinical Variables without CRP

| | | | | | Patients |
|---|---|---|---|---|---|
| CVD risk predicted | CVD 3-year Risk % | | | | Reclassified |
| by the Clinical | Low/Borderline | Intermediate | High | | by CVD 3- |
| Variables* without | Risk | Risk | Risk | Total | year Risk % |
| CRP | (<1.8%) | (1.8%-<5.2%) | (≥5.2%) | patients N | N (%) |

Low/Borderline Risk

| | | | | | |
|---|---|---|---|---|---|
| Patients | 1022 | 314 | 6 | 1342 | 320 (23.8) |
| Observed | 1.1% | 1.8% | 0.0% | — | — |

Intermediate Risk

| | | | | | |
|---|---|---|---|---|---|
| Patients | 977 | 4047 | 697 | 5721 | 1674 (29.3) |
| Observed | 1.6% | 2.7% | 8.2% | — | — |

High Risk

| | | | | | |
|---|---|---|---|---|---|
| Patients | 13 | 1007 | 2192 | 3212 | 1020 (31.8) |
| Observed | 0.0% | 5.2% | 10.6% | — | — |

*Clinical Variables were age, sex, diabetes, hypertension, smoking, and history of CVD

Table 12 shows that compared to the Clinical Variables with CRP method, the final CVD 3-year Risk % method of this invention reclassified a substantial proportion of patients into a different category of predicted CVD risk. Three categories were used: low/borderline, intermediate, and high.

TABLE 12
CVD 3-year Risk % of this invention and reclassification of patients based
on CVD risk in RA compared to method using Clinical Variables with CRP

| | | | | | Patients |
|---|---|---|---|---|---|
| | CVD 3-year Risk % | | | | Reclassified |
| CVD risk predicted | Low/Borderline | Intermediate | High | | by CVD 3- |
| by the Clinical | Risk | Risk | Risk | Total | year Risk % |
| Variables* with CRP | (<1.8%) | (1.8%-<5.2%) | (≥5.2%) | patients N | N (%) |

Low/Borderline Risk

| | | | | | |
|---|---|---|---|---|---|
| Patients | 1188 | 287 | 5 | 1480 | 292 (19.7) |
| Observed | 1.2% | 1.6% | 0.0% | — | — |

Intermediate Risk

| | | | | | |
|---|---|---|---|---|---|
| Patients | 821 | 4265 | 558 | 5644 | 1379 (24.4) |
| Observed | 1.5% | 2.8% | 7.2% | — | — |

High Risk

| | | | | | |
|---|---|---|---|---|---|
| Patients | 3 | 816 | 2332 | 3151 | 819 (26.0) |
| Observed | 0.0% | 5.3% | 10.7% | — | — |

*Clinical Variables are age, sex, diabetes, hypertension, smoking, and history of CVD

Table 13 shows that compared to the Age+Sex method, the final CVD 3-year Risk % method of this invention reclassified a substantial proportion of patients into a different category of predicted CVD risk. Three categories were used: low, intermediate, and high.

TABLE 13
CVD 3-year Risk % of this invention and reclassification of patients
based on CVD risk in RA compared to use of Age + Sex method

| | | | | | Patients |
|---|---|---|---|---|---|
| | CVD 3-year Risk % | | | | Reclassified |
| | Low | Intermediate | High | | by CVD 3- |
| CVD risk predicted | Risk | Risk | Risk | Total | year Risk % |
| by Age + Sex | (<1.3%) | (1.3%-<5.2%) | (≥5.2%) | patients N | N (%) |

Low Risk

| | | | | | |
|---|---|---|---|---|---|
| Patients | 264 | 192 | 4 | 460 | 196 (42.6) |
| Observed | 0.6% | 2.5% | 0.0% | — | — |

Intermediate Risk

| | | | | | |
|---|---|---|---|---|---|
| Patients | 684 | 4956 | 1145 | 6785 | 1829 (27.0) |
| Observed | 1.0% | 2.5% | 9.1% | — | — |

High Risk

| | | | | | |
|---|---|---|---|---|---|
| Patients | 15 | 1269 | 1746 | 3030 | 1284 (42.4) |
| Observed | 0.0% | 4.2% | 10.5% | — | — |

Table 14 shows that compared to the Age+Sex+CRP method, the final CVD 3-year Risk % method of this invention reclassified a substantial proportion of patients into a different category of predicted CVD risk. Three categories were used: low, intermediate, and high.

TABLE 14
CVD 3-year Risk % of this invention and reclassification of patients based
on CVD risk in RA compared to use of Age + Sex + CRP method

| | | | | | Patients |
|---|---|---|---|---|---|
| | CVD 3-year Risk % | | | | Reclassified |
| | Low | Intermediate | High | | by CVD 3- |
| CVD risk predicted | Risk | Risk | Risk | Total | year Risk % |
| by Age + Sex + CRP | (<1.3%) | (1.3%-<5.2%) | (≥5.2%) | patients N | N (%) |

Low Risk

| | | | | | |
|---|---|---|---|---|---|
| Patients | 262 | 152 | 2 | 416 | 154 (37.0) |
| Observed | 0.7% | 5.2% | 81.8% | — | — |

Intermediate Risk

| | | | | | |
|---|---|---|---|---|---|
| Patients | 699 | 5196 | 955 | 6850 | 1654 (24.1) |
| Observed | 1.0% | 2.5% | 7.0% | — | — |

High Risk

| | | | | | |
|---|---|---|---|---|---|
| Patients | 2 | 1069 | 1938 | 3009 | 1071 (35.6) |
| Observed | 0.0% | 4.5% | 11.4% | — | — |

Table 15 shows that compared to the Clinical Variables without CRP method, the final CVD 3-year Risk % method of this invention reclassified a substantial proportion of patients into a different category of predicted CVD risk. Three categories were used: low, intermediate, and high.

TABLE 15
CVD 3-year Risk % of this invention and reclassification of patients based
on CVD risk in RA compared to method using Clinical Variables without CRP

| | | | | | Patients |
|---|---|---|---|---|---|
| CVD risk predicted | CVD 3-year Risk % | | | | Reclassified |
| by Clinical | Low | Intermediate | High | | by CVD 3- |
| Variables* without | Risk | Risk | Risk | Total | year Risk % |
| CRP | (<1.3%) | (1.3%-<5.2%) | (≥5.2%) | patients N | N (%) |

Low Risk

| | | | | | |
|---|---|---|---|---|---|
| Patients | 394 | 179 | 0 | 573 | 179 (31.2) |
| Observed | 0.4% | 1.6% | N/A | — | — |

Intermediate Risk

| | | | | | |
|---|---|---|---|---|---|
| Patients | 566 | 5221 | 703 | 6490 | 1269 (19.6) |
| Observed | 1.3% | 2.5% | 8.1% | — | — |

High Risk

| | | | | | |
|---|---|---|---|---|---|
| Patients | 3 | 1017 | 2192 | 3212 | 1020 (31.8) |
| Observed | 0.0% | 5.1% | 10.6% | — | — |

*Clinical Variables are age, sex, diabetes, hypertension, smoking, and history of CVD

Table 16 shows that compared to the Clinical Variables with CRP method, the final CVD 3-year Risk % method of this invention reclassified a substantial proportion of patients into a different category of predicted CVD risk. Three categories were used: low, intermediate, and high.

TABLE 16
CVD 3-year Risk % of this invention and reclassification of patients based
on CVD risk in RA compared to use of Clinical Variables with CRP

| | | | | | Patients |
|---|---|---|---|---|---|
| | CVD 3-year Risk % | | | | Reclassified |
| CVD risk predicted | Low | Intermediate | High | | by CVD 3- |
| by Clinical | Risk | Risk | Risk | Total | year Risk % |
| Variables* with CRP | (<1.3%) | (1.3%-<5.2%) | (≥5.2%) | patients N | N (%) |

Low Risk

| | | | | | |
|---|---|---|---|---|---|
| Patients | 450 | 177 | 0 | 627 | 177 (28.2) |
| Observed | 0.4% | 3.5% | N/A | — | — |

Intermediate Risk

| | | | | | |
|---|---|---|---|---|---|
| Patients | 513 | 5421 | 563 | 6497 | 1076 (16.6) |
| Observed | 1.4% | 2.5% | 7.1% | — | — |

High Risk

| | | | | | |
|---|---|---|---|---|---|
| Patients | 0 | 819 | 2332 | 3151 | 819 (26.0) |
| Observed | N/A | 5.2% | 10.7% | — | — |

*Clinical Variables are age, sex, diabetes, hypertension, smoking, and history of CVD

Table 17 shows that compared to the Age+Sex method, the final CVD 3-year Risk % method of this invention reclassified a substantial proportion of patients into a different category of predicted CVD risk. Four categories were used: low, borderline, intermediate, and high.

TABLE 17
CVD 3-year Risk % of this invention and reclassification of patients
based on CVD risk in RA compared to use of Age + Sex Variables

| | | | | | | Patients |
|---|---|---|---|---|---|---|
| | CVD 3-year Risk % | | | | | Reclassified |
| CVD risk | | Borderline | Intermediate | High | | by CVD 3- |
| predicted | Low Risk | Risk | Risk | Risk | Total | year Risk % |
| by Age + Sex | (<1.3%) | (1.3%-<1.8%) | (1.8%-<5.2%) | (≥5.2%) | patients N | N (%) |

Low Risk

| | | | | | | |
|---|---|---|---|---|---|---|
| Patients | 264 | 95 | 97 | 4 | 460 | 196 (42.6) |
| Observed | 0.6% | 5.4% | 0.0% | 0.0% | — | — |

Borderline Risk

| | | | | | | |
|---|---|---|---|---|---|---|
| Patients | 179 | 148 | 255 | 18 | 600 | 452 (75.3) |
| Observed | 0.0% | 1.0% | 1.8% | 10.7% | — | — |

Intermediate Risk

| | | | | | | |
|---|---|---|---|---|---|---|
| Patients | 505 | 746 | 3807 | 1127 | 6185 | 2378 (38.4) |
| Observed | 1.4% | 1.3% | 29% | 9.0% | — | — |

High Risk

| | | | | | | |
|---|---|---|---|---|---|---|
| Patients | 15 | 60 | 1209 | 1746 | 3030 | 1284 (42.4) |
| Observed | 0.0% | 3.3% | 4.3% | 10.5% | — | — |

Table 18 shows that compared to the Age+Sex+CRP method, the final CVD 3-year Risk % method of this invention reclassified a substantial proportion of patients into a different category of predicted CVD risk. Four categories were used: low, borderline, intermediate, and high.

TABLE 18
CVD 3-year Risk % of this invention and reclassification of patients based
on CVD risk in RA compared to use of Age + Sex + CRP method

| | | | | | | Patients |
|---|---|---|---|---|---|---|
| | CVD 3-year Risk % | | | | | Reclassified |
| CVD risk | | Borderline | Intermediate | High | | by CVD 3- |
| predicted by | Low Risk | Risk | Risk | Risk | Total | year Risk % |
| Age + Sex + CRP | (<1.3%) | (1.3%-<1.8%) | (1.8%-<5.2%) | (≥5.2%) | patients N | N (%) |

Low Risk

| | | | | | | |
|---|---|---|---|---|---|---|
| Patients | 262 | 87 | 65 | 2 | 416 | 154 (37.0) |
| Observed | 0.7% | 7.0% | 2.5% | 81.8% | — | — |

Borderline Risk

| | | | | | | |
|---|---|---|---|---|---|---|
| Patients | 215 | 149 | 238 | 12 | 614 | 465 (75.7) |
| Observed | 1.6% | 0.0% | 1.9% | 0.0% | — | — |

Intermediate Risk

| | | | | | | |
|---|---|---|---|---|---|---|
| Patients | 484 | 794 | 4015 | 943 | 6236 | 2221 (35.6) |
| Observed | 0.7% | 1.2% | 2.8% | 7.1% | — | — |

High Risk

| | | | | | | |
|---|---|---|---|---|---|---|
| Patients | 2 | 19 | 1050 | 1938 | 3009 | 1071 (35.6) |
| Observed | 0.0% | 9.7% | 4.4% | 11.4% | — | — |

Table 19 shows that compared to the Clinical Variables without CRP method, the final CVD 3-year Risk % method of this invention reclassified a substantial proportion of patients into a different category of predicted CVD risk. Four categories were used: low, borderline, intermediate, and high.

TABLE 19
CVD 3-year Risk % of this invention and reclassification of patients based
on CVD risk in RA compared to method using Clinical Variables without CRP

| CVD risk | | | | | | Patients |
|---|---|---|---|---|---|---|
| predicted | CVD 3-year Risk % | | | | | Reclassified |
| by Clinical | | Borderline | Intermediate | High | | by CVD 3- |
| Variables* | Low Risk | Risk | Risk | Risk | Total | year Risk % |
| without CRP | (<1.3%) | (1.3%-<1.8%) | (1.8%-<5.2%) | (≥5.2%) | patients N | N (%) |

Low Risk

| | | | | | | |
|---|---|---|---|---|---|---|
| Patients | 394 | 117 | 62 | 0 | 573 | 179 (31.2) |
| Observed | 0.4% | 1.2% | 2.2% | N/A | — | — |

Borderline Risk

| | | | | | | |
|---|---|---|---|---|---|---|
| Patients | 267 | 244 | 252 | 6 | 869 | 625 (71.9) |
| Observed | 0.0% | 3.1% | 1.7% | 0.0% | — | — |

Intermediate Risk

| | | | | | | |
|---|---|---|---|---|---|---|
| Patients | 299 | 678 | 4047 | 697 | 5721 | 1674 (29.3) |
| Observed | 2.5% | 1.2% | 2.7% | 8.2% | — | — |

High Risk

| | | | | | | |
|---|---|---|---|---|---|---|
| Patients | 3 | 10 | 1007 | 2192 | 3212 | 1020 (31.8) |
| Observed | 0.0% | 0.0% | 5.2% | 10.6% | — | — |

*Clinical Variables are age, sex, diabetes, hypertension, smoking, and history of CVD

Table 20 shows that compared to the Clinical Variables with CRP method, the final CVD 3-year Risk % method of this invention reclassified a substantial proportion of patients into a different category of predicted CVD risk. Four categories were used: low, borderline, intermediate, and high.

TABLE 20
CVD 3-year Risk % of this invention and Reclassification of patients based
on CVD risk in RA compared to use of Clinical Variables with CRP

| CVD risk | | | | | | Patients |
|---|---|---|---|---|---|---|
| predicted | CVD 3-year Risk % | | | | | Reclassified |
| by Clinical | | Borderline | Intermediate | High | | by CVD 3- |
| Variables* | Low Risk | Risk | Risk | Risk | Total | year Risk % |
| with CRP | (<1.3%) | (1.3%-<1.8%) | (1.8%-<5.2%) | (≥5.2%) | patients N | N (%) |

Low Risk

| | | | | | | |
|---|---|---|---|---|---|---|
| Patients | 450 | 125 | 52 | 0 | 327 | 177 (28.2) |
| Observed | 0.4% | 5.1% | 0.0% | N/A | — | — |

Borderline Risk

| | | | | | | |
|---|---|---|---|---|---|---|
| Patients | 309 | 304 | 235 | 5 | 853 | 549 (64.4) |
| Observed | 1.2% | 1.0% | 2.0% | 0.0% | — | — |

Intermediate Risk

| | | | | | | |
|---|---|---|---|---|---|---|
| Patients | 204 | 617 | 4265 | 558 | 5644 | 1379 (24.4) |
| Observed | 1.9% | 1.4% | 2.8% | 7.2% | — | — |

High Risk

| | | | | | | |
|---|---|---|---|---|---|---|
| Patients | 0 | 3 | 816 | 2332 | 3151 | 819 (26.0) |
| Observed | N/A | 0.0% | 5.3% | 10.7% | — | — |

*Clinical Variables are age, sex, diabetes, hypertension, smoking, and history of CVD

Example 7: Comparison of performance of VECTRA-CVD Score and CVD 3-year Risk % determinations to conventional methods for rheumatoid arthritis patients through hazard ratio analysis. Validation of categories for CVD risk in RA patients using a multi-biomarker disease activity and clinical variables.

Hazard ratios of various methods can show that the methods of this invention expand the population of patients encompassed for treatment because they accurately assess risk of CVD in RA.

In order to mitigate the elevated CVD risk in RA, appropriate risk stratification may be needed to identify high risk patients as targets for intervention. Methods of this invention can advantageously be made available to clinicians in real time, at the point of care where they are most actionable. For RA patients specifically, methods of this invention can take into account the influence of the disease and its features, including disease activity and systemic inflammation, to completely characterize the totality of CVD risk present in RA. Methods of this invention have therefore expanded populations for risk prediction to accurately determine CVD risk in RA patients.

An accurate method for predicting CVD event risk in RA patients that reflects the magnitude of RA disease activity and associated systemic inflammation can make CVD risk stratification more accurate and more accessible. Methods of this invention can therefore treat RA patients for CVD preventive care who would otherwise not have been treated by conventional methods.

Systemic inflammation has been assessed through one or more biomarkers that reflect RA disease activity. A multi-biomarker disease activity (MBDA) score can be used, which is highly correlated with RA disease activity as measured by the Disease Activity Score in 28 joints (DAS28). In both cross-sectional and longitudinal studies, the MBDA score tracked RA disease activity.

The biomarker-clinical methods of this invention can be implemented at the point of care for RA. Provided in this disclosure is an RA-specific CVD prediction score, the VECTRA-CVD Score, which uses RA-related biomarkers to predict CVD risk in conjunction with data for clinical variables. Clinical data can be obtained using clinical observations readily accessible to clinicians, or which can be extracted from electronic systems (e.g. electronic health records, or health plan claims data used for population health management). Methods of this invention can provide prognosis, as well as preventive CVD care in RA, and in particular include the component of RA-related systemic inflammation that contributes to excess CVD risk in RA.

Hazard ratios of various methods as compared to the methods of this invention are shown in Table 21. Hazard ratios of various methods for CVD risk were obtained with training data (n=20,476).

TABLE 21
Hazard ratio comparison analysis for CVD
3-year Risk % method of this invention

| | Method | | | | |
|---|---|---|---|---|---|
| | | | | | VECTRA |
| | | | | | CVD 3-year |
| | | Age + | Clinical | Clinical | Risk % |
| Variables | Age + sex | sex + CRP | with CRP | without CRP | method |
| Age, years | 1.05 (1.04- | 1.05 (1.04- | 1.04 (1.04- | 1.04 (1.03- | 1.03 (1.02- |
| | 1.06) | 1.06) | 1.05) | 1.05) | 1.04) |
| Male sex | 1.43 (1.19- | 1.43 (1.19- | 1.32 (1.09- | 1.31 (1.08- | — |
| | 1.73) | 1.73) | 1.60) | 1.58) | |
| Comorbidities | | | | | |
| Diabetes | — | — | 1.29 (1.10- | 1.31 (1.11- | 1.31 (1.11- |
| | | | 1.52) | 1.54) | 1.55) |
| Hypertension | — | — | 1.30 (1.00- | 1.35 (1.04- | 1.31 (1.01- |
| | | | 1.69) | 1.75) | 1.71) |
| History of | — | — | 1.44 (1.21- | 1.46 (1.24- | 1.40 (1.18- |
| other CVD | | | 1.71) | 1.74) | 1.66) |
| Smoking | — | — | 1.35 (1.12- | 1.42 (1.19- | 1.31 (1.09- |
| | | | 1.61) | 1.70) | 1.57) |
| Biomarkers | | | | | |
| CRP^{†} | — | 1.22 (1.16- | 1.20 (1.13- | — | — |
| | | 1.29) | 1.26) | | |
| Leptin^{†} | — | | — | — | 0.84 (0.79- |
| | | | | | 0.90) |
| MMP3^{†} | — | — | — | — | 1.16 (1.03- |
| | | | | | 1.30) |
| TNFRI^{†} | — | — | — | — | 1.77 (1.43- |
| | | | | | 2.19) |
| Adjusted | — | — | — | — | 4.99 (2.24- |
| VECTRA | | | | | 11.13) |
| score^{‡} | | | | | |

CRP: C-reactive protein; CVD: Cardiovascular disease; MMP3; TNFRI.
^{†}Serum concentrations (in ug/mL for CRP and ng/mL for Leptin, MMP3 and TNFR1), natural log transformed
^{‡}Hyperbolic tangent transformed (tanh(a*Adjusted VECTRA score: where a: 1/33.08073)

### Example 8: Comparison of Performance of VECTRA-CVD Score Methods

Reference information from TRIPOD. One procedure may be to apply an automated variable selection method in the multivariable modeling. Several variants are available in most current software, including forward selection, backward elimination, and their combination. Backward elimination starts with a full model comprising all potential predictors; variables are sequentially removed from the model until a prespecified stopping rule (such as a P value or the Akaike information criterion [AIC]) is satisfied. Forward selection starts with an empty model, and predictors are sequentially added until a prespecified stopping rule is satisfied.

Backward elimination can be generally preferred if automated predictor selection procedures are used because all correlations between predictors are considered in the modeling procedure. Use of automated predictor selection strategies during the multivariable modeling may yield overfitted and optimistic models, particularly when sample size is small. The extent of overfitting due to the use of predictor selection strategies may be estimated, however, and accounted for in so-called internal validation procedures.

The TRIPOD statement suggests consideration of automated predictor selection procedures (backward elimination may be preferred) or shrinkage methods like LASSO for multivariable model building and cautions to account for overfitting. Penalizing a coefficient of a known, strong predictor (like age in CVD), as lasso would do, can be undesirable. Additionally, subset selection procedures can provide better prediction accuracy than lasso in situations with high signal-to-noise (i.e. sparse features). It may be clinically pragmatic to have as parsimonious a model as possible. For these reasons we used backward elimination with a significance level of 0.05 as the inclusion criteria for variable selection.

One issue in these automated predictor selection procedures can be the criterion for predictors to be selected for inclusion in the model. Sometimes, the predictor's significance level (a) is set to 0.05, as is common for hypothesis testing. However, simulation studies indicate that a higher value should be considered, particularly in small data sets. In such cases, use of the AIC for selection can be an attractive option; it accounts for model fit while penalizing for the number of parameters being estimated and corresponds to using a=0.157.

### Example 9: Comparison of Methods for CVD Risk in RA Patients Using a Multi-Biomarker Disease Activity (MBDA) Test Coupled with Clinical Factors of VECTRA-CVD Score Methods

This example demonstrates multiple models for predicting CVD risk using alternate multi-biomarker disease activity scores (MBDA) coupled with clinical factors in patients with rheumatoid arthritis (RA).

The analysis was to develop and validate a method for cardiovascular disease risk among rheumatoid arthritis patients. Methods were also developed for a set of sub population of RA patients separately (e.g. stratified by age>=65 vs. <65). MBDA-linked Medicare data were used to develop and validate CVD risk prediction algorithms. The MBDA data were linked to Medicare through patients' birth date, sex, NPI and MBDA test date. The MBDA test date in Medicare were identified through specific and non-specific Current Procedural Terminology (CPT) codes. Patient eligibility criteria were described above in Example 1.

Primary Outcomes/dependent variables: The primary outcome was time to a composite CVD event that includes any of myocardial infarction (MI), stroke, and fatal CVD. Fatal CVD was identified through the claim-based algorithm described in Xie et al., PHARMACOEPIDEMIOLOGY AND DRUG SAFETY (2018) 27:740-750.

Predictors or stratifying variables (see below): Variables considered as potential predictors of CVD included: age, sex, 12 MBDA biomarkers and various MBDA scores, race, physician diagnosis for diabetes, hyperlipidemia, hypertension, obesity, smoking, chronic kidney disease; prescriptions for statin, other lipid lowering drug, anti-hypertensive drug (ARB, ACEI, CCB, Beta-blockers), glucocorticoid use, RA medications including MTX, other csDMARDs, NSAIDs, and biologics.

Statistical Considerations: Cox regression was used for model building. Lasso will be used for initial variable selection. Additional variable selection will be performed based on the variables hazard ratio, partial likelihood ratio test statistic and p-value, and practicality of inclusion on a commercial test request form.

See also, Curtis J R, Xie F, Chen L, et al., Biomarker-related risk for myocardial infarction and serious infections in patients with rheumatoid arthritis: a population-based study, Annals of the Rheumatic Diseases 2018; Vol. 77, pp. 386−392, which is hereby incorporated by reference in its entirety.

Definitions of CVD events: Composite CVD includes MI, stroke and fatal CVD. These are defined as follows: MI: One or more hospital diagnosis codes (410.x1) from inpatients hospital diagnosis codes in any position (primary or secondary position); also requires with at least one night stay in hospital unless the patient died; stroke: One or more hospital diagnosis codes for hemorrhagic or ischemic stroke (430.*, 431.*, 433.x1, 434.x1, 436.*, I60.xx, I61.xx, I63.xx,I67.89) from inpatient discharge (primary or secondary position); fatal CVD: fatal CVD will be identified using a claims-based algorithm with PPV>=0.80 (Xie et al., PHARMACOEPIDEMIOLOGY AND DRUG SAFETY (2018) 27:740-750).

Endpoint(s)/Outcome(s) Assessment: Time to composite CVD event was calculated as the difference in date of first eligible MBDA test to date of admission for MI or stroke, or to date of death for fatal CVD. For patients that did not experience the composite CVD event during follow-up, time to composite CVD event was censored at the difference in date of first eligible MBDA test to date of last follow-up.

Results: The following reports results for 16 alternative methods for determining CVD risk in RA patients of Example 1. The methods use different subsets of the data, where the variables in each model were chosen using backward elimination starting with MBDA (or adjusted MBDA), MBDA-squared (or adjusted MBDA-squared), the individual MBDA biomarkers, age, age-squared, and sex; and diagnosis of hypertension, hyperlipidemia, or diabetes.

In each case where sex is a variable, “M” means “male” and the reported hazard ration reflects CVD risk in males as compared to females. In some methods, patient having hypertension and/or on statin drugs were excluded from the analysis, but need not necessarily be excluded from use of the method.

In each of the methods, the components or variables of the method are the “parameters” listed in the corresponding table, the coefficient of each variable is called “parameter estimate”, and statistical significance (i.e., p-value) for each variable is called “Pr >ChiSq.”

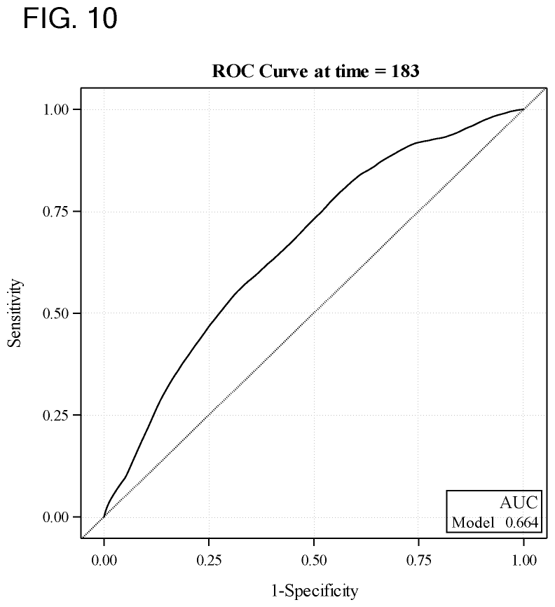

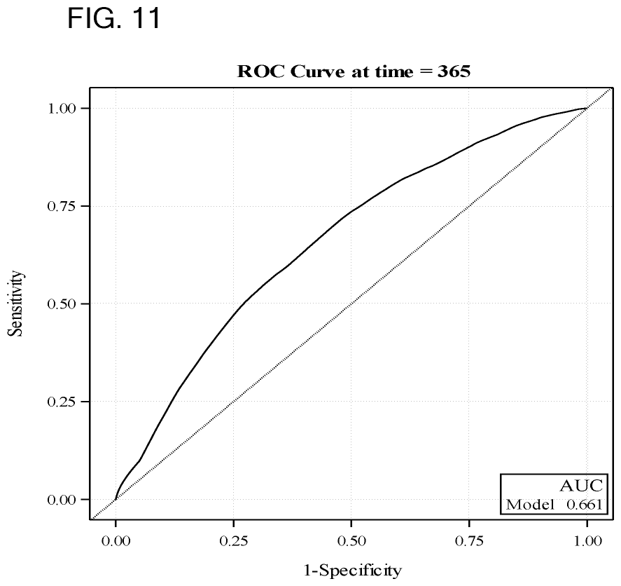

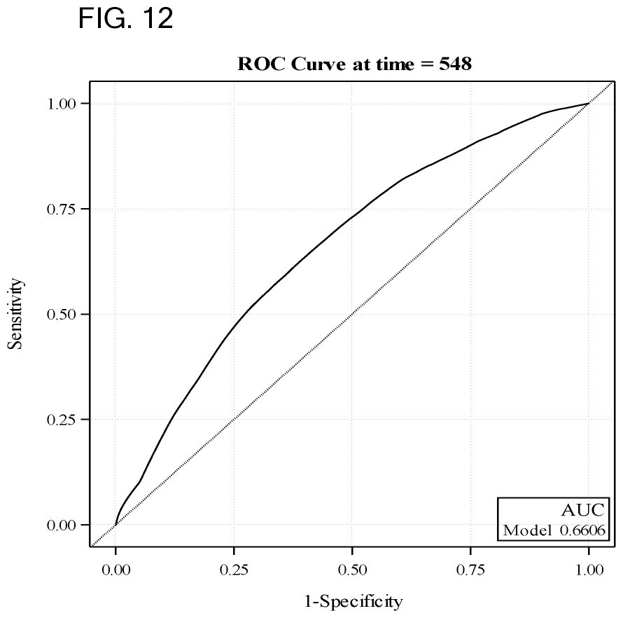

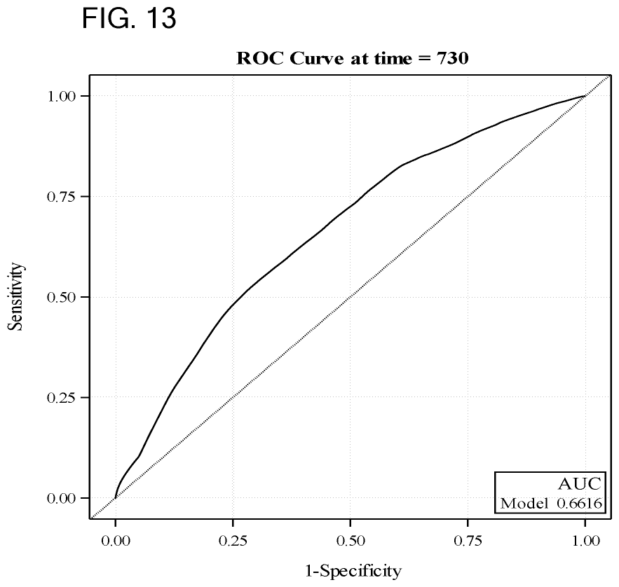

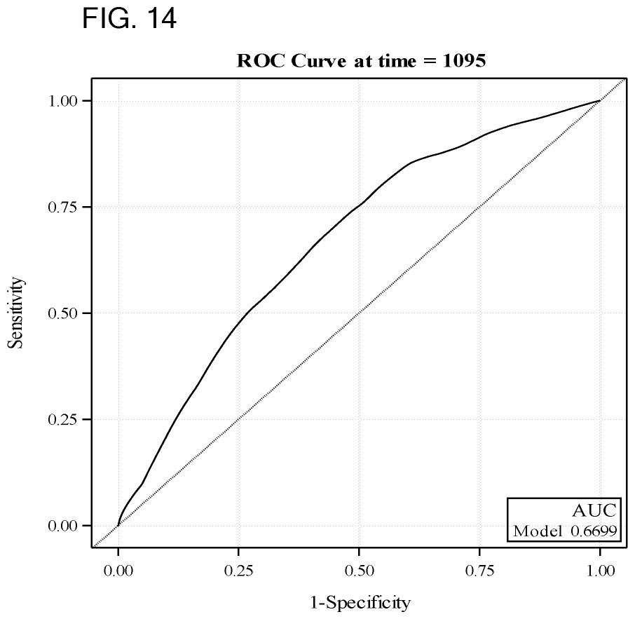

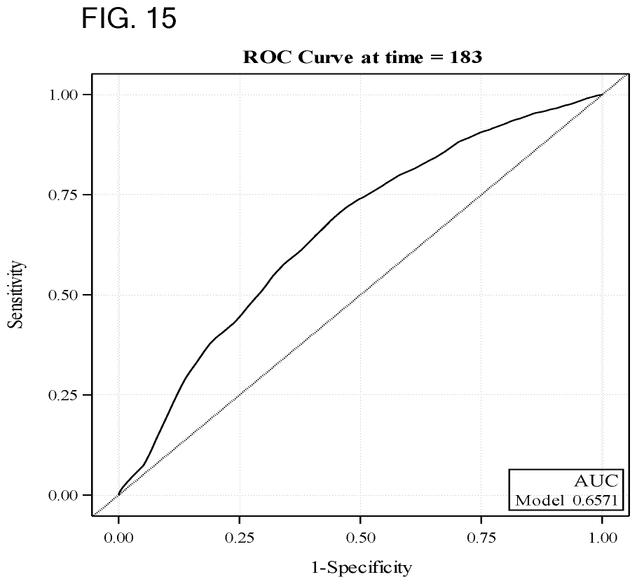

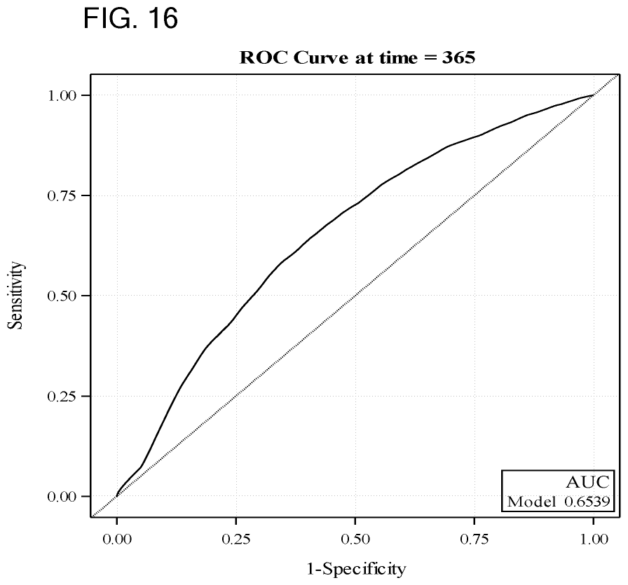

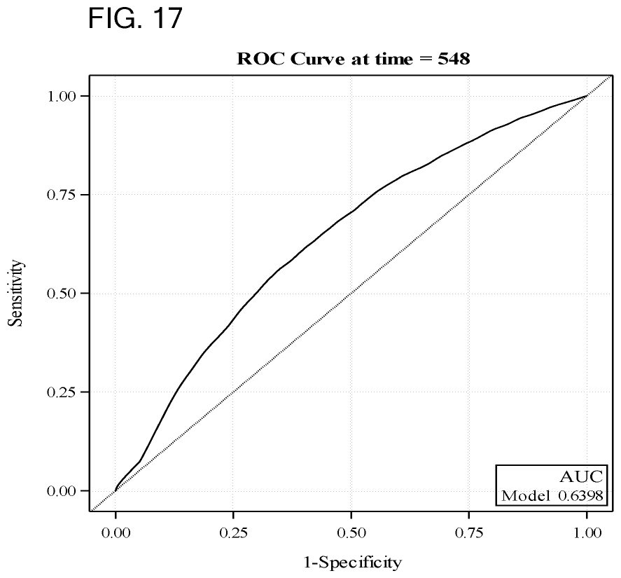

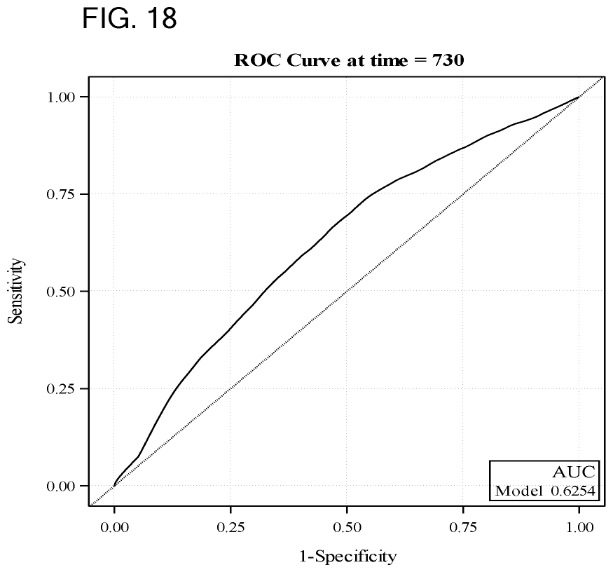

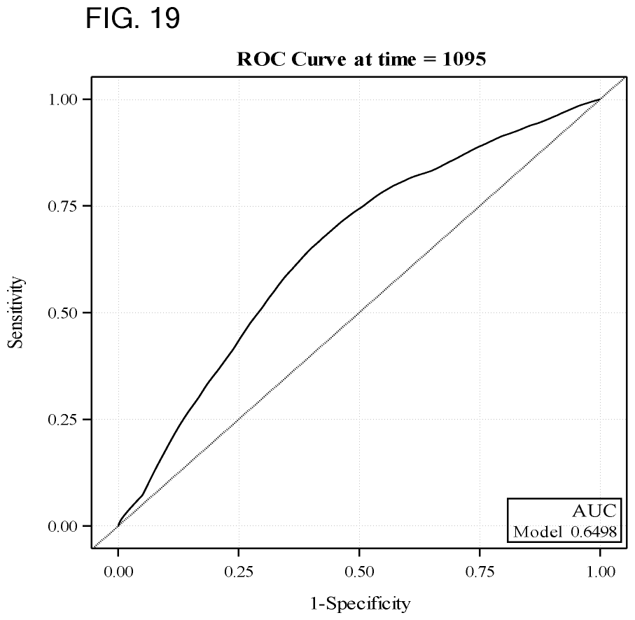

Example ROCs are shown in FIG. 10 through FIG. 19.

CVD risk method using MBDA and squared MBDA in patients aged 40 or older:

TABLE 22
CVD risk model
Type 3 Tests

| Effect | DF | Wald Chi-Square | Pr > ChiSq |
|---|---|---|---|
| Age | 1 | 48.1008 | <.0001 |
| MBDA_Score | 1 | 14.9407 | 0.0001 |
| Leptin | 1 | 40.2453 | <.0001 |
| MMP3 | 1 | 17.7250 | <.0001 |
| TNFR1 | 1 | 26.1810 | <.0001 |
| hypertension | 1 | 13.6353 | 0.0002 |
| diabetes | 1 | 13.5589 | 0.0002 |
| MBDASquare | 1 | 12.0338 | 0.0005 |

TABLE 23
Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.03175 | 0.00458 | 48.1008 | <.0001 | 1.032 | 1.023 | 1.042 |
| MBDA_Score | | 1 | 0.06328 | 0.01637 | 14.9407 | 0.0001 | 1.065 | 1.032 | 1.100 |
| Leptin | | 1 | −0.22220 | 0.03503 | 40.2453 | <.0001 | 0.801 | 0.748 | 0.858 |
| MMP3 | | 1 | 0.24258 | 0.05762 | 17.7250 | <.0001 | 1.275 | 1.138 | 1.427 |
| TNFR1 | | 1 | 0.53741 | 0.10503 | 26.1810 | <.0001 | 1.712 | 1.393 | 2.103 |
| hypertension | Y | 1 | 0.30070 | 0.08143 | 13.6353 | 0.0002 | 1.351 | 1.152 | 1.585 |
| diabetes | Y | 1 | 0.50298 | 0.13660 | 13.5589 | 0.0002 | 1.654 | 1.265 | 2.161 |
| MBDASquare | | 1 | −0.0005610 | 0.0001617 | 12.0338 | 0.0005 | 0.999 | 0.999 | 1.000 |

TABLE 24
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 19 | 0.0140 | 0.9058 |
| 2 | MMP1 | 1 | 18 | 0.0278 | 0.8675 |
| 3 | CCSC53 | 1 | 17 | 0.0424 | 0.8368 |
| 4 | IL6 | 1 | 16 | 0.3425 | 0.5584 |
| 5 | Resistin | 1 | 15 | 0.6118 | 0.4341 |
| 6 | SAA | 1 | 14 | 1.0233 | 0.3117 |
| 7 | SEX | 1 | 13 | 1.0321 | 0.3097 |
| 8 | ageSquare | 1 | 12 | 1.0193 | 0.3127 |
| 9 | EGF | 1 | 11 | 1.3954 | 0.2375 |
| 10 | VEGF | 1 | 10 | 2.7011 | 0.1003 |
| 11 | CRP | 1 | 9 | 3.0648 | 0.0800 |
| 12 | YKL40 | 1 | 8 | 2.6916 | 0.1009 |

CVD risk method using MBDA and squared MBDA in patients aged 40-75:

TABLE 25
CVD risk model
Type 3 Tests

| Effect | DF | Wald Chi-Square | Pr > ChiSq |
|---|---|---|---|
| Age | 1 | 10.9855 | 0.0009 |
| MBDA_Score | 1 | 8.5536 | 0.0034 |
| Leptin | 1 | 12.5151 | 0.0004 |
| MMP3 | 1 | 8.8042 | 0.0030 |
| TNFR1 | 1 | 7.1292 | 0.0076 |
| YKL40 | 1 | 9.0861 | 0.0026 |
| SEX | 1 | 3.8447 | 0.0499 |
| hypertension | 1 | 10.4327 | 0.0012 |
| diabetes | 1 | 8.0916 | 0.0044 |
| MBDASquare | 1 | 7.2742 | 0.0070 |

TABLE 26
Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02519 | 0.00760 | 10.9855 | 0.0009 | 1.026 | 1.010 | 1.041 |
| MBDA_Score | | 1 | 0.06000 | 0.02051 | 8.5536 | 0.0034 | 1.062 | 1.020 | 1.105 |
| Leptin | | 1 | −0.17664 | 0.04993 | 12.5151 | 0.0004 | 0.838 | 0.760 | 0.924 |
| MMP3 | | 1 | 0.21890 | 0.07377 | 8.8042 | 0.0030 | 1.245 | 1.077 | 1.438 |
| TNFR1 | | 1 | 0.38181 | 0.14300 | 7.1292 | 0.0076 | 1.465 | 1.107 | 1.939 |
| YKL40 | | 1 | 0.22187 | 0.07361 | 9.0861 | 0.0026 | 1.248 | 1.081 | 1.442 |
| SEX | M | 1 | 0.25018 | 0.12759 | 3.8447 | 0.0499 | 1.284 | 1.000 | 1.649 |
| hypertension | Y | 1 | 0.34082 | 0.10552 | 10.4327 | 0.0012 | 1.406 | 1.143 | 1.729 |
| diabetes | Y | 1 | 0.45629 | 0.16041 | 8.0916 | 0.0044 | 1.578 | 1.152 | 2.161 |
| MBDASquare | | 1 | −0.0005526 | 0.0002049 | 7.2742 | 0.0070 | 0.999 | 0.999 | 1.000 |

TABLE 27
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | CCSC53 | 1 | 19 | 0.0091 | 0.9241 |
| 2 | SAA | 1 | 18 | 0.0216 | 0.8831 |
| 3 | EGF | 1 | 17 | 0.0349 | 0.8519 |
| 4 | Resistin | 1 | 16 | 0.2095 | 0.6471 |
| 5 | VCAM1 | 1 | 15 | 0.3610 | 0.5479 |
| 6 | MMP1 | 1 | 14 | 0.6582 | 0.4172 |
| 7 | VEGF | 1 | 13 | 1.2411 | 0.2653 |
| 8 | IL6 | 1 | 12 | 1.7234 | 0.1893 |
| 9 | ageSquare | 1 | 11 | 1.7934 | 0.1805 |
| 10 | CRP | 1 | 10 | 2.9287 | 0.0870 |

CVD risk method using adjusted MBDA and squared adjusted MBDA in patients aged 40 and older:

TABLE 28
Type 3 Tests
Type 3 Tests

| Effect | DF | Wald Chi-Square | Pr > ChiSq |
|---|---|---|---|
| Age | 1 | 51.8135 | <.0001 |
| Adjusted_MBDA_Score | 1 | 15.4511 | <.0001 |
| Leptin | 1 | 33.9986 | <.0001 |
| MMP3 | 1 | 19.1995 | <.0001 |
| TNFR1 | 1 | 28.1287 | <.0001 |
| hypertension | 1 | 13.9294 | 0.0002 |
| diabetes | 1 | 13.5910 | 0.0002 |
| AdjMBDASquare | 1 | 12.7242 | 0.0004 |

TABLE 29
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.03369 | 0.00468 | 51.8135 | <.0001 | 1.034 | 1.025 | 1.044 |
| Adjusted_MBDA_Score | | 1 | 0.06133 | 0.01560 | 15.4511 | <.0001 | 1.063 | 1.031 | 1.096 |
| Leptin | | 1 | −0.20600 | 0.03533 | 33.9986 | <.0001 | 0.814 | 0.759 | 0.872 |
| MMP3 | | 1 | 0.25238 | 0.05760 | 19.1995 | <.0001 | 1.287 | 1.150 | 1.441 |
| TNFR1 | | 1 | 0.55323 | 0.10431 | 28.1287 | <.0001 | 1.739 | 1.417 | 2.133 |
| hypertension | Y | 1 | 0.30397 | 0.08144 | 13.9294 | 0.0002 | 1.355 | 1.155 | 1.590 |
| diabetes | Y | 1 | 0.50385 | 0.13667 | 13.5910 | 0.0002 | 1.655 | 1.266 | 2.163 |
| AdjMBDASquare | | 1 | −0.0005853 | 0.0001641 | 12.7242 | 0.0004 | 0.999 | 0.999 | 1.000 |

TABLE 30
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | CCSC53 | 1 | 19 | 0.0490 | 0.8249 |
| 2 | VCAM1 | 1 | 18 | 0.0539 | 0.8165 |
| 3 | MMP1 | 1 | 17 | 0.0525 | 0.8188 |
| 4 | SEX | 1 | 16 | 0.3658 | 0.5453 |
| 5 | IL6 | 1 | 15 | 0.6780 | 0.4103 |
| 6 | Resistin | 1 | 14 | 0.6345 | 0.4257 |
| 7 | ageSquare | 1 | 13 | 0.9913 | 0.3194 |
| 8 | SAA | 1 | 12 | 1.3845 | 0.2393 |
| 9 | EGF | 1 | 11 | 1.5700 | 0.2102 |
| 10 | VEGF | 1 | 10 | 2.8702 | 0.0902 |
| 11 | YKL40 | 1 | 9 | 3.5867 | 0.0582 |
| 12 | CRP | 1 | 8 | 3.1692 | 0.0750 |

CVD risk method using adjusted MBDA and squared adjusted MBDA in patients aged 40-75.

TABLE 31
Type 3 Tests
Type 3 Tests

| Effect | DF | Wald Chi-Square | Pr > ChiSq |
|---|---|---|---|
| Age | 1 | 10.9462 | 0.0009 |
| Adjusted_MBDA_Score | 1 | 7.0531 | 0.0079 |
| Leptin | 1 | 23.9201 | <.0001 |
| CRP | 1 | 4.9112 | 0.0267 |
| MMP3 | 1 | 14.7536 | 0.0001 |
| TNFR1 | 1 | 7.5575 | 0.0060 |
| YKL40 | 1 | 10.2049 | 0.0014 |
| hypertension | 1 | 11.4995 | 0.0007 |
| diabetes | 1 | 8.6174 | 0.0033 |
| AdjMBDASquare | 1 | 10.4380 | 0.0012 |

TABLE 32
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02547 | 0.00770 | 10.9462 | 0.0009 | 1.026 | 1.010 | 1.041 |
| Adjusted_MBDA_Score | | 1 | 0.05412 | 0.02038 | 7.0531 | 0.0079 | 1.056 | 1.014 | 1.099 |
| Leptin | | 1 | −0.22878 | 0.04678 | 23.9201 | <.0001 | 0.796 | 0.726 | 0.872 |
| CRP | | 1 | 0.14309 | 0.06457 | 4.9112 | 0.0267 | 1.154 | 1.017 | 1.310 |
| MMP3 | | 1 | 0.27686 | 0.07208 | 14.7536 | 0.0001 | 1.319 | 1.145 | 1.519 |
| TNFR1 | | 1 | 0.39263 | 0.14282 | 7.5575 | 0.0060 | 1.481 | 1.119 | 1.959 |
| YKL40 | | 1 | 0.23798 | 0.07450 | 10.2049 | 0.0014 | 1.269 | 1.096 | 1.468 |
| hypertension | Y | 1 | 0.35683 | 0.10523 | 11.4995 | 0.0007 | 1.429 | 1.163 | 1.756 |
| diabetes | Y | 1 | 0.47047 | 0.16027 | 8.6174 | 0.0033 | 1.601 | 1.169 | 2.192 |
| AdjMBDASquare | | 1 | −0.0006739 | 0.0002086 | 10.4380 | 0.0012 | 0.999 | 0.999 | 1.000 |

TABLE 33
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | CCSC53 | 1 | 19 | 0.0193 | 0.8895 |
| 2 | IL6 | 1 | 18 | 0.0193 | 0.8894 |
| 3 | MMP1 | 1 | 17 | 0.2521 | 0.6156 |
| 4 | Resistin | 1 | 16 | 0.2902 | 0.5901 |
| 5 | VCAM1 | 1 | 15 | 0.4731 | 0.4916 |
| 6 | SAA | 1 | 14 | 1.4195 | 0.2335 |
| 7 | EGF | 1 | 13 | 0.8085 | 0.3686 |
| 8 | VEGF | 1 | 12 | 1.3232 | 0.2500 |
| 9 | ageSquare | 1 | 11 | 1.8405 | 0.1749 |
| 10 | SEX | 1 | 10 | 2.7392 | 0.0979 |

CVD risk method using MBDA in patients aged 40 and older, with patients having hypertension excluded from the analysis:

TABLE 34
Type 3 Tests
Type 3 Tests

| Effect | DF | Wald Chi-Square | Pr > ChiSq |
|---|---|---|---|
| Age | 1 | 16.7624 | <.0001 |
| MBDA_Score | 1 | 8.4719 | 0.0036 |
| Leptin | 1 | 21.8046 | <.0001 |
| MMP3 | 1 | 9.0251 | 0.0027 |
| TNFR1 | 1 | 12.9110 | 0.0003 |
| YKL40 | 1 | 4.2823 | 0.0385 |
| diabetes | 1 | 5.4646 | 0.0194 |
| MBDASquare | 1 | 7.7424 | 0.0054 |

TABLE 35
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02686 | 0.00656 | 16.7624 | <.0001 | 1.027 | 1.014 | 1.041 |
| MBDA_Score | | 1 | 0.06490 | 0.02230 | 8.4719 | 0.0036 | 1.067 | 1.021 | 1.115 |
| Leptin | | 1 | −0.22631 | 0.04847 | 21.8046 | <.0001 | 0.797 | 0.725 | 0.877 |
| MMP3 | | 1 | 0.24419 | 0.08128 | 9.0251 | 0.0027 | 1.277 | 1.089 | 1.497 |
| TNFR1 | | 1 | 0.63635 | 0.17710 | 12.9110 | 0.0003 | 1.890 | 1.335 | 2.674 |
| YKL40 | | 1 | 0.17150 | 0.08288 | 4.2823 | 0.0385 | 1.187 | 1.009 | 1.396 |
| diabetes | Y | 1 | 0.35875 | 0.15346 | 5.4646 | 0.0194 | 1.432 | 1.060 | 1.934 |
| MBDASquare | | 1 | −0.0006126 | 0.0002201 | 7.7424 | 0.0054 | 0.999 | 0.999 | 1.000 |

TABLE 36
Summary of Backward Elimination

| | Effect | | Number | Wald | |
|---|---|---|---|---|---|
| Step | Removed | DF | In | Chi-Square | Pr > ChiSq |
| 1 | MMP1 | 1 | 18 | 0.0262 | 0.8714 |
| 2 | CCSC53 | 1 | 17 | 0.1360 | 0.7123 |
| 3 | Resistin | 1 | 16 | 0.2141 | 0.6436 |
| 4 | VCAM1 | 1 | 15 | 0.5880 | 0.4432 |
| 5 | SEX | 1 | 14 | 0.5876 | 0.4434 |
| 6 | ageSquare | 1 | 13 | 2.2459 | 0.1340 |
| 7 | VEGF | 1 | 12 | 3.1726 | 0.0749 |
| 8 | CRP | 1 | 11 | 2.2669 | 0.1322 |
| 9 | EGF | 1 | 10 | 1.0298 | 0.3102 |
| 10 | SAA | 1 | 9 | 0.4337 | 0.5102 |
| 11 | IL6 | 1 | 8 | 1.1335 | 0.2870 |

CVD risk method using MBDA in patients aged 40 and older, with patients taking statin drugs excluded from the analysis:

TABLE 37
Type 3 Tests
Type 3 Tests

| Effect | DF | Wald Chi-Square | Pr > ChiSq |
|---|---|---|---|
| Age | 1 | 29.2715 | <.0001 |
| MBDA_Score | 1 | 5.2882 | 0.0215 |
| Leptin | 1 | 9.7888 | 0.0018 |
| MMP3 | 1 | 5.3230 | 0.0210 |
| TNFR1 | 1 | 6.3332 | 0.0118 |
| SEX | 1 | 4.1607 | 0.0414 |
| hypertension | 1 | 9.7709 | 0.0018 |
| diabetes | 1 | 11.3308 | 0.0008 |

TABLE 38
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.03288 | 0.00608 | 29.2715 | <.0001 | 1.033 | 1.021 | 1.046 |
| MBDA_Score | | 1 | 0.01021 | 0.00444 | 5.2882 | 0.0215 | 1.010 | 1.002 | 1.019 |
| Leptin | | 1 | −0.15588 | 0.04982 | 9.7888 | 0.0018 | 0.856 | 0.776 | 0.943 |
| MMP3 | | 1 | 0.18187 | 0.07883 | 5.3230 | 0.0210 | 1.199 | 1.028 | 1.400 |
| TNFR1 | | 1 | 0.38937 | 0.15472 | 6.3332 | 0.0118 | 1.476 | 1.090 | 1.999 |
| SEX | M | 1 | 0.28740 | 0.14090 | 4.1607 | 0.0414 | 1.333 | 1.011 | 1.757 |
| hypertension | Y | 1 | 0.35240 | 0.11274 | 9.7709 | 0.0018 | 1.422 | 1.140 | 1.774 |
| diabetes | Y | 1 | 0.54655 | 0.16237 | 11.3308 | 0.0008 | 1.727 | 1.256 | 2.375 |

TABLE 39
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 19 | 0.0013 | 0.9709 |
| 2 | MMP1 | 1 | 18 | 0.0454 | 0.8312 |
| 3 | CCSC53 | 1 | 17 | 0.1403 | 0.7080 |
| 4 | Resistin | 1 | 16 | 0.7492 | 0.3867 |
| 5 | VEGF | 1 | 15 | 1.2215 | 0.2691 |
| 6 | EGF | 1 | 14 | 1.2258 | 0.2682 |
| 7 | YKL40 | 1 | 13 | 0.6254 | 0.4290 |
| 8 | SAA | 1 | 12 | 1.2016 | 0.2730 |
| 9 | IL6 | 1 | 11 | 1.3251 | 0.2497 |
| 10 | CRP | 1 | 10 | 0.9428 | 0.3316 |
| 11 | MBDASquare | 1 | 9 | 2.9171 | 0.0876 |
| 12 | ageSquare | 1 | 8 | 3.2219 | 0.0727 |

CVD risk method using MBDA in patients aged 40 and older, with patients with hypertension or taking statin drugs excluded from the analysis:

TABLE 40
Type 3 Tests
Type 3 Tests

| Effect | DF | Wald Chi-Square | Pr > ChiSq |
|---|---|---|---|
| Age | 1 | 11.4432 | 0.0007 |
| MBDA_Score | 1 | 0.7787 | 0.3775 |
| EGF | 1 | 8.1307 | 0.0044 |
| IL6 | 1 | 14.0333 | 0.0002 |
| CRP | 1 | 4.1400 | 0.0419 |
| SAA | 1 | 9.7176 | 0.0018 |
| TNFR1 | 1 | 7.1249 | 0.0076 |
| YKL40 | 1 | 9.1710 | 0.0025 |
| SEX | 1 | 8.4094 | 0.0037 |
| diabetes | 1 | 4.5943 | 0.0321 |
| MBDASquare | 1 | 7.2720 | 0.0070 |

TABLE 41
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02773 | 0.00820 | 11.4432 | 0.0007 | 1.028 | 1.012 | 1.045 |
| MBDA_Score | | 1 | −0.02663 | 0.03018 | 0.7787 | 0.3775 | 0.974 | 0.918 | 1.033 |
| EGF | | 1 | −0.41039 | 0.14392 | 8.1307 | 0.0044 | 0.663 | 0.500 | 0.880 |
| IL6 | | 1 | 0.73955 | 0.19742 | 14.0333 | 0.0002 | 2.095 | 1.423 | 3.085 |
| CRP | | 1 | 0.26476 | 0.13012 | 4.1400 | 0.0419 | 1.303 | 1.010 | 1.682 |
| SAA | | 1 | 0.48618 | 0.15596 | 9.7176 | 0.0018 | 1.626 | 1.198 | 2.207 |
| TNFR1 | | 1 | 0.60893 | 0.22813 | 7.1249 | 0.0076 | 1.838 | 1.176 | 2.875 |
| YKL40 | | 1 | 0.37139 | 0.12264 | 9.1710 | 0.0025 | 1.450 | 1.140 | 1.844 |
| SEX | M | 1 | 0.49189 | 0.16962 | 8.4094 | 0.0037 | 1.635 | 1.173 | 2.280 |
| diabetes | Y | 1 | 0.38006 | 0.17731 | 4.5943 | 0.0321 | 1.462 | 1.033 | 2.070 |
| MBDASquare | | 1 | −0.0008772 | 0.0003253 | 7.2720 | 0.0070 | 0.999 | 0.998 | 1.000 |

TABLE 42
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 18 | 0.0001 | 0.9943 |
| 2 | CCSC53 | 1 | 17 | 0.0194 | 0.8891 |
| 3 | Resistin | 1 | 16 | 0.0209 | 0.8851 |
| 4 | MMP1 | 1 | 15 | 0.1881 | 0.6645 |
| 5 | Leptin | 1 | 14 | 1.3744 | 0.2411 |
| 6 | VEGF | 1 | 13 | 2.9495 | 0.0859 |
| 7 | ageSquare | 1 | 12 | 3.4553 | 0.0630 |
| 8 | MMP3 | 1 | 11 | 3.7919 | 0.0515 |

CVD risk method using MBDA in patients aged 40-75, with patients with hypertension excluded from the analysis:

TABLE 43
Type 3 Tests
Type 3 Tests

| Effect | DF | Wald Chi-Square | Pr > ChiSq |
|---|---|---|---|
| Age | 1 | 1.9436 | 0.1633 |
| MBDA_Score | 1 | 4.0803 | 0.0434 |
| Leptin | 1 | 5.3915 | 0.0202 |
| MMP3 | 1 | 8.2865 | 0.0040 |
| TNFR1 | 1 | 6.7644 | 0.0093 |
| YKL40 | 1 | 11.3145 | 0.0008 |
| MBDASquare | 1 | 4.0849 | 0.0433 |

TABLE 44
Analysis of Maximum Likelihood Estimates
Analysis of Maximum Likelihood Estimates

| | | Parameter | Standard | Chi- | Pr > | Hazard | 95% Hazard Ratio | |
|---|---|---|---|---|---|---|---|---|
| Parameter | DF | Estimate | Error | Square | ChiSq | Ratio | Confidence Limits | |
| Age | 1 | 0.01446 | 0.01037 | 1.9436 | 0.1633 | 1.015 | 0.994 | 1.035 |
| MBDA_Score | 1 | 0.05585 | 0.02765 | 4.0803 | 0.0434 | 1.057 | 1.002 | 1.116 |
| Leptin | 1 | −0.14657 | 0.06312 | 5.3915 | 0.0202 | 0.864 | 0.763 | 0.977 |
| MMP3 | 1 | 0.29522 | 0.10256 | 8.2865 | 0.0040 | 1.343 | 1.099 | 1.643 |
| TNFR1 | 1 | 0.58425 | 0.22464 | 6.7644 | 0.0093 | 1.794 | 1.155 | 2.786 |
| YKL40 | 1 | 0.35250 | 0.10480 | 11.3145 | 0.0008 | 1.423 | 1.158 | 1.747 |
| MBDASquare | 1 | −0.0005597 | 0.000276 | 4.0849 | 0.0433 | 0.999 | 0.999 | 1.000 |

TABLE 45
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | CCSC53 | 1 | 18 | 0.1380 | 0.7103 |
| 2 | SAA | 1 | 17 | 0.2004 | 0.6544 |
| 3 | VEGF | 1 | 16 | 0.2714 | 0.6024 |
| 4 | EGF | 1 | 15 | 0.4324 | 0.5108 |
| 5 | IL6 | 1 | 14 | 0.2153 | 0.6426 |
| 6 | CRP | 1 | 13 | 0.2095 | 0.6472 |
| 7 | VCAM1 | 1 | 12 | 0.7947 | 0.3727 |
| 8 | Resistin | 1 | 11 | 1.5181 | 0.2179 |
| 9 | SEX | 1 | 10 | 1.6467 | 0.1994 |
| 10 | diabetes | 1 | 9 | 2.5951 | 0.1072 |
| 11 | MMP1 | 1 | 8 | 2.7378 | 0.0980 |
| 12 | ageSquare | 1 | 7 | 3.1408 | 0.0764 |

CVD risk method using MBDA in patients aged 40-75, with patients taking statin drugs excluded from the analysis:

TABLE 46
Type 3 Tests
Type 3 Tests

| Effect | DF | Wald Chi-Square | Pr > ChiSq |
|---|---|---|---|
| Age | 1 | 4.8301 | 0.0280 |
| MBDA_Score | 1 | 1.1529 | 0.2829 |
| MMP3 | 1 | 4.3032 | 0.0380 |
| YKL40 | 1 | 5.3459 | 0.0208 |
| SEX | 1 | 9.2204 | 0.0024 |
| hypertension | 1 | 6.4867 | 0.0109 |
| diabetes | 1 | 6.7264 | 0.0095 |

TABLE 47
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02074 | 0.00944 | 4.8301 | 0.0280 | 1.021 | 1.002 | 1.040 |
| MBDA_Score | | 1 | 0.00615 | 0.00573 | 1.1529 | 0.2829 | 1.006 | 0.995 | 1.018 |
| MMP3 | | 1 | 0.20353 | 0.09812 | 4.3032 | 0.0380 | 1.226 | 1.011 | 1.486 |
| YKL40 | | 1 | 0.21902 | 0.09473 | 5.3459 | 0.0208 | 1.245 | 1.034 | 1.499 |
| SEX | M | 1 | 0.49914 | 0.16438 | 9.2204 | 0.0024 | 1.647 | 1.194 | 2.273 |
| hypertension | Y | 1 | 0.36932 | 0.14501 | 6.4867 | 0.0109 | 1.447 | 1.089 | 1.922 |
| diabetes | Y | 1 | 0.48857 | 0.18838 | 6.7264 | 0.0095 | 1.630 | 1.127 | 2.358 |

TABLE 48
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | MMP1 | 1 | 19 | 0.0023 | 0.9614 |
| 2 | VCAM1 | 1 | 18 | 0.0118 | 0.9136 |
| 3 | CCSC53 | 1 | 17 | 0.0225 | 0.8808 |
| 4 | EGF | 1 | 16 | 0.0243 | 0.8761 |
| 5 | TNFR1 | 1 | 15 | 0.2142 | 0.6435 |
| 6 | VEGF | 1 | 14 | 0.3416 | 0.5589 |
| 7 | Resistin | 1 | 13 | 0.5285 | 0.4673 |
| 8 | SAA | 1 | 12 | 0.4750 | 0.4907 |
| 9 | CRP | 1 | 11 | 0.7073 | 0.4003 |
| 10 | Leptin | 1 | 10 | 1.2232 | 0.2687 |
| 11 | IL6 | 1 | 9 | 1.8597 | 0.1727 |
| 12 | MBDASquare | 1 | 8 | 1.5599 | 0.2117 |
| 13 | ageSquare | 1 | 7 | 2.9746 | 0.0846 |

CVD risk method using MBDA in patients aged 40-75, with patients with hypertension or taking statin drugs excluded from the analysis:

TABLE 49
Type 3 Tests
Type 3 Tests

| Effect | DF | Wald Chi-Square | Pr > ChiSq |
|---|---|---|---|
| Age | 1 | 1.2017 | 0.2730 |
| MBDA_Score | 1 | 0.3628 | 0.5470 |
| MMP3 | 1 | 4.1121 | 0.0426 |
| YKL40 | 1 | 10.8251 | 0.0010 |
| SEX | 1 | 7.6939 | 0.0055 |

TABLE 50
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.01294 | 0.01180 | 1.2017 | 0.2730 | 1.013 | 0.990 | 1.037 |
| MBDA_Score | | 1 | 0.00442 | 0.00733 | 0.3628 | 0.5470 | 1.004 | 0.990 | 1.019 |
| MMP3 | | 1 | 0.26101 | 0.12872 | 4.1121 | 0.0426 | 1.298 | 1.009 | 1.671 |
| YKL40 | | 1 | 0.40301 | 0.12249 | 10.8251 | 0.0010 | 1.496 | 1.177 | 1.902 |
| SEX | M | 1 | 0.58567 | 0.21114 | 7.6939 | 0.0055 | 1.796 | 1.187 | 2.717 |

TABLE 51
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | Resistin | 1 | 18 | 0.0025 | 0.9600 |
| 2 | VCAM1 | 1 | 17 | 0.0106 | 0.9182 |
| 3 | CCSC53 | 1 | 16 | 0.0155 | 0.9008 |
| 4 | Leptin | 1 | 15 | 0.1662 | 0.6835 |
| 5 | VEGF | 1 | 14 | 0.3104 | 0.5774 |
| 6 | SAA | 1 | 13 | 0.4471 | 0.5037 |
| 7 | EGF | 1 | 12 | 0.1540 | 0.6947 |
| 8 | CRP | 1 | 11 | 0.2805 | 0.5963 |
| 9 | TNFR1 | 1 | 10 | 0.3214 | 0.5708 |
| 10 | MBDASquare | 1 | 9 | 0.7789 | 0.3775 |
| 11 | IL6 | 1 | 8 | 1.2919 | 0.2557 |
| 12 | MMP1 | 1 | 7 | 1.4363 | 0.2307 |
| 13 | diabetes | 1 | 6 | 2.4742 | 0.1157 |
| 14 | ageSquare | 1 | 5 | 2.6038 | 0.1066 |

CVD risk method using adjusted MBDA in patients aged 40 and older, with patients having hypertension excluded from the analysis:

TABLE 52
Type 3 Tests
Type 3 Tests

| Effect | DF | Wald Chi-Square | Pr > ChiSq |
|---|---|---|---|
| Age | 1 | 17.4399 | <.0001 |
| Adjusted_MBDA_Score | 1 | 9.0152 | 0.0027 |
| Leptin | 1 | 19.9309 | <.0001 |
| MMP3 | 1 | 10.0875 | 0.0015 |
| TNFR1 | 1 | 13.5440 | 0.0002 |
| YKL40 | 1 | 4.4940 | 0.0340 |
| diabetes | 1 | 5.3643 | 0.0206 |
| AdjMBDASquare | 1 | 8.5851 | 0.0034 |

TABLE 53
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02808 | 0.00672 | 17.4399 | <.0001 | 1.028 | 1.015 | 1.042 |
| Adjusted_MBDA_Scor | | 1 | 0.06543 | 0.02179 | 9.0152 | 0.0027 | 1.068 | 1.023 | 1.114 |
| Leptin | | 1 | −0.21693 | 0.04859 | 19.9309 | <.0001 | 0.805 | 0.732 | 0.885 |
| MMP3 | | 1 | 0.25852 | 0.08139 | 10.0875 | 0.0015 | 1.295 | 1.104 | 1.519 |
| TNFR1 | | 1 | 0.64700 | 0.17581 | 13.5440 | 0.0002 | 1.910 | 1.353 | 2.695 |
| YKL40 | | 1 | 0.17569 | 0.08288 | 4.4940 | 0.0340 | 1.192 | 1.013 | 1.402 |
| diabetes | Y | 1 | 0.35579 | 0.15361 | 5.3643 | 0.0206 | 1.427 | 1.056 | 1.929 |
| AdjMBDASquare | | 1 | −0.0006669 | 0.0002276 | 8.5851 | 0.0034 | 0.999 | 0.999 | 1.000 |

TABLE 54
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | SEX | 1 | 18 | 0.0007 | 0.9783 |
| 2 | CCSC53 | 1 | 17 | 0.1520 | 0.6966 |
| 3 | MMP1 | 1 | 16 | 0.1734 | 0.6771 |
| 4 | Resistin | 1 | 15 | 0.2466 | 0.6195 |
| 5 | VCAM1 | 1 | 14 | 0.2830 | 0.5948 |
| 6 | ageSquare | 1 | 13 | 1.6852 | 0.1942 |
| 7 | VEGF | 1 | 12 | 2.9744 | 0.0846 |
| 8 | CRP | 1 | 11 | 2.7084 | 0.0998 |
| 9 | EGF | 1 | 10 | 1.9613 | 0.1614 |
| 10 | SAA | 1 | 9 | 1.3636 | 0.2429 |
| 11 | IL6 | 1 | 8 | 1.9303 | 0.1647 |

CVD risk method using adjusted MBDA in patients aged 40 and older, with patients taking statin drugs excluded from the analysis:

TABLE 55
Type 3 Tests
Type 3 Tests

| Effect | DF | Wald Chi-Square | Pr > ChiSq |
|---|---|---|---|
| Age | 1 | 27.5098 | <.0001 |
| Adjusted_MBDA_Scor | 1 | 5.9098 | 0.0151 |
| Leptin | 1 | 16.3204 | <.0001 |
| MMP3 | 1 | 10.1781 | 0.0014 |
| TNFR1 | 1 | 8.1445 | 0.0043 |
| hypertension | 1 | 10.7030 | 0.0011 |
| diabetes | 1 | 11.4330 | 0.0007 |
| AdjMBDASquare | 1 | 4.4790 | 0.0343 |

TABLE 56
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.03223 | 0.00615 | 27.5098 | <.0001 | 1.033 | 1.020 | 1.045 |
| Adjusted_MBDA_Scor | | 1 | 0.04968 | 0.02044 | 5.9098 | 0.0151 | 1.051 | 1.010 | 1.094 |
| Leptin | | 1 | −0.19374 | 0.04796 | 16.3204 | <.0001 | 0.824 | 0.750 | 0.905 |
| MMP3 | | 1 | 0.24813 | 0.07778 | 10.1781 | 0.0014 | 1.282 | 1.100 | 1.493 |
| TNFR1 | | 1 | 0.43318 | 0.15179 | 8.1445 | 0.0043 | 1.542 | 1.145 | 2.076 |
| hypertension | Y | 1 | 0.36798 | 0.11248 | 10.7030 | 0.0011 | 1.445 | 1.159 | 1.801 |
| diabetes | Y | 1 | 0.54920 | 0.16242 | 11.4330 | 0.0007 | 1.732 | 1.260 | 2.381 |
| AdjMBDASquare | | 1 | −0.0004474 | 0.0002114 | 4.4790 | 0.0343 | 1.000 | 0.999 | 1.000 |

TABLE 57
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 19 | 0.0147 | 0.9035 |
| 2 | MMP1 | 1 | 18 | 0.0230 | 0.8795 |
| 3 | CCSC53 | 1 | 17 | 0.1798 | 0.6716 |
| 4 | Resistin | 1 | 16 | 0.8219 | 0.3646 |
| 5 | VEGF | 1 | 15 | 1.2336 | 0.2667 |
| 6 | SEX | 1 | 14 | 1.5045 | 0.2200 |
| 7 | ageSquare | 1 | 13 | 2.0360 | 0.1536 |
| 8 | YKL40 | 1 | 12 | 2.6973 | 0.1005 |
| 9 | EGF | 1 | 11 | 1.9150 | 0.1664 |
| 10 | SAA | 1 | 10 | 2.5036 | 0.1136 |
| 11 | IL6 | 1 | 9 | 2.9234 | 0.0873 |
| 12 | CRP | 1 | 8 | 2.5900 | 0.1075 |

CVD risk method using adjusted MBDA in patients aged 40 and older, with patients with hypertension or taking statin drugs excluded from the analysis:

TABLE 58
Type 3 Tests
Type 3 Tests

| Effect | DF | Wald Chi-Square | Pr > ChiSq |
|---|---|---|---|
| Age | 1 | 0.0333 | 0.8553 |
| Adjusted_MBDA_Scor | 1 | 0.0036 | 0.9523 |
| EGF | 1 | 7.6136 | 0.0058 |
| IL6 | 1 | 13.2727 | 0.0003 |
| Leptin | 1 | 24.9718 | <.0001 |
| CRP | 1 | 4.4307 | 0.0353 |
| SAA | 1 | 8.5833 | 0.0034 |
| MMP3 | 1 | 5.4572 | 0.0195 |
| TNFR1 | 1 | 5.0851 | 0.0241 |
| YKL40 | 1 | 6.8320 | 0.0090 |
| diabetes | 1 | 5.2042 | 0.0225 |
| AdjMBDASquare | 1 | 10.8061 | 0.0010 |

TABLE 59
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.00188 | 0.01034 | 0.0333 | 0.8553 | 1.002 | 0.982 | 1.022 |
| Adjusted_MBDA_Scor | | 1 | −0.00182 | 0.03050 | 0.0036 | 0.9523 | 0.998 | 0.940 | 1.060 |
| EGF | | 1 | −0.32425 | 0.11751 | 7.6136 | 0.0058 | 0.723 | 0.574 | 0.910 |
| IL6 | | 1 | 0.59010 | 0.16197 | 13.2727 | 0.0003 | 1.804 | 1.313 | 2.478 |
| Leptin | | 1 | −0.35369 | 0.07078 | 24.9718 | <.0001 | 0.702 | 0.611 | 0.807 |
| CRP | | 1 | 0.22973 | 0.10914 | 4.4307 | 0.0353 | 1.258 | 1.016 | 1.558 |
| SAA | | 1 | 0.37034 | 0.12641 | 8.5833 | 0.0034 | 1.448 | 1.130 | 1.855 |
| MMP3 | | 1 | 0.24769 | 0.10603 | 5.4572 | 0.0195 | 1.281 | 1.041 | 1.577 |
| TNFR1 | | 1 | 0.50404 | 0.22352 | 5.0851 | 0.0241 | 1.655 | 1.068 | 2.565 |
| YKL40 | | 1 | 0.30487 | 0.11664 | 6.8320 | 0.0090 | 1.356 | 1.079 | 1.705 |
| diabetes | Y | 1 | 0.40863 | 0.17913 | 5.2042 | 0.0225 | 1.505 | 1.059 | 2.138 |
| AdjMBDASquare | | 1 | −0.0009768 | 0.0002972 | 10.8061 | 0.0010 | 0.999 | 0.998 | 1.000 |

TABLE 60
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | Resistin | 1 | 18 | 0.0189 | 0.8906 |
| 2 | CCSC53 | 1 | 17 | 0.0329 | 0.8560 |
| 3 | MMP1 | 1 | 16 | 0.0508 | 0.8217 |
| 4 | VCAM1 | 1 | 15 | 0.0790 | 0.7786 |
| 5 | SEX | 1 | 14 | 0.8738 | 0.3499 |
| 6 | ageSquare | 1 | 13 | 1.6678 | 0.1966 |
| 7 | VEGF | 1 | 12 | 1.9578 | 0.1617 |

CVD risk method using adjusted MBDA in patients aged 40-75, with patients with hypertension excluded from the analysis:

TABLE 61
Type 3 Tests
Type 3 Tests

| Effect | DF | Wald Chi-Square | Pr > ChiSq |
|---|---|---|---|
| Age | 1 | 2.0156 | 0.1557 |
| Adjusted_MBDA_Scor | 1 | 5.6442 | 0.0175 |
| Leptin | 1 | 5.5746 | 0.0182 |
| MMP3 | 1 | 9.4942 | 0.0021 |
| TNFR1 | 1 | 7.1172 | 0.0076 |
| YKL40 | 1 | 11.4511 | 0.0007 |
| AdjMBDASquare | 1 | 5.9581 | 0.0146 |

TABLE 62
Analysis of Maximum Likelihood Estimates
Analysis of Maximum Likelihood Estimates

| | | Parameter | Standard | Chi- | Pr > | Hazard | 95% Hazard Ratio | |
|---|---|---|---|---|---|---|---|---|
| Parameter | DF | Estimate | Error | Square | ChiSq | Ratio | Confidence Limits | |
| Age | 1 | 0.01494 | 0.01052 | 2.0156 | 0.1557 | 1.015 | 0.994 | 1.036 |
| Adjusted_MBDA_Scor | 1 | 0.06674 | 0.02809 | 5.6442 | 0.0175 | 1.069 | 1.012 | 1.130 |
| Leptin | 1 | −0.14862 | 0.06295 | 5.5746 | 0.0182 | 0.862 | 0.762 | 0.975 |
| MMP3 | 1 | 0.31601 | 0.10256 | 9.4942 | 0.0021 | 1.372 | 1.122 | 1.677 |
| TNFR1 | 1 | 0.59337 | 0.22242 | 7.1172 | 0.0076 | 1.810 | 1.171 | 2.799 |
| YKL40 | 1 | 0.35452 | 0.10476 | 11.4511 | 0.0007 | 1.425 | 1.161 | 1.750 |
| AdjMBDASquare | 1 | −0.0007074 | 0.0002898 | 5.9581 | 0.0146 | 0.999 | 0.999 | 1.000 |

TABLE 63
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | CCSC53 | 1 | 18 | 0.1765 | 0.6744 |
| 2 | SEX | 1 | 17 | 0.3847 | 0.5351 |
| 3 | VEGF | 1 | 16 | 0.6569 | 0.4176 |
| 4 | VCAM1 | 1 | 15 | 1.2235 | 0.2687 |
| 5 | SAA | 1 | 14 | 1.0150 | 0.3137 |
| 6 | EGF | 1 | 13 | 0.6859 | 0.4076 |
| 7 | IL6 | 1 | 12 | 0.6959 | 0.4042 |
| 8 | CRP | 1 | 11 | 0.7881 | 0.3747 |
| 9 | Resistin | 1 | 10 | 1.6377 | 0.2006 |
| 10 | MMP1 | 1 | 9 | 2.4122 | 0.1204 |
| 11 | diabetes | 1 | 8 | 2.5230 | 0.1122 |
| 12 | ageSquare | 1 | 7 | 3.0733 | 0.0796 |

CVD risk method using adjusted MBDA in patients aged 40-75, with patients taking statin drugs excluded from the analysis:

TABLE 64
Type 3 Tests
Type 3 Tests

| Effect | DF | Wald Chi-Square | Pr > ChiSq |
|---|---|---|---|
| Age | 1 | 5.3055 | 0.0213 |
| Adjusted_MBDA_Scor | 1 | 1.1360 | 0.2865 |
| MMP3 | 1 | 4.2557 | 0.0391 |
| YKL40 | 1 | 5.4684 | 0.0194 |
| SEX | 1 | 9.0424 | 0.0026 |
| hypertension | 1 | 6.6696 | 0.0098 |
| diabetes | 1 | 6.9929 | 0.0082 |

TABLE 65
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.02202 | 0.00956 | 5.3055 | 0.0213 | 1.022 | 1.003 | 1.042 |
| Adjusted_MBDA_Scor | | 1 | 0.00613 | 0.00575 | 1.1360 | 0.2865 | 1.006 | 0.995 | 1.018 |
| MMP3 | | 1 | 0.20301 | 0.09841 | 4.2557 | 0.0391 | 1.225 | 1.010 | 1.486 |
| YKL40 | | 1 | 0.22053 | 0.09431 | 5.4684 | 0.0194 | 1.247 | 1.036 | 1.500 |
| SEX | M | 1 | 0.49119 | 0.16335 | 9.0424 | 0.0026 | 1.634 | 1.187 | 2.251 |
| hypertension | Y | 1 | 0.37402 | 0.14482 | 6.6696 | 0.0098 | 1.454 | 1.094 | 1.931 |
| diabetes | Y | 1 | 0.49803 | 0.18833 | 6.9929 | 0.0082 | 1.645 | 1.138 | 2.380 |

TABLE 66
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 19 | 0.0183 | 0.8923 |
| 2 | MMP1 | 1 | 18 | 0.0265 | 0.8706 |
| 3 | CCSC53 | 1 | 17 | 0.0459 | 0.8304 |
| 4 | TNFR1 | 1 | 16 | 0.2359 | 0.6272 |
| 5 | EGF | 1 | 15 | 0.6814 | 0.4091 |
| 6 | VEGF | 1 | 14 | 0.4296 | 0.5122 |
| 7 | Resistin | 1 | 13 | 0.6878 | 0.4069 |
| 8 | SAA | 1 | 12 | 1.2852 | 0.2569 |
| 9 | CRP | 1 | 11 | 1.4923 | 0.2219 |
| 10 | Leptin | 1 | 10 | 1.5220 | 0.2173 |
| 11 | IL6 | 1 | 9 | 1.7241 | 0.1892 |
| 12 | AdjMBDASquare | 1 | 8 | 1.9257 | 0.1652 |
| 13 | ageSquare | 1 | 7 | 2.9136 | 0.0878 |

CVD risk method using adjusted MBDA in patients aged 40-75, with patients with hypertension or taking statin drugs excluded from the analysis:

TABLE 67
Type 3 Tests
Type 3 Tests

| Effect | DF | Wald Chi-Square | Pr > ChiSq |
|---|---|---|---|
| Age | 1 | 1.2445 | 0.2646 |
| Adjusted_MBDA_Scor | 1 | 0.0982 | 0.7540 |
| MMP3 | 1 | 4.5848 | 0.0323 |
| YKL40 | 1 | 11.8083 | 0.0006 |
| SEX | 1 | 7.4406 | 0.0064 |

TABLE 68
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.01336 | 0.01198 | 1.2445 | 0.2646 | 1.013 | 0.990 | 1.038 |
| Adjusted MBDA Score | | 1 | 0.00232 | 0.00739 | 0.0982 | 0.7540 | 1.002 | 0.988 | 1.017 |
| MMP3 | | 1 | 0.27715 | 0.12943 | 4.5848 | 0.0323 | 1.319 | 1.024 | 1.700 |
| YKL40 | | 1 | 0.41730 | 0.12144 | 11.8083 | 0.0006 | 1.518 | 1.196 | 1.926 |
| SEX | M | 1 | 0.57254 | 0.20990 | 7.4406 | 0.0064 | 1.773 | 1.175 | 2.675 |

TABLE 69
Summary of Backward Elimination
Summary of Backward Elimination

| | Effect | | Number | Wald | |
|---|---|---|---|---|---|
| Step | Removed | DF | In | Chi-Square | Pr > ChiSq |
| 1 | VCAM1 | 1 | 18 | 0.0001 | 0.9941 |
| 2 | Resistin | 1 | 17 | 0.0149 | 0.9027 |
| 3 | CCSC53 | 1 | 16 | 0.0312 | 0.8598 |
| 4 | TNFR1 | 1 | 15 | 0.1454 | 0.7030 |
| 5 | MMP1 | 1 | 14 | 0.4903 | 0.4838 |
| 6 | VEGF | 1 | 13 | 0.5058 | 0.4770 |
| 7 | SAA | 1 | 12 | 1.9714 | 0.1603 |
| 8 | Leptin | 1 | 11 | 0.5415 | 0.4618 |
| 9 | EGF | 1 | 10 | 0.3600 | 0.5485 |
| 10 | CRP | 1 | 9 | 0.4603 | 0.4975 |
| 11 | AdiMBDASquare | 1 | 8 | 1.8981 | 0.1683 |
| 12 | IL6 | 1 | 7 | 1.8220 | 0.1771 |
| 13 | ageSquare | 1 | 6 | 2.4670 | 0.1163 |
| 14 | diabetes | 1 | 5 | 2.6421 | 0.1041 |

CVD risk model in patient aged 40-75 including age, sex, and CRP:

TABLE 70
Type 3 Tests
Type 3 Tests

| Effect | DF | Wald Chi-Square | Pr > ChiSq |
|---|---|---|---|
| Age | 1 | 29.1978 | <.0001 |
| SEX | 1 | 20.5571 | <.0001 |
| CRP | 1 | 29.9101 | <.0001 |

TABLE 71
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.04073 | 0.00754 | 29.1978 | <.0001 | 1.042 | 1.026 | 1.057 |
| SEX | M | 1 | 0.51128 | 0.11277 | 20.5571 | <.0001 | 1.667 | 1.337 | 2.080 |
| CRP | | 1 | 0.18874 | 0.03451 | 29.9101 | <.0001 | 1.208 | 1.129 | 1.292 |

CVD risk method in patients aged 40 and older including age, sex, and CRP:

TABLE 72
Type 3 Tests
Type 3 Tests

| Effect | DF | Wald Chi-Square | Pr > ChiSq |
|---|---|---|---|
| Age | 1 | 129.7913 | <.0001 |
| SEX | 1 | 17.2474 | <.0001 |
| CRP | 1 | 42.1629 | <.0001 |

TABLE 73
Analysis of Maximum Likelihood Estimates
Analysis of Maximum Likelihood Estimates

| | | | Parameter | Standard | Chi- | Pr > | Hazard | 95% Hazard Ratio | |
|---|---|---|---|---|---|---|---|---|---|
| Parameter | | DF | Estimate | Error | Square | ChiSq | Ratio | Confidence Limits | |
| Age | | 1 | 0.05092 | 0.00447 | 129.7913 | <.0001 | 1.052 | 1.043 | 1.062 |
| SEX | M | 1 | 0.38532 | 0.09278 | 17.2474 | <.0001 | 1.470 | 1.226 | 1.763 |
| CRP | | 1 | 0.17261 | 0.02658 | 42.1629 | <.0001 | 1.188 | 1.128 | 1.252 |

1. A method for assessing risk of cardiovascular disease (CVD) in a subject having an inflammatory disease, the method comprising:
measuring in a sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); and
calculating a CVD risk score for the subject with an interpretation function using the protein levels, one or more clinical terms, and a set of training clinical data of a reference group, wherein the three or more biomarkers comprise LEP, TNFR1, and MMP3.

2. The method of claim 1, further comprising validating the CVD risk score with an interpretation function using the protein levels, one or more clinical terms, and a set of validation clinical data of the reference group.

3. The method of claim 1, wherein the sample is a blood sample.

4. The method of claim 1, wherein the subject is more than 40 years of age.

5. The method of claim 1, wherein the subject has no prior history of heart attack or stroke.

6. The method of claim 1, wherein the inflammatory disease is rheumatoid arthritis (RA).

7. (canceled)

8. The method of claim 1, wherein the clinical terms comprise at least one of age, sex, smoking, diabetes, hypertension, history of cardiovascular disease, gender, adiposity, body mass index, race, and ethnicity.

9. The method of claim 1, wherein the reference group is patients who have been tested for activity of rheumatoid arthritis (RA) and/or cardiovascular disease (CVD).

10. (canceled)

11. The method of claim 1, wherein the three or more biomarkers comprise LEP, TNFR1, MMP3, CRP, IL6, and SAA1.

12. The method of claim 1, wherein the three or more biomarkers comprise LEP, TNFR1, MMP3, CRP, IL6, SAA1, CHI3L1, EGF, VCAM1, MMP1, RETN, and VEGFA.

13. The method of claim 1, wherein the CVD risk score is validated with clinical data selected from a DAS score, a DAS28 score, a DAS28-CRP score, a DAS28-ESR score, a Sharp score, a tender joint count score (TJC), and a swollen joint count score (SJC).

14. The method of claim 1, wherein calculating a CVD risk score comprises calculating an Adjusted MBDA score and calculating the CVD risk score by combining the Adjusted MBDA score with the clinical terms using the interpretation function.

15. The method of claim 1, wherein the interpretation function comprises one or more of Survival Regression analysis, Cox Proportional Hazards, Box-Cox transformation, Clustering Machine Learning, Hierarchical Clustering Analysis, Centroid Clustering, Distribution Clustering, Density Clustering, Cluster Data Mining, analysis of variants (ANOVA), Ada-boosting, Classification and Regression Trees (CART), boosted CART, Random Forest (RF), Recursive Partitioning Trees (RPART), Curds and Whey (CW), Curds and Whey-Lasso, principal component analysis (PCA), factor rotation analysis, Linear Discriminant Analysis (LDA), Eigengene Linear Discriminant Analysis (ELDA), quadratic discriminant analysis, Discriminant Function Analysis (DFA), Hidden Markov Models, kernel density estimation, kernel partial least squares algorithm, kernel matching pursuit algorithm, kernel Fisher's discriminate analysis algorithm, kernel principal components analysis algorithm; linear regression, Stepwise Regression, Forward-Backward Variable Stepwise Regression, Lasso shrinkage and selection, Elastic Net regularization and selection, Lasso and Elastic Net-regularized generalized linear model, Logistic Regression (LogReg), Kth-nearest neighbor (KNN), non-linear regression, classification, neural networks, partial least square, rules based classification, shrunken centroids (SC), sliced inverse regression, Standard for the Exchange of Product model data, Application Interpreted Constructs (StepAIC), super principal component (SPC) regression, Support Vector Machines (SVM), and Recursive Support Vector Machines (RSVM), and combinations thereof.

16. The method of claim 1, wherein the interpretation function provides an algorithm which includes a hyperbolic tangent or an exponential of a biomarker score.

17. The method of claim 1, wherein the protein levels are measured by immunoassay.

18. The method of claim 1, further comprising recommending a therapy for CVD for the subject based on the CVD risk score exceeding a threshold level, or recommending no therapy for CVD based on the CVD risk score being below a threshold level.

19.-20. (canceled)

21. The method of claim 18, wherein the therapy is administering one or more medications selected from a cholesterol-reducing medication, a blood flow-increasing medication, a heart rhythm-regulating medication, a heart rhythm-stabilizing medication, a blood blockage-reducing medication, a beta-blocker, an ACE inhibitor, an aldosterone inhibitor, an angiotensin II receptor blocker, a calcium channel blocker, a cholesterol lowering drug, a diuretic, an inotropic medication, an electrolyte supplement, a PCSK9 inhibitor, and a vasodilator.

22. The method of claim 18, wherein the therapy is administering a DMARD selected from MTX, azathioprine (AZA), bucillamine (BUC), chloroquine (CQ), cyclosporine, doxycycline (DOXY), hydroxychloroquine (HCQ), intramuscular gold (IM gold), leflunomide (LEF), levofloxacin (LEV), sulfasalazine (SSZ), folinic acid, D-pencillamine, gold auranofin, gold aurothioglucose, gold thiomalate, cyclophosphamide, chlorambucil, infliximab, adalimumab, etanercept, golimumab, anakinra, abatacept, rituximab, and tocilizumab.

23.-24. (canceled)

25. The method of claim 18, wherein the threshold level is one of borderline risk threshold, intermediate risk threshold, and high risk threshold based on a CVD 3-year risk or a CVD 10-year risk.

26.-49. (canceled)

50. A kit for assessing risk of cardiovascular disease (CVD) in a subject having an inflammatory disease, the kit comprising:
reagents for measuring in a blood sample from the subject protein levels for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA); and
instructions for using the reagents for obtaining the biomarker levels.

51. A system for assessing risk of cardiovascular disease (CVD) in a subject having an inflammatory disease, the system comprising:
a processor for receiving the subject's protein levels measured in a blood sample for three or more biomarkers of a set of biomarkers comprising leptin (LEP), tumor necrosis factor receptor superfamily, member 1A (TNFR1), matrix metallopeptidase 3 (stromelysin 1, progelatinase) (MMP3), C-reactive protein, pentraxin-related (CRP), interleukin 6 (interferon, beta 2) (IL6), serum amyloid A1 (SAA1), chitinase 3-like 1 (cartilage glycoprotein-39) (CHI3L1), epidermal growth factor (beta-urogastrone) (EGF), vascular cell adhesion molecule 1 (VCAM1), matrix metallopeptidase 1 (interstitial collagenase) (MMP1), resistin (RETN), and vascular endothelial growth factor A (VEGFA);
one or more processors for carrying out the steps:
calculating a CVD risk score for the subject from the biomarker protein levels and one or more clinical terms using an interpretation function, wherein the three or more biomarkers comprise LEP, TNFR1, and MMP3;
identifying the subject having an inflammatory disease and at risk of cardiovascular disease (CVD) based on the CVD risk score exceeding a threshold level; and
a display for displaying and/or reporting the CVD risk score.

52.-54. (canceled)
